SEQUENCE LISTING (48 sequences)

SEQ ID NO: 1 - protein, 177 aa, synthetic construct
  REGION 1..177: note = Synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 98 aa, synthetic construct
  REGION 1..98: note = Synthetic construct
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 3 - DNA, 22 bp, synthetic construct
  misc_feature 1..22: note = Synthetic construct
  source 1..22: mol_type = other DNA
gtctaccaggcattcgcttcat

SEQ ID NO: 4 - DNA, 24 bp, synthetic construct
  misc_feature 1..24: note = Synthetic construct
  source 1..24: mol_type = other DNA
tcagctggaccacagccgcagcgt

SEQ ID NO: 5 - DNA, 21 bp, synthetic construct
  misc_feature 1..21: note = Synthetic construct
  source 1..21: mol_type = other DNA
tcagaaatcctttctcttgac

SEQ ID NO: 6 - DNA, 24 bp, synthetic construct
  misc_feature 1..24: note = Synthetic construct
  source 1..24: mol_type = other DNA
ctagcctctggaatcctttctctt

SEQ ID NO: 7 - protein, 8 aa, synthetic construct
  REGION 1..8: note = Synthetic construct
  VARIANT 2: note = Val or Ile
  VARIANT 4: note = Any Amino Acid
DXEXNPGP

SEQ ID NO: 8 - protein, 25 aa, synthetic construct
  REGION 1..25: note = Synthetic construct
GSGVKQTLNFDLLKLAGDVESNPGP

SEQ ID NO: 9 - DNA, 1674 bp, synthetic construct
  misc_feature 1..1674: note = Synthetic construct
  source 1..1674: mol_type = other DNA
atggagttgcctgttaggttgttggtgctgatgttctggattcctgcttccagcagtgatgtcgtgatgacccaaactccactctccctgcctgtcagtcttggagatcaagcctccatctcttgcagatctagtcagagccttgtacacagtaatggaaacacctatttacgttggtacctgcagaagccaggccagtctccaaaggtcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaaagtagtgctgatgatgctaagaaggatgctgctaagaaggatgatgctaagaaggatgatgctaagaaggatggtgaggtgaagctggatgagactggaggaggcttggtgcaacctgggaggcccatgaaactctcctgtgttgcctctggattcacttttagtgactactggatgaactgggtccgccagtctccagagaaaggactggagtgggtagcacaaattagaaacaaaccttataattatgaaacatattattcagattctgtgaaaggcagattcaccatctcaagagatgattccaaaagtagtgtctacctgcaaatgaacaacttaagagttgaagacatgggtatctattactgtacgggttcttactatggtatggactactggggtcaaggaacctcagtcaccgtctccgcggccgcagtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccctctgctgtccttgatcatcactctcatctgctaccacaggagccgaaatagtagaaggaacagactccttcaaagtgactacatgaacatgactccccggaggcctgggctcactcgaaagccttaccagccctacgcccctgccagagactttgcagcgtaccgccccaaatggatcaggaaaaaattcccccacatattcaagcaaccatttaagaagaccactggagcagctcaagaggaagatgcttgtagctgccgatgtccacaggaagaagaaggaggaggaggaggctatgagctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 10 - DNA, 146 bp, synthetic construct
  misc_feature 1..146: note = Synthetic construct
  source 1..146: mol_type = other DNA
ggaagcggagtgaaacagactttgaattttgaccttctcaagttggcgggagacgtggagtccaaccctggaccatgcataaaaagcttaaaccagttaactggaaaacgcgtaaagtcgacaaaggccaaaaaggccaacgtacg

SEQ ID NO: 11 - DNA, 864 bp, synthetic construct
  misc_feature 1..864: note = Synthetic construct
  source 1..864: mol_type = other DNA
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgacgcatgtttcctaaagagactactgagagaaataaaaacttgttggaataaaattttgaagggcagtataggaagcggagtgaaacagactttgaattttgaccttctcaagttggcgggagacgtggagtccaaccctggacctatggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctccagaccagccctgggtggatcgcatcatccgaagactgaagaagtcttctgccaagaacaaaggcaacagcaccagaaggagccctgtgtcttga

SEQ ID NO: 12 - DNA, 783 bp, synthetic construct
  misc_feature 1..783: note = Synthetic construct
  source 1..783: mol_type = other DNA
atggactggacctggcggatcctgttcctggtggctgctgctacaggcgcccacagccagatcgtgctgtctcagtctcccgccatcctgtctgctagccctggcgagaaagtgaccatgacctgcagagccagcagcagcgtgtcctacatccactggttccagcagaagcccggcagcagccccaagccttggatctacgccacaagcaacctggcctctggcgtgccagtgcggtttagcggctctggctctggcaccagctacagcctgaccatcagcagagtggaagccgaggacgccgccacctactactgtcagcagtggaccagcaacccccccacattcggcggaggcaccaagctggaaatcaagggcggaggcggatctggcggcggaggatctgggggaggcggctctcaggtgcagctgcagcagcctggcgctgagctcgtgaaacctggcgcctccgtgaagatgagctgcaaggccagcggctacaccttcacaagctacaacatgcactgggtcaagcagacccctggcagaggcctggaatggatcggcgctatctaccccggcaacggcgacacctcctacaaccagaagttcaagggcaaggccaccctgaccgccgacaagagcagcagcacagcctacatgcagctgtcctccctgaccagcgaggacagcgccgtgtactactgcgccagatctacctactacggcggcgactggtacttcaacgtgtggggcgctggcaccaccgtgaccgtgtctgct

SEQ ID NO: 13 - DNA, 1665 bp, synthetic construct
  misc_feature 1..1665: note = Synthetic construct
  source 1..1665: mol_type = other DNA
atggtccacgtctccttcagatacatcttcggcatcccccccctgatcctggtcctcctgcctgtcacctccagcgaatgtcatatcaaggacaaagagggcaaggcttatgagagcgtcctgatgatctccattgatgagctggataagatgaccggcaccgacagcaactgtcccaacaatgagcccaacttctttagaaagcacgtgtgtgacgataccaaggaggctgccttcctgaacagggccgccagaaagctgaagcagttcctgaagatgaacatttccgaggagttcaacgtgcacctcctcaccgtgagccagggcacccagacactggtcaattgcacctccaaggaggagaagaacgtgaaagagcagaaaaagaatgatgcttgtttcctcaagaggctgctgagggagatcaagacctgttggaataagatcctgaaaggcagcatcggcagcggagtcaagcaaaccctgaacttcgacctgctgaaactggccggagatgtggagagcaatcccggccctatggcccccagagtcacccctctgctggccttcagcctgctcgtgctgtggaccttccccgctcccaccctgggcggcgccaatgatgctgaggactgttgcctctccgtgacccagaggcccatccctggaaacatcgtcaaagccttcaggtacctgctcaacgaagacggatgtagggtgcctgccgtggtgttcacaacactgagaggctaccagctctgcgcccctcctgatcagccctgggtcgacagaatcatcagaaggctgaagaagtccagcgccaagaacaaaggcaatagcacaaggagaagccctgtgagcgaattcggaagcggagtgaaacagactttgaattttgaccttctcaagttggcgggagacgtggagtccaaccctggaccatgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 14 - protein, 554 aa, synthetic construct
  REGION 1..554: note = Synthetic construct
MVHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIGSGVKQTLNFDLLKLAGDVESNPGPMAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVSEFGSGVKQTLNFDLLKLAGDVESNPGPCMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - protein, 128 aa, synthetic construct
  REGION 1..128: note = VH07_aa
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGS

SEQ ID NO: 16 - protein, 116 aa, synthetic construct
  REGION 1..116: note = VL07_aa
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 17 - protein, 128 aa, synthetic construct
  REGION 1..128: note = VH36_aa
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILLS

SEQ ID NO: 18 - protein, 115 aa, synthetic construct
  REGION 1..115: note = VL36_aa
QPVLTQSSSLSASPGASASLTCTLRSGINVGPRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 19 - protein, 119 aa, synthetic construct
  REGION 1..119: note = VHMO_aa
QVQLVQSGAEVKKPGASVKVSCKASGYTFTGNYINWVRQAPGQGLEWMGIINPTKGWTLYAQKFQGRVTMTRDTSISTAYMELSSLRSEDTAVYYCARWHHGTWIFDYWGQGTLVTVSS

SEQ ID NO: 20 - protein, 109 aa, synthetic construct
  REGION 1..109: note = VLMO_aa
DIVLTQPPSVSGAPGQRVTISCSGSSSNIGSNYVSWYQQLPGTAPKLLIYNDNQRPSGVPDRFSGSKSGTSASLAITGLQSEDEADYYCSTYDRRTFSVFGGGTKLTVL

SEQ ID NO: 21 - protein, 21 aa, synthetic construct
  REGION 1..21: note = CD8 M_aa
IYIWAPLAGTCGVLLLSLVIT

SEQ ID NO: 22 - protein, 41 aa, synthetic construct
  REGION 1..41: note = CD28_A_aa
RSKRSRLLHSDYMNMTPRRPGPTRKHYQPYAPPRDFAAYRS

SEQ ID NO: 23 - protein, 46 aa, synthetic construct
  REGION 1..46: note = 4-1 BB_A_aa
RFSVVKRGRKKLLYIFKQPFMRPVQTTQEEDGCSCRFPEEEEGGCE

SEQ ID NO: 24 - protein, 113 aa, synthetic construct
  REGION 1..113: note = CD3z_A_aa
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 19 aa, synthetic construct
  REGION 1..19: note = signal peptideT_aa
MDWTWRILFLVAAATGAHS

SEQ ID NO: 26 - protein, 23 aa, synthetic construct
  REGION 1..23: note = signal peptide P_aa
MALPVTALLLPLALLLHAARPGS

SEQ ID NO: 27 - protein, 10 aa, synthetic construct
  REGION 1..10: note = VH07_CDR1_aa
GDSVSSNSAT

SEQ ID NO: 28 - protein, 9 aa, synthetic construct
  REGION 1..9: note = VH07_CDR2_aa
TYYRSKWYN

SEQ ID NO: 29 - protein, 13 aa, synthetic construct
  REGION 1..13: note = VH07_CDR3_aa
ARGMMTYYYGMDV

SEQ ID NO: 30 - protein, 9 aa, synthetic construct
  REGION 1..9: note = VL07_CDR1_aa
SGINVGPYR

SEQ ID NO: 31 - protein, 7 aa, synthetic construct
  REGION 1..7: note = VL07_CDR2_aa
YKSDSDK

SEQ ID NO: 32 - protein, 9 aa, synthetic construct
  REGION 1..9: note = VL07_CDR3_aa
MIWHSSAAV

SEQ ID NO: 33 - protein, 8 aa, synthetic construct
  REGION 1..8: note = VL36_CDR1_aa
SGINVGPR

SEQ ID NO: 34 - protein, 8 aa, synthetic construct
  REGION 1..8: note = VHMO_CDR1_aa
GYTFTGNY

SEQ ID NO: 35 - protein, 8 aa, synthetic construct
  REGION 1..8: note = VHMO_CDR2_aa
INPTKGWT

SEQ ID NO: 36 - protein, 12 aa, synthetic construct
  REGION 1..12: note = VHMO_CDR3_aa
ARWHHGTWIFDY

SEQ ID NO: 37 - protein, 8 aa, synthetic construct
  REGION 1..8: note = VLMO_CDR1_aa
SSNIGSNY

SEQ ID NO: 38 - skipped

SEQ ID NO: 39 - protein, 10 aa, synthetic construct
  REGION 1..10: note = VLMO_CDR3_aa
STYDRRTFSV

SEQ ID NO: 40 - protein, 15 aa, synthetic construct
  REGION 1..15: note = linker 15_aa
GGGGSGGGGSGGGGS

SEQ ID NO: 41 - protein, 25 aa, synthetic construct
  REGION 1..25: note = linker 25_aa
SSADDAKKDAAKKDDAKKDDAKKDG

SEQ ID NO: 42 - protein, 177 aa, Homo sapiens; identical to SEQ ID NO: 1

SEQ ID NO: 43 - protein, 98 aa, Homo sapiens; identical to SEQ ID NO: 2

SEQ ID NO: 44 - DNA, 531 bp, Homo sapiens
  source 1..531: mol_type = other DNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 45 - DNA, 294 bp, Homo sapiens
  source 1..294: mol_type = other DNA
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagt

SEQ ID NO: 46 - protein, 8 aa, synthetic construct
  REGION 1..8: note = 2A peptide cleavage region
  VARIANT 2: note = Val or Ile
  VARIANT 4: note = any amino acid
DXEXNPGP

SEQ ID NO: 47 - protein, 25 aa, synthetic construct
  REGION 1..25: note = F2A_aa
GSGVKQTLNFDLLKLAGDVESNPGP

SEQ ID NO: 48 - protein, 6 aa, synthetic construct
  REGION 1..6: note = synthetic construct
HHHHHH

(19) United States
(12) Patent Application Publication
(10) Pub. No.: US 2026/0053856 A1
(43) Pub. Date: Feb. 26, 2026

(54) IMMUNOCOMPETENT CELL AND EXPRESSION VECTOR EXPRESSING REGULATORY FACTORS OF IMMUNE FUNCTION / A CELL SURFACE MOLECULE SPECIFICALLY RECOGNIZING HUMAN MESOTHELIN, IL-7 AND CCL19

(71) Applicant: Noile-Immune Biotech, Inc., Tokyo (JP)

(72) Inventors: Koji TAMADA, Yamaguchi (JP); Yukimi SAKODA, Yamaguchi (JP); Keishi ADACHI, Yamaguchi (JP)

(73) Assignee: Noile-Immune Biotech, Inc., Tokyo (JP)

(21) Appl. No.: 19/365,120

(22) Filed: Oct. 21, 2025

Related U.S. Application Data
(63) Continuation-in-part of application No. 18/737,778, filed on Jun. 7, 2024, now abandoned.
Application No. 18/737,778 is a continuation-in-part of application No. 18/424,245, filed on Jan. 26, 2024.
Application No. 18/424,245 is a continuation of application No. 17/748,889, filed on May 19, 2022, now Pat. No. 11,931,381.
Application No. 17/748,889 is a continuation of application No. 16/084,503, now Pat. No. 11,337,997.

(30) Foreign Application Priority Data
Mar. 17, 2016 (JP) 2016-053913

(51) Int. Cl. A61K 35/17; A61K 39/00; A61K 40/11; A61K 40/32; A61K 40/42; C07K 14/54; C07K 14/725; C12N 5/0783; C12N 5/10; C12N 15/09; C12N 15/79

(52) U.S. Cl. CPC A61K 35/17; A61K 39/0011; A61K 40/11; A61K 40/32; A61K 40/42; C07K 14/5418; C07K 14/7051; C12N 5/0636; C12N 5/10; C12N 15/09; C12N 15/79

(57) ABSTRACT

An object according to certain aspect(s) of the present invention is to provide an immunocompetent cell that expresses regulatory factors of immunocompetent cell immune function and possesses all of proliferative potential, viability, and the ability to accumulate a T cell, and an expression vector of regulatory factors of immune function for generating the immunocompetent cell. An immunocompetent cell expressing a cell surface molecule specifically recognizing a cancer antigen, interleukin 7 (IL-7), and CCL19 is generated. Preferably, the cell surface molecule specifically recognizing a cancer antigen is T cell receptor specifically recognizing the cancer antigen, and the immunocompetent cell is a T cell. Another object according to certain aspect(s) of the present invention is to provide an immunocompetent cell targeting mesothelin. An immunocompetent cell that expresses a cell surface molecule specifically recognizing human mesothelin, interleukin 7 (IL-7), and chemokine (C-C motif) ligand 19 (CCL19) is produced. It is preferred that: the cell surface molecule specifically recognizing human mesothelin should be chimeric antigen receptor (CAR) having single chain antibody, a transmembrane region, and a signaling region that induces the activation of the immunocompetent cell; and the heavy chain variable region and the light chain variable region should be connected via a peptide linker consisting of a 2- to 30-amino acid sequence.

### SEQUENCE LISTING

The instant application contains a Sequence Listing which has been submitted in XML format via Patent Center and is hereby incorporated by reference in its entirety. Said XML copy is named 132281-5006-US-01_ST26.xml, created on Oct. 21, 2025 and is 67,573 bytes.

U.S. Patent Application Publication Nos. 2024/0307451 and 2024/0139248 are incorporated herein by reference in their entirety.

### TECHNICAL FIELD

The present invention relates to an immunocompetent cell that expresses a cell surface molecule specifically recognizing human mesothelin, interleukin 7 (IL-7), and chemokine (C-C motif) ligand 19 (CCL19), an anticancer agent containing the immunocompetent cell, an expression vector for generating the immunocompetent cell, a pharmaceutical composition comprising the immunocompetent cell, an expression vector comprising a nucleic acid encoding a cell surface molecule specifically recognizing mesothelin, a nucleic acid encoding IL-7, and a nucleic acid encoding CCL19, and a method for producing an immunocompetent cell that expresses a cell surface molecule specifically recognizing human mesothelin, IL-7, and CCL19, comprising introducing a nucleic acid encoding the cell surface molecule specifically recognizing human mesothelin, a nucleic acid encoding the IL-7, and a nucleic acid encoding the CCL19 to an immunocompetent cell.

### BACKGROUND ART

Malignant tumors are diseases that affect many people in the world and in general, are widely treated by chemotherapy, radiotherapy, or surgical therapy. However, there have been various problems such as the occurrence of adverse reactions, a loss of some functions, and recurrence or metastasis which cannot be treated. Accordingly, the development of immune cell therapy has been advanced in recent years in order to maintain higher quality of life (QOL) of patients. This immune cell therapy is a therapy which involves collecting immunocompetent cells from a patient, performing procedures to enhance the immune functions of the immunocompetent cells, amplifying the cells, and bringing the cells back to the patient. Specifically, a therapy of collecting T cells from a patient, introducing a nucleic acid encoding chimeric antigen receptor (hereinafter, also referred to as “CAR”) to the T cells, and bringing the T cells back to the patient (see Non-patent Document 1) is known. This therapy is currently under clinical trial worldwide and has produced results indicating efficacy on, for example, malignant hematopoietic organ tumors such as leukemia or lymphoma.

At least several hundred different types of factors such as cytokines, chemokines, and signal regulatory proteins are known as regulatory factors of immune function of immunocompetent cells such as T cells. Among them, interleukin 7 (IL-7) is a cytokine essential for the survival of T cells and is known to be produced by non-hematopoietic cells such as stromal cells of the bone marrow, the thymus, and lymphatic organs or tissues. A T cell expressing a chimeric cytokine receptor comprising IL-7 and IL-7R alpha fused with each other (see patent document 1) is disclosed as a T cell exploiting the function of this IL-7. However, the chimeric cytokine receptor in the T cell is expressed as one fusion protein in a manner limited to the membrane surface of the T cell introduced therewith, and merely transduces a signal of a cytokine such as IL-7R in a ligand-independent manner only to the autologous cell. Thus, the chimeric cytokine receptor cannot enhance the function of a T cell unintroduced with the receptor.

It is disclosed that: decreased expression of CCL19, CCL21, and IL-7 is responsible for deficiency in the maintenance of a T cell zone in the spleen of an SIRP alpha mutant mouse (see non-patent document 2); and CCL19, CCL21, and IL-7 work to maintain the homeostasis of T cells in secondary lymphoid tissues (spleen tissues or lymph nodes) (see non-patent document 3). However, non-patent documents 2 and 3 described above show an effect on nonactivated T cells constantly present in the T cell zones of secondary lymphoid tissues and do not show the direct relationship with antitumor immune response. Furthermore, CCL19-, CCL21-, or IL-7-expressing cells described in non-patent documents 2 and 3 were not T cells but were reticuloendothelial cells present in the secondary lymphoid tissues.

Meanwhile, T cell receptor (hereinafter, also referred to as “TCR”) is an antigen receptor molecule expressed on the cell membranes of T cells. TCR is present as a heterodimer consisting of an alpha chain and a beta chain, or of a gamma chain and a delta chain and is known to activate T cells by recognizing an antigen molecule bound with a major histocompatibility complex (MHC) molecule.

Immunotherapy which involves introducing a gene of TCR capable of recognizing a tumor antigen expressed on cancer cells to T cells obtained from cancer patients, amplifying the T cells, and then transferring the T cells again to the patients is under development by the application of the function of this TCR. Specifically, a pharmaceutical composition for meningioma treatment containing a cell expressing TCR specifically recognizing a WT1-expressing cell (see patent document 2) is disclosed.

Although some of the techniques described above exhibit an antitumor effect on malignant tumor in the hematopoietic organ, none of the previous cases still exhibit a marked effect on solid cancer. This is considered to be due to the problems of low survival efficiency of transferred immunocompetent cells in vivo or insufficient activation of endogenous immunocompetent cells induced by transferred immunocompetent cells or insufficient local accumulation thereof to tumor. Thus, the development of a technique of solving these problems has been desired.

Meanwhile, the present inventors have proposed immune cell therapy of markedly suppressing solid cancer by co-expressing IL-7 and CCL19 (see Patent Documents 3 and 4). This method can enhance the activation of endogenous immunocompetent cells or their ability to accumulate on tumor cells.

Mesothelin is known to be expressed in cells of cancer such as mesothelioma, colorectal cancer (rectum cancer and colon cancer), pancreatic cancer, ovary cancer, lung cancer, breast cancer, or head and neck cancer. CAR-T cells targeting the mesothelin are disclosed (see Patent Documents 5 and 6).

### PRIOR ART DOCUMENTS

### Patent Documents

Patent Document 1: International Publication No. WO 2013/123061
Patent Document 2: Japanese unexamined Patent Application Publication No. 2013-116891
Patent Document 3: International Publication No. WO 2016/056228
Patent Document 4: International Publication No. WO 2017/159736
Patent Document 5: U.S. Patent Application Publication No. 2014/0301993
Patent Document 6: Japanese unexamined Patent Application Publication No. 2017-518053

### Non-Patent Documents

Non-patent Document 1: Yozo Nakazawa, The Shinshu Medical Journal 61 (4): 197-203 (2013)
Non-patent Document 2: SATO-HASHIMOTO M. et al., J. Immunol., 2011, vol. 187, no. 1, 291-7
Non-patent Document 3: SIEGERT S. et al., Front. Immunol., 2012, vol. 3, article 285

### SUMMARY OF THE INVENTION

### Object to be Solved by the Invention

Immunocompetent cells for use in conventional immunotherapy do not sufficiently potentiate the immunity-inducing effect of endogenous immunocompetent cells or the proliferative potential, survival capacity, or the ability of immunocompetent cells to accumulate a T cell. Accordingly, an object according to certain aspect(s) of the present invention is to provide an immunocompetent cell that expresses regulatory factors of immunocompetent cell immune function and possesses all of proliferative potential, survival capacity, and the ability to accumulate a T cell, and an expression vector of regulatory factors of immune function for generating the immunocompetent cell. Further. the development of techniques that can be adapted to solid cancer found to receive no sufficient therapeutic effects of conventional immune cell therapy are underway by developing immunocompetent cell therapy using CAR-expressing T cells, TCR-expressing T cells, or the like that co-express IL-7 and CCL19, as described above, and markedly improving the ability of immunocompetent cells to proliferate, the ability of immunocompetent cells to survive, or the ability of host's immunocompetent cells to accumulate. On the other hand, the development of CAR-expressing immunocompetent cells targeting mesothelin highly expressed in cancer (e.g., mesothelioma and pancreatic cancer) cells has not yet produced satisfactory clinical results due to insufficient local accumulation of immunocompetent cells on cancer, and a risk of recurrence of tumor ascribable to short exertion of antitumor effects, etc. Accordingly, an object according to certain aspect(s) of the present invention is to provide a novel immunocompetent cell targeting mesothelin.

### Means to Solve the Object

The inventors have attempted to improve cells expressing regulatory factors of immune function for the purpose of achieving a much better immunity-inducing effect or antitumor activity in cancer immunotherapy using immunocompetent cells. During the course thereof, the inventors have focused on cytokines, chemokines, and signal regulatory proteins which are factors regulating the immune function of immunocompetent cells, and constructed a vector for the expression of the factors regulating the immune function of immunocompetent cells. As a result of introducing this expression vector to immunocompetent cells, the inventors have found that immunocompetent cells superior in immunity-inducing effect, proliferative potential, survival capacity and the ability to accumulate a T cell to the conventional immunocompetent cells can be generated, and thereby completed the present invention.

Specifically, aspects of the present invention are as disclosed in the following items (1) to (9):

(1) An immunocompetent cell expressing a cell surface molecule specifically recognizing a cancer antigen, interleukin 7 (IL-7), and CCL19.
(2) The immunocompetent cell according to (1), wherein the cell surface molecule specifically recognizing a cancer antigen is T cell receptor specifically recognizing the cancer antigen.
(3) The immunocompetent cell according to (1) or (2), wherein the immunocompetent cell is a T cell.
(4) The immunocompetent cell according to any one of (1) to
(3), wherein the cancer antigen is WT1, MART-1, NY-ESO-1, MAGE-A1, MAGE-A3, MAGE-A4, Glypican-3, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GD2, or GM2.
(5) An expression vector for generating an immunocompetent cell according to any one of (1) to (4), the expression vector being any of the following expression vectors (a) to (e):
(a) an expression vector containing a nucleic acid encoding a cell surface molecule specifically recognizing a cancer antigen, a nucleic acid encoding IL-7, and a nucleic acid encoding CCL19;
(b) the following two expression vectors (b-1) and (b-2):
   (b-1) an expression vector containing a nucleic acid encoding a cell surface molecule specifically recognizing a cancer antigen; and
   (b-2) an expression vector containing a nucleic acid encoding IL-7 and a nucleic acid encoding CCL19;
(c) the following two expression vectors (c-1) and (c-2):
   (c-1) an expression vector containing a nucleic acid encoding a cell surface molecule specifically recognizing a cancer antigen, and a nucleic acid encoding IL-7; and
   (c-2) an expression vector containing a nucleic acid encoding CCL19;
(d) the following two expression vectors (d-1) and (d-2):
   (d-1) an expression vector containing a nucleic acid encoding IL-7; and
   (d-2) an expression vector containing a nucleic acid encoding a cell surface molecule specifically recognizing a cancer antigen, and a nucleic acid encoding CCL19; and
(e) the following three expression vectors (e-1), (e-2) and (e-3):
   (e-1) an expression vector containing a nucleic acid encoding a cell surface molecule specifically recognizing a cancer antigen;
   (e-2) an expression vector containing a nucleic acid encoding IL-7; and
   (e-3) an expression vector containing a nucleic acid encoding CCL19.
      (6) The expression vector according to (5), wherein the cell surface molecule specifically recognizing a cancer antigen is T cell receptor specifically recognizing the cancer antigen.
      (7) The expression vector according to (5) or (6), wherein the nucleic acid encoding a cell surface molecule specifically recognizing a cancer antigen, the nucleic acid encoding IL-7, and the nucleic acid encoding CCL19 in the expression vector (a)
the nucleic acid encoding IL-7 and the nucleic acid encoding CCL19 in the expression vector (b-2)
the nucleic acid encoding a cell surface molecule specifically recognizing a cancer antigen, and the nucleic acid encoding IL-7 in the expression vector (c-1), or
the nucleic acid encoding a cell surface molecule specifically recognizing a cancer antigen, and the nucleic acid encoding CCL19 in the expression vector (d-2)
are linked via a sequence encoding a self-cleaving peptide.
   (8) The expression vector according to any one of (5) to (7), wherein the expression vector contains a nucleic acid encoding a suicide gene.
   (9) An anticancer agent comprising an immunocompetent cell according to any one of (1) to (4) and a pharmaceutically acceptable additive.

The present inventors also have studied the further possibility of our own previously developed T cells that express CAR, IL-7 and CCL19. As a result, the present inventors have completed additional aspects of the present invention by finding that CAR containing single chain antibody specifically binding to human mesothelin and containing a particular amino acid sequence specifically recognizing human mesothelin as a cell surface molecule can be selectively used to exert cytotoxic activity against cancer cells expressing mesothelin and to suppress reduction in survival rate caused by tumor formed by the cancer cells expressing mesothelin.

Specifically, the additional aspects of the present invention is as follows:

[1] An immunocompetent cell that expresses a cell surface molecule specifically recognizing human mesothelin, interleukin 7 (IL-7), and chemokine (C-C motif) ligand 19 (CCL19).
[2] The immunocompetent cell according to [1], wherein the immunocompetent cell is an immunocompetent cell separated from a living body.
[3] The immunocompetent cell according to [1] or [2], wherein the immunocompetent cell comprises an exogenous nucleic acid encoding a cell surface molecule specifically recognizing human mesothelin, an exogenous nucleic acid encoding IL-7, and an exogenous nucleic acid encoding CCL19.
[4] The immunocompetent cell according to [3], wherein the exogenous nucleic acid encoding IL-7, and the exogenous nucleic acid encoding CCL19 are an exogenous nucleic acid encoding human IL-7, and an exogenous nucleic acid encoding human CCL19.
[5] The immunocompetent cell according to [3] or [4], wherein the exogenous nucleic acid encoding a cell surface molecule specifically recognizing human mesothelin, the exogenous nucleic acid encoding IL-7, and the exogenous nucleic acid encoding CCL19 are integrated in a genome.
[6] The immunocompetent cell according to any one of [1] to [5], wherein the cell surface molecule specifically recognizing human mesothelin is a chimeric antigen receptor (CAR) having a single chain antibody, a transmembrane region, and a signaling region that induces activation of the immunocompetent cell.
[7] The immunocompetent cell according to [6], wherein the single chain antibody in the CAR is any of the following single chain antibodies:
(1-1) a single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 30, light chain CDR2 consisting of the amino acid sequence shown by SEQ ID NO: 31, and light chain CDR3 consisting of the amino acid sequence shown by SEQ ID NO: 32;
(2-1) a single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 33, light chain CDR2 consisting of the amino acid sequence shown by SEQ ID NO: 31, and light chain CDR3 consisting of the amino acid sequence shown by SEQ ID NO: 32; and
(3-1) a single chain antibody comprising a heavy chain variable region comprising heavy chain CDR1 consisting of the amino acid sequence shown by SEQ ID NO: 34, heavy chain CDR2 consisting of the amino acid sequence shown by SEQ ID NO: 35, and heavy chain CDR3 consisting of the amino acid sequence shown by SEQ ID NO: 36, and a light chain variable region comprising light chain CDR1 consisting of the amino acid sequence shown by SEQ ID NO: 37, light chain CDR2 consisting of the amino acid sequence shown by SEQ ID NO: 38, and light chain CDR3 consisting of the amino acid sequence shown by SEQ ID NO: 39.
   [8] The immunocompetent cell according to [6] or [7], wherein the single chain antibody in the CAR is any of the following single chain antibodies:
(1-2) a single chain antibody comprising a heavy chain variable region consisting of an amino acid sequence having 85% or higher sequence identity to the amino acid sequence shown by SEQ ID NO: 15, and a light chain variable region consisting of an amino acid sequence having 85% or higher sequence identity to the amino acid sequence shown by SEQ ID NO: 2;
(2-2) a single chain antibody comprising a heavy chain variable region consisting of an amino acid sequence having 85% or higher sequence identity to the amino acid sequence shown by SEQ ID NO: 17, and a light chain variable region consisting of an amino acid sequence having 85% or higher sequence identity to the amino acid sequence shown by SEQ ID NO: 18;
(3-2) a single chain antibody comprising a heavy chain variable region consisting of an amino acid sequence having 85% or higher sequence identity to the amino acid sequence shown by SEQ ID NO: 19, and a light chain variable region consisting of an amino acid sequence having 85% or higher sequence identity to the amino acid sequence shown by SEQ ID NO: 20;
(4-2) a single chain antibody comprising a heavy chain variable region consisting of an amino acid sequence having 85% or higher sequence identity to the amino acid sequence shown by SEQ ID NO: 15, and a light chain variable region consisting of an amino acid sequence having 85% or higher sequence identity to the amino acid sequence shown by SEQ ID NO: 18; and
(5-2) a single chain antibody comprising a heavy chain variable region consisting of an amino acid sequence having 85% or higher sequence identity to the amino acid sequence shown by SEQ ID NO: 17, and a light chain variable region consisting of an amino acid sequence having 85% or higher sequence identity to the amino acid sequence shown by SEQ ID NO: 2.
   [9] The immunocompetent cell according to any one of [6] to [8], wherein the single chain antibody in the CAR is any of the following single chain antibodies:
(1-3) a single chain antibody comprising a heavy chain variable region consisting of the amino acid sequence shown by SEQ ID NO: 15, and a light chain variable region consisting of the amino acid sequence shown by SEQ ID NO: 2;
(2-3) a single chain antibody comprising a heavy chain variable region consisting of the amino acid sequence shown by SEQ ID NO: 17, and a light chain variable region consisting of the amino acid sequence shown by SEQ ID NO: 18;
(3-3) a single chain antibody comprising a heavy chain variable region consisting of the amino acid sequence shown by SEQ ID NO: 19, and a light chain variable region consisting of the amino acid sequence shown by SEQ ID NO: 20;
(4-3) a single chain antibody comprising a heavy chain variable region consisting of the amino acid sequence shown by SEQ ID NO: 15, and a light chain variable region consisting of the amino acid sequence shown by SEQ ID NO: 18; and
(5-3) a single chain antibody comprising a heavy chain variable region consisting of the amino acid sequence shown by SEQ ID NO: 17, and a light chain variable region consisting of the amino acid sequence shown by SEQ ID NO: 2.
   [10] The immunocompetent cell according to any one of [6] to [9], wherein the transmembrane region in the CAR comprises an amino acid sequence having 85% or higher sequence identity to the amino acid sequence shown by SEQ ID NO: 21.
   [11] The immunocompetent cell according to any one of [6] to [10], wherein the signaling region that induces the activation of the immunocompetent cell in the CAR comprises the amino acid sequences shown by SEQ ID NOs: 8, 9 and 10.
   [12] The immunocompetent cell according to any one of [7] to [11], wherein the heavy chain variable region and the light chain variable region are connected via a peptide linker consisting of a 2- to 30-amino acid sequence.
   [13] The immunocompetent cell according to [12], wherein the peptide linker consists of the amino acid sequence shown by SEQ ID NO: 40 or SEQ ID NO: 41.
   [14] The immunocompetent cell according to any one of [6] to [13], wherein the single chain antibody in the CAR is any of the following single chain antibodies:
(1-4) a single chain antibody sequentially comprising a heavy chain variable region consisting of the amino acid sequence shown by SEQ ID NO: 15, a peptide linker consisting of the amino acid sequence shown by SEQ ID NO: 40, and a light chain variable region consisting of the amino acid sequence shown by SEQ ID NO: 2;
(2-4) a single chain antibody sequentially comprising a heavy chain variable region consisting of the amino acid sequence shown by SEQ ID NO: 17, a peptide linker consisting of the amino acid sequence shown by SEQ ID NO: 40, and a light chain variable region consisting of the amino acid sequence shown by SEQ ID NO: 18;
(3-4) a single chain antibody sequentially comprising a light chain variable region consisting of the amino acid sequence shown by SEQ ID NO: 2, a peptide linker consisting of the amino acid sequence shown by SEQ ID NO: 40, and a heavy chain variable region consisting of the amino acid sequence shown by SEQ ID NO: 15;
(4-4) a single chain antibody sequentially comprising a heavy chain variable region consisting of the amino acid sequence shown by SEQ ID NO: 15, a peptide linker consisting of the amino acid sequence shown by SEQ ID NO: 41, and a light chain variable region consisting of the amino acid sequence shown by SEQ ID NO: 2;
(5-4) a single chain antibody sequentially comprising a light chain variable region consisting of the amino acid sequence shown by SEQ ID NO: 2, a peptide linker consisting of the amino acid sequence shown by SEQ ID NO: 41, and a heavy chain variable region consisting of the amino acid sequence shown by SEQ ID NO: 15;
(6-4) a single chain antibody sequentially comprising a heavy chain variable region consisting of the amino acid sequence shown by SEQ ID NO: 19, a peptide linker consisting of the amino acid sequence shown by SEQ ID NO: 40, and a light chain variable region consisting of the amino acid sequence shown by SEQ ID NO: 20;
(7-4) a single chain antibody sequentially comprising a light chain variable region consisting of the amino acid sequence shown by SEQ ID NO: 20, a peptide linker consisting of the amino acid sequence shown by SEQ ID NO: 40, and a heavy chain variable region consisting of the amino acid sequence shown by SEQ ID NO: 19;
(8-4) a single chain antibody sequentially comprising a heavy chain variable region consisting of the amino acid sequence shown by SEQ ID NO: 19, a peptide linker consisting of the amino acid sequence shown by SEQ ID NO: 41, and a light chain variable region consisting of the amino acid sequence shown by SEQ ID NO: 20; and
(9-4) a single chain antibody sequentially comprising a light chain variable region consisting of the amino acid sequence shown by SEQ ID NO: 20, a peptide linker consisting of the amino acid sequence shown by SEQ ID NO: 41, and a heavy chain variable region consisting of the amino acid sequence shown by SEQ ID NO: 19.
   [15] The immunocompetent cell according to any one of [6] to [14], wherein the signaling region that induces the activation of the immunocompetent cell in the CAR comprises a polypeptide of a CD28 intracellular region, a polypeptide of a 4-1BB intracellular region, and a polypeptide of a CD3ζ intracellular region.
   [16] The immunocompetent cell according to any one of [1] to [15], wherein the immunocompetent cell is a T cell.
   [17] The immunocompetent cell according to any one of [1] to [16], wherein the immunocompetent cell is derived from a human or is a T cell separated from a human.
   [18] A pharmaceutical composition comprising an immunocompetent cell according to any one of [1] to [17] and a pharmaceutically acceptable additive.
   [19] The pharmaceutical composition according to [18] for use in the treatment of cancer.
   [20] An expression vector comprising a nucleic acid encoding a cell surface molecule specifically recognizing human mesothelin, a nucleic acid encoding IL-7, and a nucleic acid encoding CCL19.
   [21] A method for producing an immunocompetent cell that expresses a cell surface molecule specifically recognizing human mesothelin, IL-7, and CCL19, comprising introducing a nucleic acid encoding the cell surface molecule specifically recognizing human mesothelin, a nucleic acid encoding the IL-7, and a nucleic acid encoding the CCL19 to an immunocompetent cell.

### Effect of the Invention

The immunocompetent cell expressing a cell surface molecule specifically recognizing a cancer antigen, IL-7, and CCL19 (hereinafter, also referred to as the “IL-7×CCL19-expressing immunocompetent cell”) according to certain aspects of the present invention has antitumor activity, and use of this immunocompetent cell enables the suppression of decrease in survival rate caused by tumor formed by a cancer cell having the antigen specifically recognized by the cell surface molecule. Also, use of the expression vector of the present invention enables the generation of an immunocompetent cell that possesses all of proliferative potential, survival capacity and the ability to accumulate a T cell. Further, the immunocompetent cell of additional aspects of the present invention has cytotoxic activity against cancer cells expressing human mesothelin and is capable of suppressing the formation of tumor expressing human mesothelin. Also, the immunocompetent cell according to certain aspect(s) of the present invention has suppressive effects on the recurrence of cancer cells.

### BRIEF DESCRIPTION OF DRAWINGS

FIG. 1 is a diagram showing the map of an IL-7×CCL19 expression vector.

FIG. 2A is a diagram showing results of examining the cell number of an IL-7/CCL19-expressing T cell.

FIG. 2B is a diagram showing results of examining the survival rate of the IL-7/CCL19-expressing T cell.

FIG. 3 is a diagram showing results of a T cell migration test using the IL-7/CCL19-expressing T cell.

FIG. 4 is a diagram showing the map of an IL-7×CCL19×HSV-TK expression vector.

FIG. 5 is a diagram showing the map of a TCR×IL-7×CCL19 expression vector.

FIG. 6 is a diagram showing the map of an IL-7×CCL19×eGFP expression vector.

FIG. 7 is a diagram showing the survival rates of an untreated mouse, a mouse given a P1A-specific TCR/eGFP-expressing T cell, and a mouse given a P1A-specific TCR/IL-7/CCL19/eGFP-expressing T cell.

FIG. 8 is a diagram showing results of examining the tumor volumes of an untreated mouse, a mouse given a P1A-specific TCR/eGFP-expressing T cell, and a mouse given a P1A-specific TCR/IL-7/CCL19/eGFP-expressing T cell.

FIGS. 9A-9B are diagrams showing the arrangements (FIG. 9A) and amino acid sequences (FIG. 9B) of 9 types of anti-human mesothelin scFvs produced in Example 7.

FIGS. 10A-10D are diagrams showing results of examining the expression of CAR in anti-human mesothelin CAR-IL-7/CCL19-expressing T cells in Example 8. FIG. 10A shows results about CAR, IL-7, and CCL19 non-expressing T cells (Non-infection), and FIGS. 10B to 10D show results about anti-human mesothelin CAR-IL-7/CCL19-expressing T cells having VH07(15)VL07 (signal peptide T), VH07(15)VL07 (signal peptide P), and VH36(15)VL36, respectively, as a scFv region.

FIGS. 11A-11E are diagrams showing results of examining the expression of CAR in anti-human mesothelin CAR-IL-7/CCL19-expressing T cells in Example 8. FIG. 11A shows results about CAR, IL-7, and CCL19 non-expressing T cells (Non-infection), and FIGS. 11B to 11E show results about anti-human mesothelin CAR-I9L-7/CCL19-expressing T cells having VH07(15)VL07, VL07(15)VH07, VH07(25)VL07, and VL07(25)VH07, respectively, as a scFv region.

FIG. 12 is a diagram confirming the expression level of mesothelin in tumor cell lines using a flow cytometer in Example 9.

FIG. 13 is an illustrative diagram of a co-culture test on anti-human mesothelin CAR-IL-7/CCL19-expressing T cells and a mesothelin-positive tumor cell line or a mesothelin-negative tumor cell line in Example 10.

FIG. 14A is a diagram showing results of measuring a surviving tumor cell line ACC-MESO-1 by flow cytometry in Example 10.

FIG. 14B is a diagram showing results of measuring a surviving tumor cell line NCI-H2052 by flow cytometry in Example 10.

FIG. 14C is a diagram showing results of measuring a surviving tumor cell line A498 by flow cytometry in Example 10.

FIG. 15A is a diagram showing results of measuring produced IFN-γ after co-culture of anti-human mesothelin CAR-IL-7/CCL19-expressing T cells and a mesothelin-positive tumor cell line ACC-MESO-1 in Example 10.

FIG. 15B is a diagram showing results of measuring produced IFN-γ after co-culture of anti-human mesothelin CAR-IL-7/CCL19-expressing T cells and a mesothelin-positive tumor cell line NCI-H2052 in Example 10.

FIG. 15C is a diagram showing results of measuring produced IFN-γ after co-culture of anti-human mesothelin CAR-IL-7/CCL19-expressing T cells and a mesothelin-negative tumor cell line A498 in Example 10.

FIG. 16 is a diagram showing results of measuring surviving leukocytes (Lymphocyte number) or PAN02 tumor cell line by flow cytometry in Example 11.

FIG. 17 is a diagram showing results of measuring produced IFN-γ after co-culture of anti-mesothelin CAR-IL-7/CCL19-expressing T cells and a PAN02 tumor cell line in Example 11.

FIG. 18 is a diagram showing results of measuring survival rates by the administration of anti-mesothelin CAR-mouse IL-7/mouse CCL19-expressing mouse T cells to tumor model mice in Example 12.

FIG. 19 is a diagram showing results of measuring tumor volumes by the administration of anti-mesothelin CAR-mouse IL-7/mouse CCL19-expressing mouse T cells to tumor model mice in Example 12.

FIG. 20A is a diagram showing results of photographing mice on days 1, 3, 7, 10, 14, 21, 31, and 38 for an exposure time of 30 seconds in Example 13 when the day on which ACC-MESO-1-GFP-Luc was administered was defined as day 1.

FIG. 20B is a diagram showing results of photographing mice on days 45, 59, 73, 87, 101, 115, 129, and 143 for an exposure time of 30 seconds in Example 13 when the day on which ACC-MESO-1-GFP-Luc was administered was defined as day 1. The photograph on day 129 of the second individual counted from the left is omitted because the individual died.

FIG. 21 is a graph showing the relationship between the number of days from administration and a mouse survival rate in Example 13.

FIG. 22 is a graph showing the relationship between the number of days from administration and the total quantity of fluorescence in Example 13.

### MODE OF CARRYING OUT THE INVENTION

The inventors have attempted to improve cells expressing regulatory factors of immune function for the purpose of achieving a much better immunity-inducing effect or antitumor activity in cancer immunotherapy using immunocompetent cells. During the course thereof, the inventors have focused on cytokines, chemokines, and signal regulatory proteins which are factors regulating the immune function of immunocompetent cells, and constructed a vector for the expression of the factors regulating the immune function of immunocompetent cells. As a result of introducing this expression vector to immunocompetent cells, the inventors have found that immunocompetent cells superior in immunity-inducing effect, proliferative potential, survival capacity and the ability to accumulate a T cell to the conventional immunocompetent cells can be generated, and thereby completed the present invention.

Specifically, the present invention is as disclosed in the following items (1) to (9):

(1) An immunocompetent cell expressing a cell surface molecule specifically recognizing a cancer antigen, interleukin 7 (IL-7), and CCL19.
(2) The immunocompetent cell according to (1), wherein the cell surface molecule specifically recognizing a cancer antigen is T cell receptor specifically recognizing the cancer antigen.
(3) The immunocompetent cell according to (1) or (2), wherein the immunocompetent cell is a T cell.
(4) The immunocompetent cell according to any one of (1) to (3), wherein the cancer antigen is WT1, MART-1, NY-ESO-1, MAGE-A1, MAGE-A3, MAGE-A4, Glypican-3, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GD2, or GM2.
(5) An expression vector for generating an immunocompetent cell according to any one of (1) to (4), the expression vector being any of the following expression vectors (a) to (e):
(a) an expression vector containing a nucleic acid encoding a cell surface molecule specifically recognizing a cancer antigen, a nucleic acid encoding IL-7, and a nucleic acid encoding CCL19;
(b) the following two expression vectors (b-1) and (b-2):
   (b-1) an expression vector containing a nucleic acid encoding a cell surface molecule specifically recognizing a cancer antigen; and
   (b-2) an expression vector containing a nucleic acid encoding IL-7 and a nucleic acid encoding CCL19;
(c) the following two expression vectors (c-1) and (c-2):
   (c-1) an expression vector containing a nucleic acid encoding a cell surface molecule specifically recognizing a cancer antigen, and a nucleic acid encoding IL-7; and
   (c-2) an expression vector containing a nucleic acid encoding CCL19;
(d) the following two expression vectors (d-1) and (d-2):
   (d-1) an expression vector containing a nucleic acid encoding IL-7; and
   (d-2) an expression vector containing a nucleic acid encoding a cell surface molecule specifically recognizing a cancer antigen, and a nucleic acid encoding CCL19; and
(e) the following three expression vectors (e-1), (e-2) and (e-3):
   (e-1) an expression vector containing a nucleic acid encoding a cell surface molecule specifically recognizing a cancer antigen;
   (e-2) an expression vector containing a nucleic acid encoding IL-7; and
   (e-3) an expression vector containing a nucleic acid encoding CCL19.
      (6) The expression vector according to (5), wherein the cell surface molecule specifically recognizing a cancer antigen is T cell receptor specifically recognizing the cancer antigen.
      (7) The expression vector according to (5) or (6), wherein the nucleic acid encoding a cell surface molecule specifically recognizing a cancer antigen, the nucleic acid encoding IL-7, and the nucleic acid encoding CCL19 in the expression vector (a)
the nucleic acid encoding IL-7 and the nucleic acid encoding CCL19 in the expression vector (b-2)
the nucleic acid encoding a cell surface molecule specifically recognizing a cancer antigen, and the nucleic acid encoding IL-7 in the expression vector (c-1), or
the nucleic acid encoding a cell surface molecule specifically recognizing a cancer antigen, and the nucleic acid encoding CCL19 in the expression vector (d-2)
are linked via a sequence encoding a self-cleaving peptide.
   (8) The expression vector according to any one of (5) to (7), wherein the expression vector contains a nucleic acid encoding a suicide gene.
   (9) An anticancer agent comprising an immunocompetent cell according to any one of (1) to (4) and a pharmaceutically acceptable additive.

### Effect of the Invention

The immunocompetent cell expressing a cell surface molecule specifically recognizing a cancer antigen, IL-7, and CCL19 (hereinafter, also referred to as the “IL-7×CCL19-expressing immunocompetent cell”) according to the present invention has antitumor activity, and use of this immunocompetent cell enables the suppression of decrease in survival rate caused by tumor formed by a cancer cell having the antigen specifically recognized by the cell surface molecule. Also, use of the expression vector of the present invention enables the generation of an immunocompetent cell that possesses all of proliferative potential, survival capacity and the ability to accumulate a T cell.

### BRIEF DESCRIPTION OF DRAWINGS

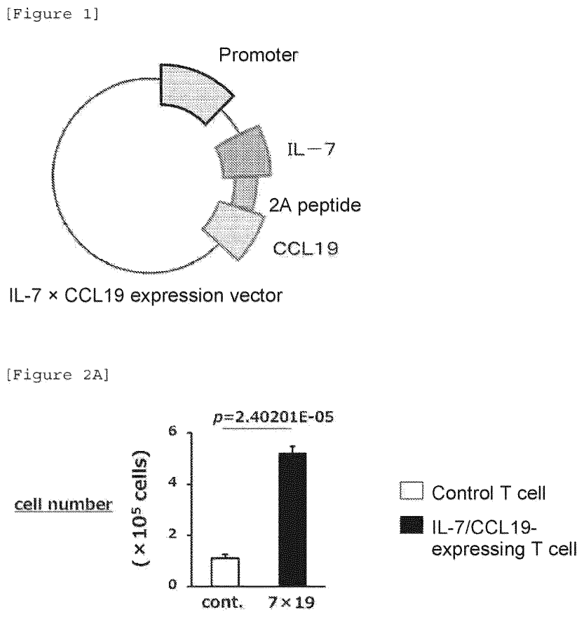

FIG. 1 is a diagram showing the map of an IL-7×CCL19 expression vector.

FIG. 2A is a diagram showing results of examining the cell number of an IL-7/CCL19-expressing T cell.

FIG. 2B is a diagram showing results of examining the survival rate of the IL-7/CCL19-expressing T cell.

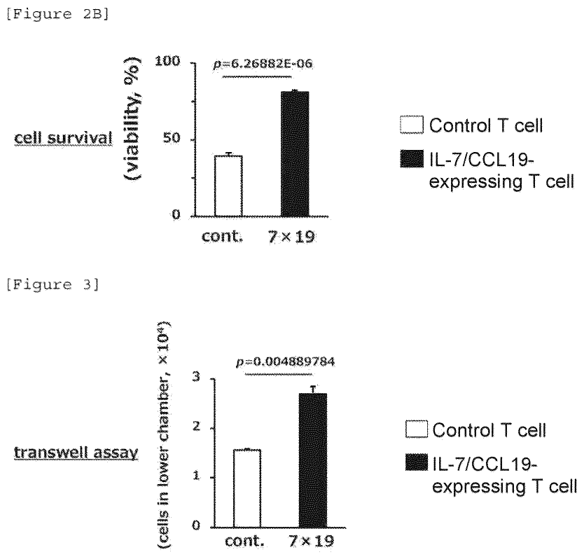

FIG. 3 is a diagram showing results of a T cell migration test using the IL-7/CCL19-expressing T cell.

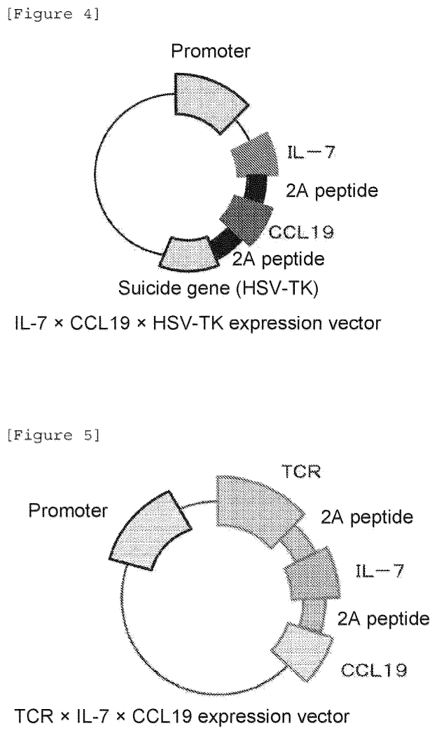

FIG. 4 is a diagram showing the map of an IL-7×CCL19×HSV-TK expression vector.

FIG. 5 is a diagram showing the map of a TCR×IL-7×CCL19 expression vector.

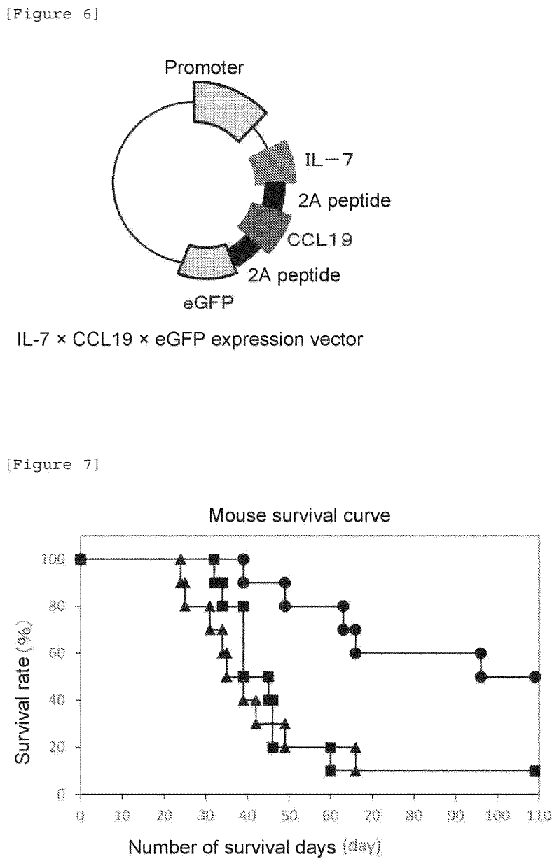

FIG. 6 is a diagram showing the map of an IL-7×CCL19×eGFP expression vector.

FIG. 7 is a diagram showing the survival rates of an untreated mouse, a mouse given a P1A-specific TCR/eGFP-expressing T cell, and a mouse given a P1A-specific TCR/IL-7/CCL19/eGFP-expressing T cell.

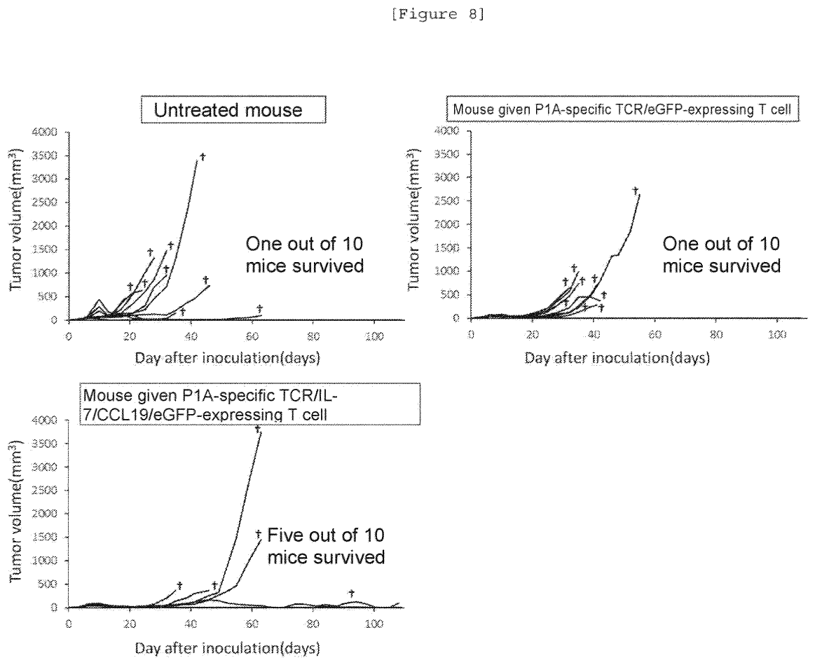

FIG. 8 is a diagram showing results of examining the tumor volumes of an untreated mouse, a mouse given a P1A-specific TCR/eGFP-expressing T cell, and a mouse given a P1A-specific TCR/IL-7/CCL19/eGFP-expressing T cell.

### MODE OF CARRYING OUT THE INVENTION

The IL-7×CCL19-expressing immunocompetent cell according to certain aspect(s) of the present invention is not particularly limited as long as the immunocompetent cell expresses a cell surface molecule specifically recognizing a cancer antigen, interleukin 7 (IL-7), and CCL19. The IL-7×CCL19-expressing immunocompetent cell according to certain aspect(s) of the present invention may further express other regulatory factors of immune function, such as IL-15, CCL21, IL-2, IL-4, IL-12, IL-13, IL-17, IL-18, IP-10, CCL4, Flt3L, interferon-gamma, MIP-1 alpha, GM-CSF, M-CSF, TGF-beta, and TNF-alpha.

The cancer antigen means a substance, such as a protein or a glycolipid, which is more highly expressed in a cancer cell than in a normal cell or specifically expressed in a cancer cell. Examples of such a cancer antigen can include a tumor-associated antigen, a cancer testis antigen, an angiogenesis-associated antigen, and an epitope peptide of a cancer neoantigen ascribable to gene mutation and can specifically include, but are not limited to, a protein such as WT1, MART-1, NY-ESO-1, MAGE-A1, MAGE-A3, MAGE-A4, Glypican-3, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, and P1A, and a glycolipid such as GD2 and GM2.

Examples of the cell surface molecule specifically recognizing a cancer antigen can include a cell surface receptor, an artificial receptor, and an adhesion factor specifically recognizing the cancer antigen and can preferably include a molecule that confers the ability to specifically mark cancer through its expression on cell surface, such as T cell receptor specifically recognizing the cancer antigen and chimeric antigen receptor (CAR) specifically recognizing the cancer antigen, more preferably TCR. TCR may be a heterodimer consisting of an alpha chain and a beta chain (alpha-beta TCR) or may be a heterodimer consisting of a gamma chain and a delta chain (gamma-delta TCR) as long as TCR specifically recognizes the cancer antigen. The cell surface molecule specifically recognizing a cancer antigen may indirectly recognize the cancer antigen as long as the recognition is specific. For example, a molecule (e.g., an antibody) specifically recognizing the cancer antigen is administered to a subject concurrently or continuously with the immunocompetent cell according to certain aspect(s) of the present invention, and the immunocompetent cell according to certain aspect(s) of the present invention is capable of indirectly specifically recognizing the cancer antigen, by recognizing the molecule (e.g., an antibody) or recognizing a tag labeling the molecule (e.g., an antibody). In the case of recognizing the antibody, examples of the cell surface molecule include CD16. Examples of the tag labeling the molecule (e.g., an antibody) include FITC.

The type of the immunocompetent cell for the IL-7×CCL19-expressing immunocompetent cell according to certain aspect(s) of the present invention can be any cell involved in immune response. Examples thereof can include: a lymphoid cell such as a T cell, a natural killer cell (NK cell), and a B cell; an antigen-presenting cell such as a monocyte, a macrophage, and a dendritic cell; and a granulocyte such as a neutrophil, an eosinophil, a basophil, and a mast cell and can preferably include a mammal (e.g., human, dog, cat, pig, or mouse)-derived T cell, more preferably a human-derived T cell. Alternatively, the T cell can be obtained by isolation and purification from a body fluid such as blood or bone marrow fluid, a tissue of the spleen, the thymus, lymph nodes, or the like, or an immunocyte infiltrating a cancer tissue of primary tumor, metastatic tumor, cancerous ascites, or the like. A T cell generated from an ES cell or an iPS cell may be used. Examples of such a T cell can include an alpha-beta T cell, a gamma-delta T cell, a CD8⁺ T cell, a CD4⁺ T cell, a tumor-infiltrating T cell, a memory T cell, a naive T cell, and a NKT cell.

Examples of the method for generating the IL-7×CCL19-expressing immunocompetent cell according to certain aspect(s) of the present invention can include a generation method which involves introducing the expression vector according to certain aspect(s) of the present invention mentioned later to an immunocompetent cell. Alternative examples thereof can include a generation method which involves introducing a vector for the expression of a cell surface molecule specifically recognizing a cancer antigen, interleukin 7 (IL-7), and/or CCL19 to a fertilized egg, an ES cell, or an iPS cell, and then inducing the expression, and a generation method which involves further introducing, if necessary, a vector for the expression of a cell surface molecule specifically recognizing a cancer antigen, interleukin 7 (IL-7), and/or CCL19, to an immunocompetent cell obtained by separation from a transgenic mammal expressing the cell surface molecule specifically recognizing a cancer antigen by gene transfection.

Examples of the generation method which involves introducing the expression vector according to certain aspect(s) of the present invention mentioned later to the immunocompetent cell can include, but are not particularly limited to, an introduction method by a method known in the art, such as a viral infection method, a calcium phosphate method, lipofection, microinjection, and electroporation and can preferably include an introduction method by a viral infection method.

Examples of the viral infection method can include a method which involves transfecting a packaging cell such as a GP2-293 cell (manufactured by Takara Bio Inc.), a Plat-GP cell (manufactured by Cosmo Bio Co., Ltd.), a PG13 cell (ATCC CRL-10686), or a PA317 cell (ATCC CRL-9078) with the expression vector according to certain aspect(s) of the present invention and a packaging plasmid to generate a recombinant virus and infecting an immunocompetent cell with the recombinant virus. The viral infection method may be performed using a commercially available kit such as Retrovirus packaging Kit Eco (manufactured by Takara Bio Inc.).

The immunocompetent cell according to certain aspect(s) of the present invention may be generated by integrating a polynucleotide comprising nucleotide sequences encoding a cell surface molecule specifically recognizing a cancer antigen, IL-7, and CCL19 into the genome of a cell by use of a gene editing technique known in the art such the promoter can be expressed under the control of an appropriate promoter. Examples of the gene editing technique known in the art include a technique using endonuclease such as zinc finger nuclease, TALEN (transcription activator-like effector nuclease), CRISPR (clustered regularly interspaced short palindromic repeat)-Cas system. In the case of allowing the immunocompetent cell according to certain aspect(s) of the present invention to express an additional foreign protein, a polynucleotide comprising a nucleotide sequence encoding the additional foreign protein may be similarly integrated into the genome of the cell by use of the gene editing technique such that the promoter can be expressed under the control of an appropriate promoter. Examples of the method for integrating the polynucleotide into the genome of the cell such that the promoter can be expressed under the control of an appropriate promoter include: a method which involves integrating a polynucleotide in which nucleotide sequences encoding a cell surface molecule specifically recognizing a cancer antigen, IL-7, and CCL19 (or an additional protein) are functionally linked downstream of an appropriate promoter (i.e., a polynucleotide in which coding sequences are linked such that the factors (or the additional protein) can be expressed under the control of the promoter) into a noncoding region or the like of the cell genome; and a method which involves integrating a polynucleotide comprising nucleotide sequences encoding a cell surface molecule specifically recognizing a cancer antigen, IL-7, and CCL19 (or an additional protein) downstream of an endogenous promoter of the cell genome. Examples of the endogenous promoter include TCRα and TCRβ promoters.

The IL-7×CCL19-expressing immunocompetent cell according to certain aspect(s) of the present invention may also be allowed to express herpes simplex virus thymidine kinase (HSV-TK) or inducible caspase 9 mentioned later.

The IL-7×CCL19-expressing immunocompetent cell according to certain aspect(s) of the present invention expresses a cell surface molecule specifically recognizing a cancer antigen, IL-7, and CCL19. Therefore, The IL-7×CCL19-expressing immunocompetent cell according to certain aspect(s) of the present invention has high proliferative potential, survival capacity, and the ability to accumulate an intrinsic T cell and is applicable to adoptive immunotherapy using various immunocompetent cells. Examples of the adoptive immunotherapy include, but are not limited to, dendritic cell therapy, NK cell therapy, gamma-delta T cell therapy, alpha-beta T cell therapy, CTL therapy, and TIL therapy. An exemplary method can involve introducing the expression vector according to certain aspect(s) of the present invention mentioned later to an immunocompetent cell collected from a patient, amplifying the immunocompetent cell, and administering the immunocompetent cell to the patient. Hereinafter, specific examples will be given, though the present invention is not limited thereby. The dendritic cell therapy comprises the step of taking up a surgically extracted cancer tissue or a lysate thereof into a dendritic cell differentiated from a monocyte collected from a patient, and administering the dendritic cell into the body of the patient and may comprise the step of introducing the expression vector according to certain aspect(s) of the present invention into the dendritic cell. In this context, an epitope peptide of a cancer antigen molecule can also be artificially synthesized and used instead of the cancer tissue or the lysate. The NK cell therapy comprises the step of treating a lymphocyte collected from a patient with a plurality of stimulatory substances such as IL-2 to activate and amplify a NK cell, which is then administered to the patient and may comprise the step of introducing the vector according to certain aspect(s) of the present invention to the NK cell. Combined use of an antibody drug against cancer with the activated NK cell can be expected to produce an effect of efficiently attacking a cancer cell. The gamma-delta T cell therapy comprises the step of culturing and stimulating a lymphocyte collected from a patient using IL-2 or zoledronic acid to amplify a gamma-delta T cell, which is then administered to the patient and may comprise the step of introducing the expression vector according to certain aspect(s) of the present invention to the gamma-delta T cell. The alpha-beta T cell therapy comprises the step of culturing a lymphocyte harvested from a patient with an anti-CD3 antibody or IL-2 and administering an activated alpha-beta T cell to the patient and may comprise the step of introducing the expression vector according to certain aspect(s) of the present invention to the alpha-beta T cell. The CTL therapy comprises the step of stimulating a lymphocyte harvested from a patient with a cancer cell collected from the patient, culturing the lymphocyte by the addition of an anti-CD3 antibody or IL-2 to amplify CTL specific for the cancer cell, which is then administered to the patient and may comprise the step of introducing the expression vector according to certain aspect(s) of the present invention to the CTL. An antigen-presenting cell that presents a cancer antigen epitope peptide can also be used instead of the cancer cell. The TIL therapy comprises the step of harvesting a lymphocyte from a cancer tissue collected from a patient, stimulating and culturing the lymphocyte with IL-2 or the like, and administering the lymphocyte to the patient and may comprise the step of introducing the expression vector according to certain aspect(s) of the present invention to the lymphocyte.

The expression vector according to certain aspect(s) of the present invention is any of the following expression vectors (a) to (e) for generating the IL-7×CCL19-expressing immunocompetent cell according to certain aspect(s) of the present invention:
(a) an expression vector containing a nucleic acid encoding a cell surface molecule specifically recognizing a cancer antigen, a nucleic acid encoding IL-7, and a nucleic acid encoding CCL19;
(b) the following two expression vectors (b-1) and (b-2):
   (b-1) an expression vector containing a nucleic acid encoding a cell surface molecule specifically recognizing a cancer antigen; and
   (b-2) an expression vector containing a nucleic acid encoding IL-7 and a nucleic acid encoding CCL19;
(c) the following two expression vectors (c-1) and (c-2):
   (c-1) an expression vector containing a nucleic acid encoding a cell surface molecule specifically recognizing a cancer antigen, and a nucleic acid encoding IL-7; and
   (c-2) an expression vector containing a nucleic acid encoding CCL19;
(d) the following two expression vectors (d-1) and (d-2):
   (d-1) an expression vector containing a nucleic acid encoding IL-7; and
   (d-2) an expression vector containing a nucleic acid encoding a cell surface molecule specifically recognizing a cancer antigen, and a nucleic acid encoding CCL19; and
(e) the following three expression vectors (e-1), (e-2) and (e-3):
   (e-1) an expression vector containing a nucleic acid encoding a cell surface molecule specifically recognizing a cancer antigen;
   (e-2) an expression vector containing a nucleic acid encoding IL-7; and
   (e-3) an expression vector containing a nucleic acid encoding CCL19.

The expression vector according to certain aspect(s) of the present invention may further contain nucleic acids encoding other regulatory factors of immune function such as IL-15, CCL21, IL-2, IL-4, IL-12, IL-13, IL-17, IL-18, IP-10, CCL4, Flt3L, interferon-gamma, MIP-1 alpha, GM-CSF, M-CSF, TGF-beta, TNF-alpha.

Examples of the nucleic acid encoding a cell surface molecule specifically recognizing a cancer antigen, the nucleic acid encoding interleukin 7 (IL-7), and the nucleic acid encoding CCL19 can include respective mammal-derived nucleic acids and can preferably include human-derived nucleic acids. The respective nucleic acids can be appropriately selected according to the type of the cell to which the expression vector according to certain aspect(s) of the present invention is introduced. Sequence information on these respective nucleic acids can be appropriately obtained by the search of a document known in the art or a database such as NCBI (ncbi.nlm.nih.gov/guide/)

Examples of the nucleic acid encoding a cell surface molecule specifically recognizing a cancer antigen can preferably include a human-derived nucleic acid. Examples of such a nucleic acid encoding a cell surface molecule specifically recognizing a cancer antigen can include a nucleic acid encoding T cell receptor (TCR) or a nucleic acid encoding chimeric antigen receptor (CAR). This nucleic acid may be a naturally derived nucleic acid or may be an artificially synthesized nucleic acid and can be appropriately selected according to the type of the cell to which the expression vector according to certain aspect(s) of the present invention is introduced. Sequence information thereon can be appropriately obtained by the search of a document known in the art or a database such as NCBI (ncbi.nlm.nih.gov/guide/).

The nucleic acid encoding a cell surface molecule specifically recognizing a cancer antigen, the nucleic acid encoding IL-7 and the nucleic acid encoding CCL19 can be generated by a technique known in the art, such as a chemical synthesis method or a PCR amplification method, based on information on the nucleotide sequence of each encoding nucleic acid. Selected codons for encoding amino acids may be modified in order to optimize nucleic acid expression in a target host cell.

TCR for the nucleic acid encoding TCR may be a heterodimer consisting of an alpha chain and a beta chain (alpha-beta TCR) or may be a heterodimer consisting of a gamma chain and a delta chain (gamma-delta TCR). The nucleic acid encoding alpha-beta TCR comprises both of a nucleic acid encoding the alpha chain of TCR and a nucleic acid encoding the beta chain thereof. The nucleic acid encoding gamma-delta TCR includes both of a nucleic acid encoding the gamma chain of TCR and a nucleic acid encoding the delta chain thereof.

Sequence information on the nucleic acid encoding TCR can be identified from the nucleic acids of the alpha chain and the beta chain as a TCR subunit of CTL induced using a particular antigenic peptide by use of a method known in the art (International Publication No. WO 2007/032255; and Morgan et al., J Immunol, 171, 3288 (2003)). For example, PCR is preferred for analyzing TCR. PCR primers for TCR analysis can be, for example, 5′-R primer (5′-gtctaccaggcattcgcttcat-3′: SEQ ID NO: 3) as a 5′ primer and 3-TRa-C primer (5′-tcagctggaccacagccgcagcgt-3′: SEQ ID NO: 4) specific for a TCR alpha chain C region, 3-TRb-C1 primer (5′-tcagaaatcctttctcttgac-3′: SEQ ID NO: 5) specific for a TCR beta chain C1 region, or 3-TRbeta-C2 primer (5′-ctagcctctggaatcctttctctt-3′: SEQ ID NO: 6) specific for a TCR beta chain C2 region as a 3′ primer, though the primers are not limited thereto. A TCR derivative can bind, with high binding activity, to a target cell presenting an antigenic peptide and can arbitrarily mediate in vivo and in vitro the efficient killing of the target cell presenting an antigenic peptide.

The nucleic acid encoding TCR is, for example, a nucleic acid encoding TCR such as MART1-specific TCR (Cancer Res. 54, 5265-5268 (1994)), MAGE-A3-specific TCR (Anticancer Res., 20, 1793-1799 (2000)), gp100-specific TCR (J. Immunol. 170, 2186-2194 (2003)), NY-ESO-1-specific TCR (J. Immunol., 174, 4415-4423 (2005)), WT1-specific TCR (Blood, 106, 470-476 (2005)), MAGE-A1-specific TCR (Int. Immunol., 8, 1463-1466 (1996)), or P1A-specific TCR (Sarma, S., Y. Guo, Y. Guilloux, C. Lee, X.-F. Bai, Y. Liu. 1999. Cytotoxic T lymphocytes to an unmutated tumor antigen P1A: normal development but restrained effector function. J. Exp. Med. 189: 811) and may be a nucleotide sequence having 80% or higher, preferably 85% or higher, more preferably 90% or higher, further preferably 95% or higher, most preferably 98% or higher identity to a nucleotide sequence encoding any of the TCRs described in the documents as long as the TCR can recognize the antigen molecule bound with a MHC molecule and can activate a T cell. In the nucleotide sequence encoding any of the TCRs described in the documents, a sequence encoding CDR is identified, and a nucleotide sequence that maintains the sequence encoding CDR and has a sequence, other than the sequence encoding CDR, having 60% or higher, preferably 70% or higher, more preferably 80% or higher, further preferably 90% or higher, most preferably 95% or higher identity to the nucleotide sequence encoding any of the TCRs described in the documents may be used.

Examples of the nucleic acid encoding IL-7 can include a nucleotide sequence encoding the amino acid sequence represented by SEQ ID NO: 1. The nucleic acid encoding IL-7 may be a nucleotide sequence having 80% or higher, preferably 85% or higher, more preferably 90% or higher, further preferably 95% or higher, most preferably 98% or higher identity to the nucleotide sequence encoding the amino acid sequence represented by SEQ ID NO: 1 as long as the IL-7 has a cell proliferation rate- or cell survival rate-enhancing effect. Examples of the nucleic acid encoding CCL19 can include a nucleotide sequence encoding the amino acid sequence represented by SEQ ID NO: 2. A nucleotide sequence having 80% or higher, preferably 85% or higher, more preferably 90% or higher, further preferably 95% or higher, most preferably 98% or higher identity to the nucleotide sequence encoding the amino acid sequence represented by SEQ ID NO: 2 may be used as long as the CCL19 has a chemoattractive effect on a cell.

The expression vector according to certain aspect(s) of the present invention may also contain a nucleic acid encoding a suicide gene. The suicide gene means a gene having a function of directly or secondarily inducing a substance having cytotoxicity and killing a cell expressing this suicide gene. The expression vector according to certain aspect(s) of the present invention containing the nucleic acid encoding a suicide gene can regulate an immunocompetent cell in vivo by the administration of a drug activating the function of the suicide gene according to the course of treatment of cancer, for example, when tumor has disappeared. IL-7 or CCL19, unlike other cytokines, is less likely to cause cytokine release syndrome or tumorigenic transformation of a transgenic cell as an adverse reaction. However, as a result of enhancing the function of an immunocompetent cell introduced with the expression vector according to certain aspect(s) of the present invention, a cytokine or the like released upon attack on a target cancer tissue may unexpectedly influence its surrounding tissues. In such a case, the expression vector according to certain aspect(s) of the present invention containing the nucleic acid encoding a suicide gene is capable of reliably reducing the risk of causing cytokine release syndrome.

Examples of the suicide gene can include genes encoding herpes simplex virus thymidine kinase (HSV-TK) and inducible caspase 9 described in documents given below. Examples of the drugs activating the function of these genes can include ganciclovir for the former and a CID (chemical induction of dimerization) compound AP1903 for the latter (Cooper L J., et al., Cytotherapy. 2006; 8 (2) 105-17; Jensen M. C. et al., Biol Blood Marrow Transplant. 2010 September; 16 (9): 1245-56; Jones B S. Front Pharmacol. 2014 Nov. 27; 5: 254; Minagawa K., Pharmaceuticals (Basel). 2015 May 8; 8 (2): 230-49; and Bole-Richard E., Front Pharmacol. 2015 Aug. 25; 6: 174).

In the expression vector (a) containing a nucleic acid encoding a cell surface molecule specifically recognizing a cancer antigen, a nucleic acid encoding IL-7, and a nucleic acid encoding CCL19 as the vector according to certain aspect(s) of the present invention, any of the nucleic acids may be arranged upstream or downstream of any of the nucleic acids. Specifically, the case of containing a nucleic acid encoding TCR as the nucleic acid encoding a cell surface molecule specifically recognizing a cancer antigen will be taken as an example. The expression vector may have the nucleic acid encoding TCR, the nucleic acid encoding IL-7 and the nucleic acid encoding CCL19, may have the nucleic acid encoding TCR, the nucleic acid encoding CCL19 and the nucleic acid encoding IL-7, may have the nucleic acid encoding IL-7, the nucleic acid encoding CCL19 and the nucleic acid encoding TCR, may have the nucleic acid encoding IL-7, the nucleic acid encoding TCR and the nucleic acid encoding CCL19, may have the nucleic acid encoding CCL19, the nucleic acid encoding TCR and the nucleic acid encoding IL-7, or may have the nucleic acid encoding CCL19, the nucleic acid encoding IL-7 and the nucleic acid encoding TCR, in order from upstream.

In the expression vector (b-2) containing a nucleic acid encoding IL-7 and a nucleic acid encoding CCL19 as the vector according to certain aspect(s) of the present invention, the arrangement of the nucleic acid encoding IL-7 and the nucleic acid encoding CCL19 is not particularly limited, and the nucleic acid encoding CCL19 may be arranged upstream or downstream of the nucleic acid encoding IL-7.

In the expression vector (c-1) containing a nucleic acid encoding a cell surface molecule specifically recognizing a cancer antigen, and a nucleic acid encoding IL-7 as the vector according to certain aspect(s) of the present invention, the arrangement of the nucleic acid encoding a cell surface molecule specifically recognizing a cancer antigen and the nucleic acid encoding IL-7 is not particularly limited, and the nucleic acid encoding IL-7 may be arranged upstream or downstream of the nucleic acid encoding a cell surface molecule specifically recognizing a cancer antigen.

In the expression vector (d-2) containing a nucleic acid encoding a cell surface molecule specifically recognizing a cancer antigen, and a nucleic acid encoding CCL19 as the vector according to certain aspect(s) of the present invention, the arrangement of the nucleic acid encoding a cell surface molecule specifically recognizing a cancer antigen and the nucleic acid encoding CCL19 is not particularly limited, and the nucleic acid encoding CCL19 may be arranged upstream or downstream of the nucleic acid encoding a cell surface molecule specifically recognizing a cancer antigen.

The nucleic acid encoding a cell surface molecule specifically recognizing a cancer antigen, the nucleic acid encoding IL-7 and the nucleic acid encoding CCL19 may respectively be transcribed by different promoters or may be transcribed by one promoter using an internal ribozyme entry site (IRES) or self-cleaving 2A peptide.

The expression vector according to certain aspect(s) of the present invention may comprise an arbitrary nucleic acid between the nucleic acid encoding IL-7 and the nucleic acid encoding CCL19 in the case of transcribing these nucleic acids by one promoter using an internal ribozyme entry site (IRES) or self-cleaving 2A peptide, between the nucleic acid encoding a cell surface molecule specifically recognizing a cancer antigen and the nucleic acid encoding IL-7 and the nucleic acid encoding CCL19 in the case of comprising the nucleic acid encoding a cell surface molecule specifically recognizing a cancer antigen, between a nucleic acid encoding an alpha chain and a nucleic acid encoding a beta chain in the case of comprising a nucleic acid encoding alpha-beta TCR, or between a nucleic acid encoding a gamma chain and a nucleic acid encoding a delta chain in the case of comprising a nucleic acid encoding gamma-delta TCR, as long as each nucleic acid can be expressed. These nucleic acids are preferably linked via a sequence encoding a self-cleaving peptide (2A peptide) or IRES, preferably a sequence encoding 2A peptide. The linkage using this sequence enables the efficient expression of each nucleic acid.

In the case of containing a nucleic acid encoding a suicide gene, the position of the suicide gene is not particularly limited, and the suicide gene may be located, for example, via a sequence encoding 2A peptide or IRES, downstream of a promoter for the expression of the nucleic acid encoding a cell surface molecule specifically recognizing a cancer antigen, the nucleic acid encoding IL-7, or the nucleic acid encoding CCL19 and upstream or downstream of each of these nucleic acids, or may be located downstream of a different promoter.

The 2A peptide is a virus-derived self-cleaving peptide and is characterized in that G-P (position of 1 residue from the C terminus) in the amino acid sequence represented by SEQ ID NO: 7 is cleaved in the endoplasmic reticulum (Szymczak et al., Expert Opin. Biol. Ther. 5 (5): 627-638 (2005)). Therefore, nucleic acids incorporated to flank the 2A peptide are intracellularly expressed independently from each other.

The 2A peptide is preferably 2A peptide derived from picornavirus, rotavirus, insect virus, Aphthovirus, or Trypanosoma virus, more preferably picornavirus-derived 2A peptide (F2A) shown in SEQ ID NO: 8.

The vector for the expression vector according to certain aspect(s) of the present invention may be linear or circular and may be a non-viral vector such as a plasmid, a viral vector, or a vector based on a transposon. Such a vector may contain control sequences such as a promoter and a terminator, and a selective marker sequence such as a drug resistance gene or a reporter gene. The nucleic acid encoding IL-7 and the nucleic acid encoding CCL19 are operably located downstream of the promoter sequence so that each nucleic acid can be efficiently transcribed.

Examples of the promoter can include: a virus-derived promoter such as retrovirus LTR promoter, SV40 early promoter, cytomegalovirus promoter, herpes simplex virus thymidine kinase promoter; and a mammal-derived promoter such as phosphoglycerate kinase (PGK) promoter, Xist promoter, β-actin promoter, and RNA polymerase II promoter. Alternatively, tetracycline-responsive promoter which is induced by tetracycline, Mx1 promoter which is induced by interferon, or the like may be used. Use of the promoter which is induced by a particular substance in the expression vector according to certain aspect(s) of the present invention enables the regulation of induction of IL-7 and CCL19 expression in response to the course of treatment of cancer.

Examples of the viral vector can include a retrovirus vector, a lentivirus vector, an adenovirus vector, and an adeno-associated virus vector and can preferably include a retrovirus vector, more preferably a pMSGV vector (Tamada k et al., Clin Cancer Res 18: 6436-6445 (2002)) and a pMSCV vector (manufactured by Takara Bio Inc.). By use of a retrovirus vector, a transgene is integrated into the genome of a host cell and can therefore be expressed stably for a long period.

In order to confirm the containment of the expression vector according to certain aspect(s) of the present invention in the immunocompetent cell, for example, the expression of TCR can be examined by flow cytometry, Northern blotting, Southern blotting, PCR such as RT-PCR, ELISA, or Western blotting when the expression vector contains a nucleic acid encoding TCR, and the expression of a marker gene inserted in the expression vector according to certain aspect(s) of the present invention can be examined when the expression vector contains the marker gene.

When the expression vector contained in the IL-7×CCL19-expressing immunocompetent cell according to certain aspect(s) of the present invention contains a nucleic acid encoding TCR, the variable region of TCR to be expressed is extracellularly positioned. The TCR-expressing immunocompetent cell having this variable region of TCR is capable of recognizing the antigen molecule bound with a MHC molecule.

The anticancer agent according to certain aspect(s) of the present invention is not particularly limited as long as the anticancer agent comprises the IL-7×CCL19-expressing immunocompetent cell according to certain aspect(s) of the present invention and a pharmaceutically acceptable additive. Examples of the additive can include saline, buffered saline, a cell culture medium, dextrose, injectable water, glycerol, ethanol, and a combination thereof, a stabilizer, a solubilizer and a surfactant, a buffer and an antiseptic, a tonicity agent, a filler, and a lubricant.

The anticancer agent according to certain aspect(s) of the present invention can be administered to a test subject in need of treatment of cancer by use of a method known to those skilled in the art. Examples of the administration method can include intravenous, intratumoral, intracutaneous, subcutaneous, intramuscular, intraperitoneal, intraarterial, intramedullary, intracardiac, intraarticular, intrasynovial, intracranial, intrathecal, and subarachnoidal (spinal fluid) injection.

The amount of the IL-7×CCL19-expressing immunocompetent cell according to certain aspect(s) of the present invention contained in the anticancer agent to be administered can be appropriately adjusted according to the type, position, and severity of cancer, the age, body weight, and condition of the test subject to receive treatment, etc. Examples thereof can preferably include 1×10⁴ to 1×10¹⁰ cells, preferably 1×10⁵ to 1×10⁹ cells, more preferably 5×10⁶ to 5×10⁸ cells, in a single dose.

In an exemplary method, the anticancer agent to be administered can be independently administered 4 times, 3 times, twice, or once a day, at a 1-day, 2-day, 3-day, 4-day, or 5-day interval, once a week, at a 7-day, 8-day, or 9-day interval, twice a week, once a month, or twice a month.

The cancer for the anticancer agent according to certain aspect(s) of the present invention or a method for treating cancer mentioned later may be solid cancer or blood cancer. Examples thereof can include: cancer such as adenocarcinoma, squamous cell cancer, adenosquamous cancer, undifferentiated cancer, large-cell cancer, small-cell cancer, skin cancer, breast cancer, prostate cancer, urinary bladder cancer, vaginal cancer, neck cancer, uterine cancer, liver cancer, kidney cancer, pancreatic cancer, spleen cancer, lung cancer, tracheal cancer, bronchial cancer, colon cancer, small intestine cancer, stomach cancer, esophageal cancer, gallbladder cancer, testis cancer, and ovary cancer; cancer of a bone tissue, a cartilage tissue, a fat tissue, a muscle tissue, a vascular tissue, and a hematopoietic tissue; sarcoma such as chondrosarcoma, Ewing's sarcoma, malignant hemangioendothelioma, malignant schwannoma, osteosarcoma, and soft tissue sarcoma; blastoma such as hepatoblastoma, medulloblastoma, nephroblastoma, neuroblastoma, pancreatoblastoma, pleuropulmonary blastoma, and retinoblastoma; embryonic cell tumor; lymphoma; and leukemia.

The anticancer agent according to certain aspect(s) of the present invention can be used in combination with an additional anticancer agent. Examples of the additional anticancer agent can include: an alkylating agent such as cyclophosphamide, bendamustine, ifosfamide, and dacarbazine; an antimetabolite such as pentostatin, fludarabine, cladribine, methotrexate, 5-fluorouracil, 6-mercaptopurine, and enocitabine; a molecular targeting drug such as rituximab, cetuximab, and trastuzumab; a kinase inhibitor such as imatinib, gefitinib, erlotinib, afatinib, dasatinib, sunitinib, and trametinib; a proteasome inhibitor such as bortezomib; a calcineurin inhibitor such as cyclosporine and tacrolimus; an anticancer antibiotic such as idarubicin, doxorubicin, and mitomycin C; a vegetable alkaloid such as irinotecan and etoposide; a platinum-containing drug such as cisplatin, oxaliplatin, and carboplatin; a hormone therapeutic such as tamoxifen and bicalutamide; and an immunoregulatory drug such as interferon, nivolumab, and pembrolizumab and can preferably include an alkylating agent and an antimetabolite.

Examples of the method for “using the anticancer agent according to certain aspect(s) of the present invention in combination with the additional anticancer agent” can include a method using the additional anticancer agent in the treatment, followed by use of the anticancer agent according to certain aspect(s) of the present invention, a method concurrently using the anticancer agent according to certain aspect(s) of the present invention and the additional anticancer agent, and a method using the anticancer agent according to certain aspect(s) of the present invention in the treatment, followed by use of the additional anticancer agent and can preferably include a method using the additional anticancer agent in the treatment, followed by use of the anticancer agent according to certain aspect(s) of the present invention. The combined use of the anticancer agent according to certain aspect(s) of the present invention and the additional anticancer agent further improves a therapeutic effect on cancer and can also reduce the adverse reaction of each anticancer agent by decreasing the administration frequency or dose of the anticancer agent. Also, the additional anticancer agent may be contained in the anticancer agent according to certain aspect(s) of the present invention.

Examples of alternative aspect 1 of the present invention can include 1) a method for treating cancer, comprising administering an immunocompetent cell expressing a cell surface molecule specifically recognizing a cancer antigen, interleukin 7 (IL-7), and CCL19 to a patient in need of treatment of cancer, 2) an immunocompetent cell expressing a cell surface molecule specifically recognizing a cancer antigen, interleukin 7 (IL-7), and CCL19, for use as an anticancer agent, and 3) use of an immunocompetent cell expressing a cell surface molecule specifically recognizing a cancer antigen, interleukin 7 (IL-7), and CCL19, for the preparation of an anticancer agent.

Examples of alternative aspect 2 of the present invention can include a kit for the generation of an immunocompetent cell expressing a cell surface molecule specifically recognizing a cancer antigen, interleukin 7 (IL-7), and CCL19, comprising the expression vector according to certain aspect(s) of the present invention. The kit is not particularly limited as long as the kit comprises the expression vector according to certain aspect(s) of the present invention. The kit may comprise an instruction manual for the generation of the IL-7×CCL19-expressing immunocompetent cell according to certain aspect(s) of the present invention, and a reagent for use in the introduction of the expression vector according to certain aspect(s) of the present invention to an immunocompetent cell.

### Example 1

### (Selection of Regulatory Factors of Immune Function)

At least several hundred different types of molecules that can regulate the function of T cells are present in vivo. The inventors first selected IL-7 and CCL19 from among an enormous number of combinations on the basis of the previous findings or experiments, as regulatory molecules for further enhancing the immune function-regulating effect of immunocompetent cells, and also selected the combination of these two molecules, i.e., the combination of IL-7 and CCL19, not each alone. The inventors generated a vector for the expression of these regulatory factors of immunocompetent cell immune function.

### (Generation of Expression Vector for Expression of IL-7 and CCL19-1)

An anti-FITC CAR DNA fragment (SEQ ID NO: 9) encoding anti-FITC CAR consisting of anti-FITC scFv, a mouse CD8 transmembrane region, and mouse CD28-4-1BB-CD3ζ intracellular signal motifs, a F2A-MCS DNA fragment (SEQ ID NO: 10) encoding 2A peptide (F2A) shown in SEQ ID NO: 8 and a multicloning site (MCS) following the peptide, and an IL-7-F2A-CCL19 DNA fragment (SEQ ID NO: 11) encoding mouse IL-7 (without a stop codon) and F2A and mouse CCL19 following the mouse IL-7 were artificially synthesized (Life Technologies Corp.).

In order to generate a vector for the expression of IL-7 and CCL19, the anti-FITC CAR DNA fragment and the F2A-MCS DNA fragment were linked to generate an anti-FITC CAR-F2A-MCS construct. Then, the generated construct was cloned into a pMSGV retrovirus expression vector (Tamada k et al., Clin Cancer Res 18: 6436-6445 (2002)) to generate a pMSGV vector containing anti-FITC CAR-F2A-MCS. The IL-7-F2A-CCL19 DNA fragment was inserted to the MCS of the pMSGV vector by restriction enzyme (NsiI and SalI) treatment and ligation to obtain a pMSGV vector containing anti-FITC CAR-F2A-IL-7-F2A-CCL19 (IL-7×CCL19 expression vector (1)). The map of the obtained vector is shown in FIG. 1. Also, the anti-FITC CAR DNA fragment was cloned into the pMSGV retrovirus expression vector to generate a pMSGV vector free from IL-7 and CCL19 as a control (control vector (1)).

(Generation of Retrovirus Introduced with IL-7×CCL19 Expression Vector) For the transduction of mouse T cells, retrovirus was generated. A GP2-293 packaging cell line (manufactured by Takara Bio Inc.) was transfected with the aforementioned IL-7×CCL19 expression vector (1) or control vector (1) and a pCL-Eco plasmid (manufactured by Imgenex Corp.) using Lipofectamine 2000 or 3000 (manufactured by Life Technologies Corp.) to generate retrovirus introduced with the IL-7×CCL19 expression vector (1) or the control vector (1).

DMEM supplemented with 10% FCS, 100 U/ml penicillin, and 100 mg/ml streptomycin was used as a culture medium for the GP2-293 cells. RPMI-1640 supplemented with 10% FCS, 100 U/ml penicillin, 100 mg/ml streptomycin, 50 mM 2-mercaptoethanol, and 2 mM L-glutamine was used as a culture medium for T cells used in Examples mentioned later.

### (Transduction of Mouse T Cells)

For the transduction of mouse T cells, 3×10⁶ purified mouse T cells derived from the spleen and lymph nodes were activated for 48 hours with immobilized anti-CD3 mAb (3 μg/ml) and IL-2 (100 IU/ml). Then, the supernatant containing the thus-generated retrovirus introduced with the IL-7×CCL19 expression vector (1) or the control vector (1) was mixed with the activated mouse T cells mentioned above (1×10⁶ cells/ml) in a plate coated with 25 μg/ml RetroNectin® (manufactured by Takara Bio Inc.). After centrifugation at 1500 rpm for 2 hours, the cells were cultured for 6 hours in the presence of IL-2 (100 IU/ml). In order to remove the retrovirus from the culture medium, the mouse T cells were recovered, transferred to a fresh growth culture medium (RPMI) containing IL-2 (100 IU/ml), and further cultured for 42 hours to obtain mouse T cells introduced with the IL-7×CCL19 expression vector (1) (IL-7/CCL19-expressing T cells (1)) or mouse T cells introduced with the control vector (1) (control T cells (1)).

### (Generation of Expression Vector for Expression of IL-7 and CCL19-2)

A pMSGV vector containing anti-human CD20 CAR-F2A-IL-7-F2A-CCL19 (IL-7×CCL19 expression vector (2)) was generated in the same way as in the preceding section “Generation of expression vector for expression of IL-7 and CCL19-1” except that in the generation of the IL-7×CCL19 expression vector (1) described above, the sequence of the anti-FITC scFv region contained in the sequence represented by SEQ ID NO: 9 was replaced with a sequence of anti-human CD20 scFv (SEQ ID NO: 12) synthesized by Life Technologies Corp. on the basis of the sequence of rituximab. Likewise, a pMSGV vector free from IL-7 and CCL19 (control vector (2)) was generated in the same way as in the preceding section “Generation of expression vector for expression of IL-7 and CCL19-1” except that in the generation of the control vector (1) described above, the sequence of the anti-FITC scFv region contained in the sequence represented by SEQ ID NO: 9 was replaced with the sequence of anti-human CD20 scFv (SEQ ID NO: 12). The IL-7×CCL19 expression vector (2) or the control vector (2) was transferred to mouse T cells using retrovirus in the same way as above to generate IL-7/CCL19-expressing T cells (2) or control T cells (2).

### Example 2

### (Cell Number and Viability of IL-7/CCL19-Expressing T Cells)

Study was conducted on whether or not IL-7 or CCL19 produced by the IL-7/CCL19-expressing T cells would exert biological function and exhibit an immunity-inducing effect. A sample containing the generated IL-7/CCL19-expressing T cells (2) (4×10⁵ cells) or the control T cells (2) was cultured for 5 days. The culture was performed without antigen stimulation with CD20 in order to eliminate the influence of human CD20 CAR on the expression of IL-7 and CCL19. Then, the cell number and the viability were examined using trypan blue. The results are shown in FIGS. 2A and 2B. FIG. 2A shows the cell number, and FIG. 2B shows the viability. The filled column shows the results about the IL-7/CCL19-expressing T cells, and the open column shows the results about the control T cells.

### (Results)

As shown in FIGS. 2A and 2B, the cell number and the viability of the IL-7/CCL19-expressing T cells (2) were approximately 5 times and approximately 2 times, respectively, higher than those of the control T cells (2). These results demonstrated that by use of the IL-7/CCL19-expressing T cells prepared by introducing the expression vector according to certain aspect(s) of the present invention to T cells, IL-7 or CCL19 exerts biological function and exhibits an immunity-inducing effect.

### Example 3

### [T Cell Migration Test]

### (T Cell Migration Test Using IL-7/CCL19-Expressing T Cells)

The chemoattractive effect of CCL19 was studied by a cell migration test using Transwell. The migration properties of responder T cells were measured by migration through a polycarbonate filter having a pore size of 5 μm using 96-well Transwell® chambers (Costar, manufactured by Corning, Inc.). Specifically, the IL-7/CCL19-expressing T cells (1) or the control T cells (1) were cultured in the lower chamber. The culture was performed without antigen stimulation with FITC in order to eliminate the influence of FITC CAR on the expression of IL-7 and CCL19. The responder T cells were prepared from the spleen or lymph nodes by negative selection using MACS (manufactured by Miltenyi Biotec GmbH). The responder T cells were labeled with CytoTell blue (manufactured by AAT Bioquest, Inc.) and cultured for 3 hours in the upper chamber. The migration from the upper chamber to the lower chamber was examined by flow cytometry (EC800; manufactured by Sony Corp.), and FlowJo software (manufactured by Tree Star, Inc.) was used in data analysis. The results are shown in FIG. 3. In FIG. 3, the filled column shows the results about the IL-7/CCL19-expressing T cells (1), the open column shows the results about the control T cells (1), and the ordinate shows the absolute number of responder T cells that migrated to the lower chamber. Statistically significant difference was studied by the Student's t-test.

### (Results)

As shown in FIG. 3, the IL-7/CCL19-expressing T cells (1) allowed T cells to migrate to the lower chamber by approximately 1.8 times as compared with the control T cells (1). In lymphocyte (e.g., T cell) transfer therapy, damage to cancer cells by administered T cells is important as a matter of course, and in addition, it is important to activate endogenous T cells (=host's immunocytes) originally present in a cancer patient and thereby recruit these cells as cells attacking the cancer cells. For this purpose, it is preferred not only to transfer lymphocytes having antitumor activity ab extra but to evoke the active interaction between the transferred T cells and the endogenous T cells by some approach so that the endogenous T cells are accumulated locally to cancer, from the viewpoint of enhancing immunotherapeutic effects. As seen from the results of FIG. 3, the IL-7/CCL19-expressing T cells (1) had the ability to accumulate intrinsic T cells, demonstrating that the active interaction between the transferred T cells and the endogenous T cells can be induced.

The results of FIGS. 2A, 2B, and 3 demonstrated that the T cells expressing IL-7 and CCL19 possess important effects, indispensable for the induction of immunity, of effectively proliferating by IL-7, having a high viability, and accumulating T cells via CCL19, and have an excellent immunity-inducing effect. In short, the expression of the two regulatory molecules, i.e., “IL-7” and “CCL19”, in immunocompetent cells was shown to enable improvement in the proliferative potential, the viability, and the immunity-inducing effect of the immunocompetent cells. Furthermore, as mentioned above, the T cells expressing IL-7 and CCL19 possess all of the proliferative potential, the survival capacity, and the ability to accumulate a T cell, suggesting the possibility that these T cells have an effect of infiltrating into cancer tissues on T cells or dendritic cells and a tumor growth inhibitory effect.

### Example 4

### [Generation of IL-7×CCL19×HSV-TK Expression Vector]

A nucleotide sequence in which nucleotide sequences encoding IL-7 gene, CCL19 gene and a suicide gene HSV-TK are arranged in tandem so as to flank a nucleotide sequence encoding a self-cleaving peptide 2A peptide can be cloned into the multicloning site of a pMSGV1 vector to generate a vector for the expression of IL-7, CCL19, and HSV-TK. The map of this vector is shown in FIG. 4.

Immunocompetent cells introduced with the IL-7×CCL19×HSV-TK expression vector thus generated are capable of regulating immunocompetent cells within a test subject by the administration of ganciclovir to the test subject given the immunocompetent cells.

### Example 5

### [Generation of TCR×IL-7×CCL19 Expression Vector]

A nucleotide sequence in which nucleotide sequences encoding TCR gene, IL-7 gene and CCL19 gene are arranged in tandem so as to flank a nucleotide sequence encoding a self-cleaving peptide 2A peptide can be cloned into the multicloning site of a pMSGV1 vector to generate a vector for the expression of TCR, IL-7 and CCL19. The map of this vector is shown in FIG. 5.

Immunocompetent cells introduced with the TCR×IL-7×CCL19 expression vector thus generated are capable of specifically binding to not only the cancer antigen present on cancer cell surface but a complex of a cancer antigen-derived peptide presented by MHC within cancer cells and are capable of inducing T cells specific for a wider range of tumor-associated target molecules.

### Example 6

[Generation of Expression Vector for Expression of IL-7, CCL19 and eGFP]

An IL-7-F2A-CCL19 DNA fragment encoding mouse IL-7 (without a stop codon) and F2A and mouse CCL19 following the mouse IL-7 was artificially synthesized (Life Technologies Corp.).

In order to generate a vector for the expression of IL-7, CCL19 and eGFP, the IL-7-F2A-CCL19 DNA fragment thus synthesized was inserted to the MCS of a pMSGV retrovirus expression vector having a F2A-eGFP sequence (Tamada k et al., Clin Cancer Res 18: 6436-6445 (2002)) by restriction enzyme (NcoI and EcoRI) treatment and ligation to obtain a pMSGV vector containing an IL-7-F2A-CCL19-F2A-eGFP DNA fragment (SEQ ID NO: 13) (IL-7×CCL19 expression vector (3)). The map of the obtained vector is shown in FIG. 6. Also, a pMSGV vector containing eGFP and containing neither IL-7 nor CCL19 (control vector (3)) was generated as a control. In SEQ ID NO: 13, nucleotide positions 1 to 462 represent a nucleic acid encoding IL-7 (nucleotide positions 1 to 75 represent a signal sequence of IL-7), nucleotide positions 463 to 537 represent a nucleic acid encoding F2A, nucleotide positions 538 to 861 represent a nucleic acid encoding CCL19 (nucleotide positions 538 to 612 represent a signal sequence of CCL19), nucleotide positions 868 to 942 represent a nucleic acid encoding F2A, nucleotide positions 946 to 1662 represent a nucleic acid encoding eGFP, and nucleotide positions 1663 to 1665 represent a stop codon. The amino acid sequence corresponding to the nucleotide sequence represented by SEQ ID NO: 13 is shown in SEQ ID NO: 14. In order to use a restriction enzyme NcoI, thymine (t) at nucleotide position 4 in SEQ ID NO: 13 was replaced with guanine (g) (phenylalanine (F) at amino acid position 2 in SEQ ID NO: 14 was replaced with valine (V)).

[Generation of T Cells Expressing P815 Tumor Antigen P1A-Specific TCR, IL-7, CCL19, and eGFP]

Spleen cells were collected from a transgenic mouse expressing H-2L^{d}-restricted TCR specific for P815 tumor antigen P1A (Sarma, S., Y. Guo, Y. Guilloux, C. Lee, X.-F. Bai, Y. Liu. 1999. J. Exp. Med. 189: 811) obtained from Y. Liu. Mouse T cells expressing P815 tumor antigen P1A-specific TCR derived from the spleen cells (P1A-specific TCR-T cells) were obtained. Then, retrovirus introduced with the IL-7×CCL19 expression vector (3) or the control vector (3) was generated in the same way as in Example 1. The cells activated with P1A peptide for 48 hours were transduced with the spleen cells (3×10⁶ cells/well) including the P1A-specific TCR-T cells to obtain P1A-specific TCR/IL-7/CCL19/eGFP-expressing T cells or P1A-specific TCR/eGFP-expressing T cells. The transduction with each expression vector was confirmed by flow cytometry analysis of detecting eGFP as a surrogate marker. The respective eGFP expression levels of the obtained T cells were 70 to 80% in all experiments.

On day 0, 5×10⁵ cells of P815 mastocytoma suspended in 0.1 ml of HBSS were subcutaneously inoculated to the flank of each 6- to 10-week-old male DBA/2 mouse (n=30). On day 6, the mice were irradiated at a sublethal dose (3 to 5 Gy) for preconditioning. On day 7, the mice were divided into 3 groups (n=10). The P1A-specific TCR/IL-7/CCL19/eGFP-expressing T cells or the P1A-specific TCR/eGFP-expressing T cells (both the cells were 70 to 80% eGFP-positive) were intravenously administered at 1×10⁶ cells to each mouse. Then, the survival rate of each mouse was analyzed while the tumor volumes of dead mice were measured. The results of analyzing the survival rate of each mouse are shown in FIG. 7, and the results of measuring the tumor volumes of dead mice are shown in FIG. 8.

In FIG. 7, ▴ represents the results about untreated mice, ▪ represents the results about the mice given the P1A-specific TCR/eGFP-expressing T cells, ● represents the results about the mice given the P1A-specific TCR/IL-7/CCL19/eGFP-expressing T cells, the abscissa represents the number of days (day) after subcutaneous inoculation of P815 mastocytoma, and the ordinate represents the survival rate (%). 80% of the mice given the P1A-specific TCR/IL-7/CCL19/eGFP-expressing T cells survived even on day 60, and 50% thereof survived even after 100 days. Thus, use of the immunocompetent cells expressing P1A-specific TCR, IL-7 and CCL19 was shown to exert an antitumor effect and suppress decrease in survival rate caused by tumor.

In FIG. 8, the abscissa represents the number of days (day) after subcutaneous inoculation of P815 mastocytoma, and the ordinate represents the tumor volume (mm³). As is evident from FIG. 8, increase in tumor volume was remarkably suppressed in the mice given the P1A-specific TCR/IL-7/CCL19/eGFP-expressing T cells, demonstrating that the P1A-specific TCR/IL-7/CCL19/eGFP-expressing T cells have excellent antitumor activity and exert a therapeutic effect on solid cancer.

The immunocompetent cell according to certain aspect(s) of the present invention can be any immunocompetent cell that expresses a cell surface molecule specifically recognizing human mesothelin, IL-7, and CCL19, and is preferably an immunocompetent cell containing an exogenous nucleic acid encoding cell surface molecule, an exogenous nucleic acid encoding IL-7, and an exogenous nucleic acid encoding CCL19. This immunocompetent cell is capable of suppressing tumor formation ascribable to cancer cells expressing human mesothelin.

### (Human Mesothelin)

Human mesothelin, a 40 kDa protein, is rarely expressed in normal cells and highly expressed in cancer (e.g., mesothelioma and pancreatic cancer) cells. Sequence information on human mesothelin can be appropriately obtained by the search of a publicly known document or a database such as NCBI (http://www.ncbi.nlm.nih.gov/guide/). Examples of the amino acid sequence information on human mesothelin can include GenBank accession No. NP_037536.2, AAV87530.1, and their isoforms.

### (Cell Surface Molecule)

Examples of the cell surface molecule specifically recognizing human mesothelin can include a molecule or a factor providing specific identifiability to human mesothelin through expression on cell surface, such as CAR specifically recognizing human mesothelin, T cell receptor (TCR) specifically recognizing a peptide derived from human mesothelin, and a protein or a nucleic acid specifically binding to human mesothelin. The CAR is an artificial chimeric protein in which a single chain antibody (scFv) recognizing a cell surface antigen on cancer cells is fused with a signaling region that induces the activation of T cells.

The cell surface molecule is preferably localized on the cell surface of the immunocompetent cell through a signal peptide (leader sequence). Examples of the signal peptide can include polypeptides of an immune globulin heavy chain, an immunoglobulin light chain, CD8, T cell receptor α and β chains, CD3ζ, CD28, CD3ε, CD45, CD4, CD5, CD8, CD9, CD16, CD22, CD33, CD37, CD64, CD80, CD86, CD134, CD137, ICOS, CD154, and a GITR-derived signal peptide (leader sequence). Specific examples thereof can include a polypeptide that consists of an amino acid sequence having 85% or higher, preferably 90% or higher, more preferably 95% or higher, further preferably 98% or higher sequence identity to the amino acid sequence shown by SEQ ID NO: 25 or 26, and has action equivalent to that of the amino acid sequence of SEQ ID NO: 25 or 26, and a polypeptide that consists of an amino acid sequence derived from the amino acid sequence shown by SEQ ID NO: 25 or 26 by the deletion, substitution, insertion, and/or addition of one or several amino acids, and has action equivalent to that of the amino acid sequence shown by SEQ ID NO: 25 or 26. The signal peptide has been removed in a mature protein after the completion of localization.

In the present specification, the “amino acid sequence derived by the deletion, substitution, insertion, and/or addition of one or several amino acid residues” encompasses even an amino acid sequence with amino acid residues deleted, substituted, inserted, and/or added, for example, within the range of 1 to 30 residues, preferably within the range of 1 to 20 residues, more preferably within the range of 1 to 15 residues, further preferably within the range of 1 to 10 residues, further preferably within the range of 1 to 5 residues, further preferably within the range of 1 to 3 residues, further preferably within the range of 1 to 2 residues. These variation treatments of the amino acid residues can be performed by an arbitrary method known to those skilled in the art, such as chemical synthesis, a genetic engineering approach, or mutagenesis.

In the present specification, the term “identity” means the degree of polypeptide or polynucleotide sequence similarity (which is determined by the matching between a query sequence and another sequence (nucleic acid or protein sequence), preferably of the same type thereas). Preferred examples of the computer program method for calculating and determining the “identity” can include GCG BLAST (Basic Local Alignment Search Tool) (Altschul et al., J. Mol. Biol. 1990, 215: 403-410; Altschul et al., Nucleic Acids Res. 1997, 25: 3389-3402; and Devereux et al., Nucleic Acid Res. 1984, 12: 387), BLASTN 2.0 (Gish W., http://blast.Wustl.edu, 1996-2002), FASTA (Pearson and Lipman, Proc. Natl. Acad. Sci. USA 1988, 85: 2444-2448), and GCG GelMerge which determines and aligns a pair of contigs with the longest overlap (Wilbur and Lipman, SIAM J. Appl. Math. 1984, 44: 557-567; and Needleman and Wunsch, J. Mol. Biol. 1970, 48: 443-453).

### (Single Chain Antibody Specifically Recognizing Human Mesothelin)

When the cell surface molecule is CAR, a single chain antibody (scFv) specifically recognizing human mesothelin is preferably contained as a molecule specifically recognizing human mesothelin. In the single chain antibody specifically recognizing human mesothelin, the heavy chain variable region (VH) and the light chain variable region (VL) of an antibody specifically recognizing human mesothelin can be connected through a peptide linker for linking the heavy chain variable region and the light chain variable region. Examples of the combination of the heavy chain variable region and the light chain variable region in the single chain antibody specifically recognizing human mesothelin can include a combination given below. The light chain variable region may be positioned upstream (on the N-terminal side) or downstream (on the C-terminal side) of the heavy chain variable region.
(1-1) A combination of a heavy chain variable region comprising heavy chain CDR (complementarity determining region) 1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 29, and a light chain variable region comprising light chain CDR1 consisting of the amino acid sequence shown by SEQ ID NO: 30, light chain CDR2 consisting of the amino acid sequence shown by SEQ ID NO: 31, and light chain CDR3 consisting of the amino acid sequence shown by SEQ ID NO: 32;
(2-1) a combination of a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 29, and a light chain variable region comprising light chain CDR1 consisting of the amino acid sequence shown by SEQ ID NO: 33, light chain CDR2 consisting of the amino acid sequence shown by SEQ ID NO: 31, and light chain CDR3 consisting of the amino acid sequence shown by SEQ ID NO: 32; and
(3-1) a combination of a heavy chain variable region comprising heavy chain CDR1 consisting of the amino acid sequence shown by SEQ ID NO: 34, heavy chain CDR2 consisting of the amino acid sequence shown by SEQ ID NO: 35, and heavy chain CDR3 consisting of the amino acid sequence shown by SEQ ID NO: 36, and a light chain variable region comprising light chain CDR1 consisting of the amino acid sequence shown by SEQ ID NO: 37, light chain CDR2 consisting of the amino acid sequence shown by SEQ ID NO: 38, and light chain CDR3 consisting of the amino acid sequence shown by SEQ ID NO: 39.

In addition, CDRs of a heavy chain variable region and a light chain variable region specifically recognizing human mesothelin are identified according to the numbering system of IMGT, Kabat, Chothia, North, or Contact, etc. on the basis of the amino acid sequences of the heavy chain variable region and the light chain variable region of a publicly known antibody specifically recognizing human mesothelin, described in, for example, the following document (U.S. Pat. No. 8,357,783 and Japanese unexamined Patent Application Publication (Translation of PCT Application) No. 2017-518053). Examples of the combination can also include a combination of a heavy chain variable region and a light chain variable region having such CDRs. The CDRs can be identified from the following AbodyBuilder website (http://opig.stats.ox.ac.uk/webapps/sabdab-sabpred/Modelling.php).

Examples of the combination of the heavy chain variable region and the light chain variable region in the single chain antibody specifically recognizing human mesothelin can also include the following combination:
(1-2) a combination of a heavy chain variable region consisting of an amino acid sequence having 85% or higher, preferably 90% or higher, more preferably 95% or higher, further preferably 98% or higher sequence identity to the amino acid sequence shown by SEQ ID NO: 15, and a light chain variable region consisting of an amino acid sequence having 85% or higher, preferably 90% or higher, more preferably 95% or higher, further preferably 98% or higher sequence identity to the amino acid sequence shown by SEQ ID NO: 2;
(2-2) a combination of a heavy chain variable region consisting of an amino acid sequence having 85% or higher, preferably 90% or higher, more preferably 95% or higher, further preferably 98% or higher sequence identity to the amino acid sequence shown by SEQ ID NO: 17, and a light chain variable region consisting of an amino acid sequence having 85% or higher, preferably 90% or higher, more preferably 95% or higher, further preferably 98% or higher sequence identity to the amino acid sequence shown by SEQ ID NO: 4;
(3-2) a combination of a heavy chain variable region consisting of an amino acid sequence having 85% or higher, preferably 90% or higher, more preferably 95% or higher, further preferably 98% or higher sequence identity to the amino acid sequence shown by SEQ ID NO: 19, and a light chain variable region consisting of an amino acid sequence having 85% or higher, preferably 90% or higher, more preferably 95% or higher, further preferably 98% or higher sequence identity to the amino acid sequence shown by SEQ ID NO: 20;
(4-2) a combination of a heavy chain variable region consisting of an amino acid sequence having 85% or higher, preferably 90% or higher, more preferably 95% or higher, further preferably 98% or higher sequence identity to the amino acid sequence shown by SEQ ID NO: 15, and a light chain variable region consisting of an amino acid sequence having 85% or higher, preferably 90% or higher, more preferably 95% or higher, further preferably 98% or higher sequence identity to the amino acid sequence shown by SEQ ID NO: 18; and
(5-2) a combination of a heavy chain variable region consisting of an amino acid sequence having 85% or higher, preferably 90% or higher, more preferably 95% or higher, further preferably 98% or higher sequence identity to the amino acid sequence shown by SEQ ID NO: 17, and a light chain variable region consisting of an amino acid sequence having 85% or higher, preferably 90% or higher, more preferably 95% or higher, further preferably 98% or higher sequence identity to the amino acid sequence shown by SEQ ID NO: 2.

In addition, the combination may be a combination of a heavy chain variable region consisting of an amino acid sequence having 85% or higher, preferably 90% or higher, more preferably 95% or higher, further preferably 98% or higher sequence identity to the heavy chain variable region of a publicly known antibody specifically recognizing human mesothelin, described in, for example, the following document (U.S. Pat. No. 8,357,783 and Japanese unexamined Patent Application Publication (Translation of PCT Application) No. 2017-518053), and a light chain variable region consisting of an amino acid sequence having 85% or higher, preferably 90% or higher, more preferably 95% or higher, further preferably 98% or higher sequence identity to the light chain variable region of the publicly known antibody specifically recognizing human mesothelin.

Further examples of the combination of the heavy chain variable region and the light chain variable region in the single chain antibody specifically recognizing human mesothelin can also include the following combination:
(1-3) a combination of a heavy chain variable region consisting of the amino acid sequence shown by SEQ ID NO: 15, and a light chain variable region consisting of the amino acid sequence shown by SEQ ID NO: 2;
(2-3) a combination of a heavy chain variable region consisting of the amino acid sequence shown by SEQ ID NO: 17, and a light chain variable region consisting of the amino acid sequence shown by SEQ ID NO: 18;
(3-3) a combination of a heavy chain variable region consisting of the amino acid sequence shown by SEQ ID NO: 19, and a light chain variable region consisting of the amino acid sequence shown by SEQ ID NO: 20;
(4-3) a combination of a heavy chain variable region consisting of the amino acid sequence shown by SEQ ID NO: 15, and a light chain variable region consisting of the amino acid sequence shown by SEQ ID NO: 18; and
(5-3) a combination of a heavy chain variable region consisting of the amino acid sequence shown by SEQ ID NO: 17, and a light chain variable region consisting of the amino acid sequence shown by SEQ ID NO: 2.

In addition, the combination may be a combination of the heavy chain variable region and the light chain variable region of a publicly known antibody specifically recognizing human mesothelin, described in, for example, the following document (U.S. Pat. No. 8,357,783 and Japanese unexamined Patent Application Publication (Translation of PCT Application) No. 2017-518053).

### (Peptide Linker)

The heavy chain variable region and the light chain variable region are connected via a peptide linker. The length of the peptide linker is 2 to 30, preferably 15 to 25, more preferably 15, or 25. Specifically, preferred examples thereof can include a polypeptide that consists of an amino acid sequence having 85% or higher, preferably 90% or higher, more preferably 95% or higher, further preferably 98% or higher sequence identity to the amino acid sequence shown by SEQ ID NO: 40 or 41 comprising a glycine-serine continuous sequence, and has action equivalent to that of the amino acid sequence of SEQ ID NO: 40 or 41, and a polypeptide that consists of an amino acid sequence derived from the amino acid sequence shown by SEQ ID NO: 40 or41 by the deletion, substitution, insertion, and/or addition of one or several amino acids, and has action equivalent to that of the amino acid sequence shown by SEQ ID NO: 40 or 27.

Examples of the combination of the heavy chain variable region, the light chain variable region, and the peptide linker in the single chain antibody specifically recognizing human mesothelin can also include a combination given below. The term “sequentially” described below means in order from the N-terminal side.
(1-4) A combination sequentially comprising a heavy chain variable region consisting of the amino acid sequence shown by SEQ ID NO: 15, a peptide linker consisting of the amino acid sequence shown by SEQ ID NO: 40, and a light chain variable region consisting of the amino acid sequence shown by SEQ ID NO: 2;
(2-4) a combination sequentially comprising a heavy chain variable region consisting of the amino acid sequence shown by SEQ ID NO: 17, a peptide linker consisting of the amino acid sequence shown by SEQ ID NO: 40, and a light chain variable region consisting of the amino acid sequence shown by SEQ ID NO: 18;
(3-4) a combination sequentially comprising a light chain variable region consisting of the amino acid sequence shown by SEQ ID NO: 2, a peptide linker consisting of the amino acid sequence shown by SEQ ID NO: 40, and a heavy chain variable region consisting of the amino acid sequence shown by SEQ ID NO: 15;
(4-4) a combination sequentially comprising a heavy chain variable region consisting of the amino acid sequence shown by SEQ ID NO: 15, a peptide linker consisting of the amino acid sequence shown by SEQ ID NO: 41, and a light chain variable region consisting of the amino acid sequence shown by SEQ ID NO: 2;
(5-4) a combination sequentially comprising a light chain variable region consisting of the amino acid sequence shown by SEQ ID NO: 2, a peptide linker consisting of the amino acid sequence shown by SEQ ID NO: 41, and a heavy chain variable region consisting of the amino acid sequence shown by SEQ ID NO: 15;
(6-4) a combination sequentially comprising a heavy chain variable region consisting of the amino acid sequence shown by SEQ ID NO: 19, a peptide linker consisting of the amino acid sequence shown by SEQ ID NO: 40, and a light chain variable region consisting of the amino acid sequence shown by SEQ ID NO: 20;
(7-4) a combination sequentially comprising a light chain variable region consisting of the amino acid sequence shown by SEQ ID NO: 20, a peptide linker consisting of the amino acid sequence shown by SEQ ID NO: 40, and a heavy chain variable region consisting of the amino acid sequence shown by SEQ ID NO: 19;
(8-4) a combination sequentially comprising a heavy chain variable region consisting of the amino acid sequence shown by SEQ ID NO: 19, a peptide linker consisting of the amino acid sequence shown by SEQ ID NO: 41, and a light chain variable region consisting of the amino acid sequence shown by SEQ ID NO: 20; and
(9-4) a combination sequentially comprising a light chain variable region consisting of the amino acid sequence shown by SEQ ID NO: 20, a peptide linker consisting of the amino acid sequence shown by SEQ ID NO: 41, and a heavy chain variable region consisting of the amino acid sequence shown by SEQ ID NO: 19.

### (IL-7 and CCL19)

The IL-7 is a cytokine essential for the survival of T cells and is produced by non-hematopoietic cells such as stromal cells of the bone marrow, the thymus gland, or a lymphoid organ or tissue. On the other hand, T cells themselves are rarely found to have the ability to produce the IL-7.

The CCL19 is mainly produced from dendritic cells or macrophages of lymph nodes and has a function of initiating the migration of T cells, B cells, or mature dendritic cells via its receptor CCR7.

The organism from which the IL-7 or the CCL19 is derived is not particularly limited and is preferably a human. The amino acid sequences of these proteins are available from a publicly known sequence database such as GenBank. Examples of the amino acid sequence of human IL-7 can include a sequence registered under GenBank accession No: NM 000880.3 (SEQ ID NO: 42) and its isoform. Examples of the amino acid sequence of human CCL19 can include a sequence registered under GenBank accession No: NM 006274.2 (SEQ ID NO: 43) and its isoform. Although the IL-7 and the CCL19 may have a signal peptide, the signal peptide is removed in a mature protein. For example, in the amino acid sequence of human IL-7 described in SEQ ID NO: 42, a sequence from positions 1 to 25 corresponds to the signal peptide. For example, in the amino acid sequence of human CCL19 described in SEQ ID NO: 43, a sequence from positions 1 to 21 corresponds to the signal peptide.

The IL-7 or the CCL19 may be a variant of the natural protein as described above. Examples of the IL-7 variant can include a polypeptide that consists of an amino acid sequence having 85% or higher, preferably 90% or higher, more preferably 95% or higher, further preferably 98% or higher sequence identity to the amino acid sequence of human IL-7 described in SEQ ID NO: 42, and has action of enhancing a cell proliferation rate or a cell survival rate by IL-7, and a polypeptide that consists of an amino acid sequence derived from the amino acid sequence of human IL-7 described in SEQ ID NO: 42 by the deletion, substitution, insertion, and/or addition of one or several amino acids, and has action of enhancing a cell proliferation rate or a cell survival rate by IL-7. Examples of the human CCL19 variant can include a polypeptide that consists of an amino acid sequence having 85% or higher, preferably 90% or higher, more preferably 95% or higher, further preferably 98% or higher sequence identity to the amino acid sequence of human CCL19 described in SEQ ID NO: 43, and has the cell migrating action of CCL19, and a polypeptide that consists of an amino acid sequence derived from the amino acid sequence of human CCL19 described in SEQ ID NO: 43 by the deletion, substitution, insertion, and/or addition of one or several amino acids, and has the cell migrating action of CCL19.

### (Additional Immune Function Control Factor)

The immunocompetent cell according to certain aspect(s) of the present invention may further express an additional immune function control factor such as IL-15, CCL21, IL-2, IL-4, IL-12, IL-13, IL-17, IL-18, IP-10, interferon-γ, MIP-1alpha, GM-CSF, M-CSF, TGF-beta, or TNF-alpha. The additional immune function control factor is preferably an immune function control factor other than IL-12.

### (Transmembrane Region)

Examples of the transmembrane region according to the present invention can include polypeptides of transmembrane regions derived from CD8, T cell receptor α and β chains, CD3ζ, CD28, CD3ε, CD45, CD4, CD5, CD8, CD9, CD16, CD22, CD33, CD37, CD64, CD80, CD86, CD134, CD137, ICOS, CD154, and GITR. Preferred examples thereof can include a polypeptide that comprises an amino acid sequence having 85% or higher, preferably 90% or higher, more preferably 95% or higher, further preferably 98% or higher sequence identity to the amino acid sequence of a human CD8 transmembrane region shown by SEQ ID NO: 21, and has action equivalent to that of the amino acid sequence shown by SEQ ID NO: 21, and a polypeptide that consists of an amino acid sequence derived from the amino acid sequence shown by SEQ ID NO: 21 by the deletion, substitution, insertion, and/or addition of one or several amino acids, and has action equivalent to that of the amino acid sequence shown by SEQ ID NO: 21. CAR is anchored to the cell membranes of T cells through such a transmembrane region.

The transmembrane region may comprise a hinge region that consists of an arbitrary oligopeptide or polypeptide and has a length of 1 to 100 amino acids, preferably 10 to 70 amino acids. Examples of the hinge region can include the hinge region of human CD8.

### (Immunocompetent Cell Activating Signaling Region)

The immunocompetent cell activating signaling region is a region capable of transducing signals into the cell when the cell surface molecule recognizes mesothelin. The immunocompetent cell activating signaling region preferably comprises at least one or more members selected from intracellular regions of polypeptides of CD28, 4-1BB (CD137) GITR, CD27, OX40, HVEM, CD3ζ, or Fc receptor-associated γ chain, and more preferably three polypeptides, i.e., a polypeptide of a CD28 intracellular region, a polypeptide of a 4-1BB intracellular region, and a polypeptide of a CD3ζ intracellular region. Examples of the amino acid sequence of the CD28 intracellular region can include a polypeptide that comprises an amino acid sequence having 85% or higher, preferably 90% or higher, more preferably 95% or higher, further preferably 98% or higher sequence identity to the amino acid sequence shown by SEQ ID NO: 22, and has action equivalent to that of the amino acid sequence shown by SEQ ID NO: 22, and a polypeptide that consists of an amino acid sequence derived from the amino acid sequence shown by SEQ ID NO: 22 by the deletion, substitution, insertion, and/or addition of one or several amino acids, and has action equivalent to that of the amino acid sequence shown by SEQ ID NO: 22. Examples of the amino acid sequence of the 4-1BB intracellular region can include a polypeptide that comprises an amino acid sequence having 85% or higher, preferably 90% or higher, more preferably 95% or higher, further preferably 98% or higher sequence identity to the amino acid sequence shown by SEQ ID NO: 23, and has action equivalent to that of the amino acid sequence shown by SEQ ID NO: 23, and a polypeptide that consists of an amino acid sequence derived from the amino acid sequence shown by SEQ ID NO: 23 by the deletion, substitution, insertion, and/or addition of one or several amino acids, and has action equivalent to that of the amino acid sequence shown by SEQ ID NO: 23. Examples of the amino acid sequence of the CD3ζ intracellular region can include a polypeptide that comprises an amino acid sequence having 85% or higher, preferably 90% or higher, more preferably 95% or higher, further preferably 98% or higher sequence identity to the amino acid sequence shown by SEQ ID NO: 24, and has action equivalent to that of the amino acid sequence shown by SEQ ID NO: 24, and a polypeptide that consists of an amino acid sequence derived from the amino acid sequence shown by SEQ ID NO: 24 by the deletion, substitution, insertion, and/or addition of one or several amino acids, and has action equivalent to that of the amino acid sequence shown by SEQ ID NO: 24. When a T cell is used as the immunocompetent cell, a polypeptide capable of transducing signals into the T cell can be selected. When other immunocompetent cells are used, polypeptides capable of transducing signals into the immunocompetent cells can be selected. In the case of using a T cell as the immunocompetent cell, examples of the immunocompetent cell activating signaling region can include a polypeptide comprising the amino acid sequences shown by SEQ ID NOs: 8, 9 and 10 and can preferably include a polypeptide comprising the amino acid sequences shown by SEQ ID NOs: 8, 9 and 10 in order from the N-terminal side.

### (Extracellular Hinge Region and Spacer)

An extracellular hinge region consisting of an arbitrary oligopeptide or polypeptide may be located between the cell surface molecule recognizing mesothelin and the transmembrane region. Examples of the length of the extracellular hinge region can include 1 to 100 amino acid residues, preferably 10 to 70 amino acid residues. Examples of such an extracellular hinge region can include hinge regions derived from CD8, CD28, and CD4, and an immune globulin hinge region.

A spacer region consisting of an arbitrary oligopeptide or polypeptide may be located between the transmembrane region and the immunocompetent cell activating signaling region. Examples of the length of the spacer region can include 1 to 100 amino acid residues, preferably 10 to 50 amino acid residues. Examples of such a spacer region can include a glycine-serine continuous sequence.

### (Arrangement of Each Region)

In the CAR, each region described above can be arranged in order of the single chain antibody, the transmembrane region, and the immunocompetent cell activating signaling region from the N terminus. Specific examples thereof can include CAR in which the single chain antibody specifically recognizing human mesothelin, the extracellular hinge region of human CD8, the transmembrane region of human CD8, the T cell activating signaling region of human CD28, the T cell activating signaling region of human 4-1BB, and the T cell activating signaling region of human CD3ζ are arranged in order from the N-terminal side.

### (Protein to be Expressed by Suicide Gene)

The immunocompetent cell according to certain aspect(s) of the present invention may express a protein having a function of killing its own cell, such as herpes simplex virus thymidine kinase (HSV-TK) or inducible caspase 9 (protein to be expressed by a suicide gene). The expression of such a protein based on the suicide gene directly or secondarily induces a substance having cellular toxicity and can confer the function of killing its own cell. Hence, for example, the immunocompetent cell according to certain aspect(s) of the present invention present in a living body can be controlled by the administration of a drug activating the function after disappearance of tumor according to the course of treatment of cancer. Specifically, a risk of cytokine release syndrome can be reliably reduced, if necessary, for the immunocompetent cell according to certain aspect(s) of the present invention.

Examples of the drug activating the function of herpes simplex virus thymidine kinase (HSV-TK) or inducible caspase 9 can include ganciclovir for the former and AP1903 which is chemical induction of dimerization (CID) for the latter (Cooper L J., et al., Cytotherapy. 2006; 8 (2) 105-17; Jensen M. C. et al., Biol Blood Marrow Transplant. 2010 September; 16 (9): 1245-56; Jones B S. Front Pharmacol. 2014 Nov. 27; 5: 254; Minagawa K., Pharmaceuticals (Basel). 2015 May 8; 8 (2): 230-49; and Bole-Richard E., Front Pharmacol. 2015 Aug. 25; 6: 174).

### (Type of Cell for Immunocompetent Cell)

The type of the cell for the immunocompetent cell is not particularly limited as long as the cell is involved in immune response and can express the cell surface molecule specifically recognizing human mesothelin, the IL-7 and the CCL19 by the introduction of a nucleic acid encoding the cell surface molecule specifically recognizing human mesothelin, a nucleic acid encoding the IL-7, and a nucleic acid encoding the CCL19. The cell is preferably an immunocompetent cell separated from a living body. Examples thereof can include a lymphoid cell such as a T cell, a natural killer cell (NK cell), and a B cell, an antigen presenting cell such as a monocyte, a macrophage, and a dendritic cell, and a granulocyte such as a neutrophil, an eosinophil, a basophil, and a mast cell, separated from a living body. Specifically, preferred examples thereof can include a T cell derived or separated from a mammal such as a human, a dog, a cat, a pig, or a mouse, preferably a T cell derived or separated from a human. The T cell derived from a mammal such as a human, a dog, a cat, a pig, or a mouse includes a T cell obtained by artificially culturing ex vivo a T cell separated (collected) from the mammal such as a human, a dog, a cat, a pig, or a mouse, or a T cell subcultured from this T cell. The separated T cell can be a cell population mainly comprising T cells. Such a cell population may comprise additional cells other than T cells and preferably comprises T cells at a proportion of 50% or higher, preferably 60% or higher, more preferably 70% or higher, further preferably 80% or higher, most preferably 90% or higher. The T cell can be obtained by separating a cell population comprising the immunocompetent cell from a body fluid such as blood or bone marrow fluid, a tissue such as a spleen tissue, the thymus gland, or a lymph node, or immunocompetent cells infiltrating a cancer tissue such as primary tumor, metastatic tumor, or cancerous ascites. In order to elevate the proportion of T cells comprised in the cell population, the T cell may be further isolated or purified, if necessary, from the separated cell population by a standard method. A cell produced from an ES cell or an iPS cell may be utilized for the immunocompetent cell. Examples of such a T cell can include an alpha-beta T cell, a gamma-delta T cell, a CD8⁺ T cell, a CD4⁺ T cell, a tumor infiltrating T cell, a memory T cell, a naive T cell, and a NKT cell. The origin of the immunocompetent cell may be the same as or different from an administration subject. When the administration subject is a human, an autologous cell collected from a patient himself or herself as the administration subject may be used as the immunocompetent cell, or an allogeneic cell collected from another person may be used thereas. Specifically, the donor and the recipient may or may not be the same and are preferably the same.

### (Method for Producing Immunocompetent Cell)

Examples of the method for producing the immunocompetent cell according to certain aspect(s) of the present invention can include a production method of introducing a nucleic acid encoding a cell surface molecule, a nucleic acid encoding IL-7, and a nucleic acid encoding CCL19 to an immunocompetent cell, and can preferably include a production method of introducing the expression vector according to certain aspect(s) of the present invention mentioned later to an immunocompetent cell by a method described in, for example, Patent Document 1 or 2. Alternative examples thereof can include a method of purifying and obtaining an immunocompetent cell from a transgenic mammal produced by implanting a vector for expression of a cell surface molecule specifically recognizing human mesothelin, IL-7, and/or CCL19 into a fertilized egg, and a production method of further introducing, if necessary, the vector for expression of a cell surface molecule specifically recognizing human mesothelin, IL-7, and/or CCL19 to the immunocompetent cell purified and obtained from the transgenic mammal.

In the case of introducing a nucleic acid encoding a cell surface molecule, a nucleic acid encoding IL-7, and a nucleic acid encoding CCL19, or the vector according to certain aspect(s) of the present invention mentioned later to an immunocompetent cell, the method for introducing the nucleic acids or the vector can be any method for introducing the nucleic acids or the vector to the immunocompetent cell. Examples thereof can include a method such as an electroporation method (Cytotechnology, 3, 133 (1990)), a calcium phosphate method (Japanese unexamined Patent Application Publication No. 2-227075), a lipofection method (Proc. Natl. Acad. Sci. U.S.A., 84, 7413 (1987)), and a viral infection method. Examples of such a viral infection method can include a method of transfecting a packaging cell such as a GP2-293 cell (manufactured by Takara Bio Inc.), a Plat-GP cell (manufactured by Cosmo Bio Co., Ltd.), a PG13 cell (ATCC CRL-10686), or a PA317 cell (ATCC CRL-9078) with the vector to be introduced and a packaging plasmid to produce a recombinant virus, and infecting the immunocompetent cell with the recombinant virus (Patent Document 2).

In the case of producing the “immunocompetent cell that expresses a cell surface molecule specifically recognizing human mesothelin, IL-7, and CCL19” using a vector, the immunocompetent cell can be produced by any of the following methods:
(1) a method of introducing a vector for expression of a cell surface molecule specifically recognizing human mesothelin, IL-7 and CCL19, containing a nucleic acid encoding the cell surface molecule specifically recognizing human mesothelin, a nucleic acid encoding the IL-7 and a nucleic acid encoding the CCL19 to an immunocompetent cell;
(2) a method of introducing, at the same time or in stages, two types of vectors, i.e., a vector for expression of a cell surface molecule specifically recognizing human mesothelin, containing a nucleic acid encoding the cell surface molecule specifically recognizing human mesothelin, and a vector for expression of IL-7 and CCL19, containing a nucleic acid encoding the IL-7 and a nucleic acid encoding the CCL19, to an immunocompetent cell;
(3) a method of introducing, at the same time or in stages, two types of vectors, i.e., a vector for expression of a cell surface molecule specifically recognizing human mesothelin and IL-7, containing a nucleic acid encoding the cell surface molecule specifically recognizing human mesothelin and a nucleic acid encoding the IL-7, and a vector for expression of CCL19, containing a nucleic acid encoding the CCL19, to an immunocompetent cell;
(4) a method of introducing, at the same time or in stages, two types of vectors, i.e., a vector for expression of a cell surface molecule specifically recognizing human mesothelin and CCL19, containing a nucleic acid encoding the cell surface molecule specifically recognizing human mesothelin and a nucleic acid encoding the CCL19, and a vector for expression of IL-7, containing a nucleic acid encoding the IL-7, to an immunocompetent cell;
(5) a method of introducing, at the same time or in stages, two types of vectors, i.e., a vector for expression of a cell surface molecule specifically recognizing human mesothelin and IL-7, containing a nucleic acid encoding the cell surface molecule specifically recognizing human mesothelin and a nucleic acid encoding the IL-7, and a vector for expression of a cell surface molecule specifically recognizing human mesothelin and CCL19, containing a nucleic acid encoding the cell surface molecule specifically recognizing human mesothelin and a nucleic acid encoding the CCL19, to an immunocompetent cell;
(6) a method of introducing, at the same time or in stages, two types of vectors, i.e., a vector for expression of a cell surface molecule specifically recognizing human mesothelin and IL-7, containing a nucleic acid encoding the cell surface molecule specifically recognizing human mesothelin and a nucleic acid encoding the IL-7, and a vector for expression of IL-7 and CCL19, containing a nucleic acid encoding the IL-7 and a nucleic acid encoding the CCL19, to an immunocompetent cell;
(7) a method of introducing, at the same time or in stages, two types of vectors, i.e., a vector for expression of a cell surface molecule specifically recognizing human mesothelin and CCL19, containing a nucleic acid encoding the cell surface molecule specifically recognizing human mesothelin and a nucleic acid encoding the CCL19, and a vector for expression of IL-7 and CCL19, containing a nucleic acid encoding the IL-7 and a nucleic acid encoding the CCL19, to an immunocompetent cell; and
(8) a method of introducing, at the same time or in stages, three types of vectors, i.e., a vector for expression of a cell surface molecule specifically recognizing human mesothelin, containing a nucleic acid encoding the cell surface molecule specifically recognizing human mesothelin, a vector for expression of IL-7, containing a nucleic acid encoding the IL-7, and a vector for expression of CCL19, containing a nucleic acid encoding the CCL19, to an immunocompetent cell.

In the case of producing the “immunocompetent cell that expresses a cell surface molecule specifically recognizing human mesothelin, IL-7, and CCL19” using a vector, an immunocompetent cell that expresses the cell surface molecule specifically recognizing human mesothelin is prepared in advance, and the immunocompetent cell may be produced by any of the following methods using this immunocompetent cell that expresses the cell surface molecule specifically recognizing human mesothelin:
(1) a method of introducing a vector for expression of IL-7 and CCL19, containing a nucleic acid encoding the IL-7 and a nucleic acid encoding the CCL19 to the immunocompetent cell that expresses the cell surface molecule specifically recognizing human mesothelin; and
(2) a method of introducing, at the same time or in stages, two types of vectors, i.e., a vector for expression of IL-7, containing a nucleic acid encoding the IL-7, and a vector for expression of CCL19, containing a nucleic acid encoding the CCL19, to the immunocompetent cell that expresses the cell surface molecule specifically recognizing human mesothelin.

In the case of using each of the immunocompetent cells described above, cultures of the immunocompetent cell, containing the immunocompetent cell may be used. The nucleic acid encoding the cell surface molecule specifically recognizing human mesothelin, the nucleic acid encoding the IL-7, and the nucleic acid encoding the CCL19 may be integrated in the genome of the immunocompetent cell or may not be integrated in the genome (e.g., episomally), for use. In the case of using each of the immunocompetent cells described above, a mixture of an immunocompetent cell in which the nucleic acid encoding the cell surface molecule specifically recognizing human mesothelin, the nucleic acid encoding the IL-7, and the nucleic acid encoding the CCL19 are integrated in the genome of the immunocompetent cell, and an immunocompetent cell in which the nucleic acid encoding the cell surface molecule specifically recognizing human mesothelin, the nucleic acid encoding the IL-7, and the nucleic acid encoding the CCL19 are not integrated in the genome, may be used.

The “immunocompetent cell that expresses a cell surface molecule specifically recognizing human mesothelin, IL-7, and CCL19” as described above may be produced by integrating a nucleic acid encoding the cell surface molecule specifically recognizing human mesothelin, a nucleic acid encoding the IL-7, and a nucleic acid encoding the CCL19 in the genome of a cell so as to be expressible under the control of a suitable promoter by use of a publicly known gene editing technique. Examples of the publicly known gene editing technique include a technique using an endonuclease such as zing finger nuclease, TALEN (transcription activator-like effector nuclease), CRISPR (clustered regularly interspaced short palindromic repeat)-Cas system. In the case of allowing an immunocompetent cell that expresses, for example, CAR specifically recognizing human mesothelin (anti-human mesothelin CAR) to express an additional exogenous protein, a polynucleotide comprising a nucleotide sequence encoding the additional exogenous protein may similarly be integrated in the genome of the cell so as to be expressible under the control of a suitable promoter by use of the gene editing technique. Specific examples of such a method include: a method of integrating a polynucleotide comprising a nucleotide sequence encoding anti-human mesothelin CAR (or an additional protein), functionally linked to a suitable promoter to a non-coding region or the like of the cell genome; and a method of integrating a polynucleotide comprising a nucleotide sequence encoding anti-human mesothelin CAR (or an additional protein) to downstream of an endogenous promoter of the cell genome. Examples of the endogenous promoter include TCRα and TCRβ promoters.

### (Administration Subject)

Preferred examples of the administration subject can include a mammal and a mammalian cell. More preferred examples of such a mammal can include a human, a mouse, a dog, a rat, a guinea pig, a rabbit, a bird, sheep, a pig, cattle, a horse, a cat, a monkey, and a chimpanzee, particularly preferably a human.

### (Expression Vector)

The expression vector according to certain aspect(s) of the present invention can be any vector that is introduced into an immunocompetent cell or its precursor cell by contact with the cell so that a predetermined protein (polypeptide) encoded therein can be expressed in the immunocompetent cell to produce the immunocompetent cell according to certain aspect(s) of the present invention. The expression vector according to certain aspect(s) of the present invention is not particularly limited by an embodiment. Those skilled in the art are capable of designing and producing an expression vector that permits expression of the desired protein (polypeptide) in immunocompetent cells. Examples of the expression vector according to certain aspect(s) of the present invention comprising a nucleic acid encoding a cell surface molecule specifically recognizing human mesothelin, a nucleic acid encoding IL-7, and a nucleic acid encoding CCL19 can include any of expression vectors (f) to (n) given below for producing the immunocompetent cell according to certain aspect(s) of the present invention (hereinafter, also referred to as an “IL-7/CCL19 expression-anti-human mesothelin vector”). The term “two expression vectors” described below means a set of two types of expression vectors, and the term “three expression vectors” means a set of three types of expression vectors.
(f) An expression vector comprising a nucleic acid encoding a cell surface molecule specifically recognizing human mesothelin, a nucleic acid encoding IL-7, and a nucleic acid encoding CCL19;
(g) the following two expression vectors (g-1) and (g-2):
   (g-1) an expression vector comprising a nucleic acid encoding a cell surface molecule specifically recognizing human mesothelin; and
   (g-2) an expression vector comprising a nucleic acid encoding IL-7, and a nucleic acid encoding CCL19;
(h) the following two expression vectors (h-1) and (h-2):
   (h-1) an expression vector comprising a nucleic acid encoding a cell surface molecule specifically recognizing human mesothelin, and a nucleic acid encoding IL-7; and
   (h-2) an expression vector comprising a nucleic acid encoding CCL19;
(i) the following two expression vectors (i-1) and (i-2):
   (i-1) an expression vector comprising a nucleic acid encoding IL-7; and
   (i-2) an expression vector comprising a nucleic acid encoding a cell surface molecule specifically recognizing human mesothelin, and a nucleic acid encoding CCL19;
(j) the following two expression vectors (j-1) and (j-2):
   (j-1) an expression vector comprising a nucleic acid encoding a cell surface molecule specifically recognizing human mesothelin, and a nucleic acid encoding IL-7; and
   (j-2) an expression vector comprising a nucleic acid encoding a cell surface molecule specifically recognizing human mesothelin, and a nucleic acid encoding CCL19;
(k) the following two expression vectors (k-1) and (k-2):
   (k-1) an expression vector comprising a nucleic acid encoding a cell surface molecule specifically recognizing human mesothelin, and a nucleic acid encoding IL-7; and
   (k-2) an expression vector comprising a nucleic acid encoding IL-7, and a nucleic acid encoding CCL19;
(l) the following two expression vectors (l-1) and (l-2):
   (l-1) an expression vector comprising a nucleic acid encoding a cell surface molecule specifically recognizing human mesothelin, and a nucleic acid encoding CCL19; and
   (l-2) an expression vector comprising a nucleic acid encoding IL-7, and a nucleic acid encoding CCL19; and
(m) the following three expression vectors (m-1), (m-2) and (m-3):
   (m-1) an expression vector comprising a nucleic acid encoding a cell surface molecule specifically recognizing human mesothelin;
   (m-2) an expression vector comprising a nucleic acid encoding IL-7; and
   (m-3) an expression vector comprising a nucleic acid encoding CCL19.

The IL-7/CCL19 expression-anti-human mesothelin vector may further contain a nucleic acid encoding an additional immune function control factor such as IL-15, CCL21, IL-2, IL-4, IL-12, IL-13, IL-17, IL-18, IP-10, interferon-γ, MIP-1alpha, GM-CSF, M-CSF, TGF-β, TNF-α, or a checkpoint inhibiting antibody or its fragment. The nucleic acid encoding an additional immune function control factor is preferably a nucleic acid encoding an immune function control factor other than IL-12.

### (Nucleic Acid)

In the present specification, the “nucleic acid” can be any molecule of polymerized nucleotides and/or molecules having functions equivalent to those of the nucleotides. Examples thereof can include RNA which is a polymer of ribonucleotides, DNA which is a polymer of deoxyribonucleotides, a mixed polymer of ribonucleotides and deoxyribonucleotides, and a nucleotide polymer comprising a nucleotide analog. Alternatively, a nucleotide polymer comprising a nucleic acid derivative may be used. The nucleic acid may be a single-stranded nucleic acid or a double-stranded nucleic acid. The double-stranded nucleic acid also includes a double-stranded nucleic acid in which one of the strands hybridizes under stringent conditions to the other strand.

The nucleotide analog can be any molecule as long as the molecule is a ribonucleotide, a deoxyribonucleotide, RNA or DNA modified in order to improve or stabilize nuclease resistance, in order to enhance affinity for a complementary strand nucleic acid, in order to enhance cell permeability, or in order to visualize the molecule, as compared with RNA or DNA. The nucleotide analog may be a naturally occurring molecule or a non-natural molecule. Examples thereof include a nucleotide analog with a modified sugar moiety and a nucleotide analog with a modified phosphodiester bond.

The nucleotide analog with a modified sugar moiety can be any molecule as long as an arbitrary chemical structural substance is added to or replaced for a portion or the whole of the chemical structure of a sugar in a nucleotide. Specific examples thereof include a nucleotide analog substituted by 2′-O-methyl ribose, a nucleotide analog substituted by 2′-O-propyl ribose, a nucleotide analog substituted by 2′-methoxyethoxy ribose, a nucleotide analog substituted by 2′-O-methoxyethyl ribose, a nucleotide analog substituted by 2′-O-[2-(guanidium)ethyl]ribose, a nucleotide analog substituted by 2′-fluoro ribose, bridged nucleic acid (BNA) having two cyclic structures by the introduction of a bridged structure to the sugar moiety, more specifically, locked nucleic acid (LNA) with an oxygen atom at position 2′ and a carbon atom at position 4′ bridged via methylene, and ethylene bridged nucleic acid (ENA) [Nucleic Acid Research, 32, e175 (2004)], and can further include peptide nucleic acid (PNA) [Acc. Chem. Res., 32, 624 (1999)], oxypeptide nucleic acid (OPNA) [J. Am. Chem. Soc., 123, 4653 (2001)], and peptide ribonucleic acid (PRNA) [J. Am. Chem. Soc., 122, 6900 (2000)].

The nucleotide analog with a modified phosphodiester bond can be any molecule as long as an arbitrary chemical structural substance is added to or replaced for a portion or the whole of the chemical structure of a phosphodiester bond in a nucleotide. Specific examples thereof can include a nucleotide analog substituted by a phosphorothioate bond, and a nucleotide analog substituted by a N3′-P5′ phosphoramidate bond [Cell Engineering, 16, 1463-1473 (1997)][RNAi Method and Antisense Method, Kodansha Ltd. (2005)].

The nucleic acid derivative can be any molecule as long as the molecule is a nucleic acid with another chemical substance added thereto in order to improve or stabilize nuclease resistance, in order to enhance affinity for a complementary strand nucleic acid, in order to enhance cell permeability, or in order to visualize the molecule, as compared with a nucleic acid. Specific examples thereof can include a 5′-polyamine-added derivative, a cholesterol-added derivative, a steroid-added derivative, a bile acid-added derivative, a vitamin-added derivative, a Cy5-added derivative, a Cy3-added derivative, a 6-FAM-added derivative, and a biotin-added derivative.

### (Nucleic Acids Encoding Cell Surface Molecule Specifically Recognizing Human Mesothelin, IL-7, CCL19, Etc.)

Examples of the nucleic acid encoding a cell surface molecule specifically recognizing human mesothelin, the nucleic acid encoding IL-7, and the nucleic acid encoding CCL19 can include respective nucleic acids derived from a mammal and can preferably include respective nucleic acids derived from a human. Each of the nucleic acids can be appropriately selected according to the type of the cell to which the expression vector according to certain aspect(s) of the present invention is to be introduced. Sequence information on each of the nucleic acids can be appropriately obtained by the search of a publicly known document or a database such as NCBI (http://www.ncbi.nlm.nih.gov/guide/).

Examples of the nucleic acid encoding a cell surface molecule specifically recognizing human mesothelin can include a nucleic acid encoding CAR specifically recognizing human mesothelin.

Specific examples of the nucleic acid encoding single chain antibody comprised in the CAR specifically recognizing human mesothelin can include the following nucleic acids (1-1D) to (3-1D):
(1-1D) a nucleic acid encoding single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 30, light chain CDR2 consisting of the amino acid sequence shown by SEQ ID NO: 31, and light chain CDR3 consisting of the amino acid sequence shown by SEQ ID NO: 32;
(2-1D) a nucleic acid encoding single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 33, light chain CDR2 consisting of the amino acid sequence shown by SEQ ID NO: 31, and light chain CDR3 consisting of the amino acid sequence shown by SEQ ID NO: 32; and
(3-1D) a nucleic acid encoding single chain antibody comprising a heavy chain variable region comprising heavy chain CDR1 consisting of the amino acid sequence shown by SEQ ID NO: 34, heavy chain CDR2 consisting of the amino acid sequence shown by SEQ ID NO: 35, and heavy chain CDR3 consisting of the amino acid sequence shown by SEQ ID NO: 36, and a light chain variable region comprising light chain CDR1 consisting of the amino acid sequence shown by SEQ ID NO: 37, light chain CDR2 consisting of the amino acid sequence shown by SEQ ID NO: 38, and light chain CDR3 consisting of the amino acid sequence shown by SEQ ID NO: 39.

Specific examples of additional form 1 of the nucleic acid encoding single chain antibody comprised in the CAR specifically recognizing human mesothelin can include the following nucleic acids (1-2D) to (5-2D):
(1-2D) a nucleic acid encoding single chain antibody comprising a heavy chain variable region consisting of an amino acid sequence having 85% or higher, preferably 90% or higher, more preferably 95% or higher, further preferably 98% or higher sequence identity to the amino acid sequence shown by SEQ ID NO: 1, and a light chain variable region consisting of an amino acid sequence having 85% or higher, preferably 90% or higher, more preferably 95% or higher, further preferably 98% or higher sequence identity to the amino acid sequence shown by SEQ ID NO: 2;
(2-2D) a nucleic acid encoding single chain antibody comprising a heavy chain variable region consisting of an amino acid sequence having 85% or higher, preferably 90% or higher, more preferably 95% or higher, further preferably 98% or higher sequence identity to the amino acid sequence shown by SEQ ID NO: 17, and a light chain variable region consisting of an amino acid sequence having 85% or higher, preferably 90% or higher, more preferably 95% or higher, further preferably 98% or higher sequence identity to the amino acid sequence shown by SEQ ID NO: 18;
(3-2D) a nucleic acid encoding single chain antibody comprising a heavy chain variable region consisting of an amino acid sequence having 85% or higher, preferably 90% or higher, more preferably 95% or higher, further preferably 98% or higher sequence identity to the amino acid sequence shown by SEQ ID NO: 19, and a light chain variable region consisting of an amino acid sequence having 85% or higher, preferably 90% or higher, more preferably 95% or higher, further preferably 98% or higher sequence identity to the amino acid sequence shown by SEQ ID NO: 20;
(4-2D) a nucleic acid encoding single chain antibody comprising a heavy chain variable region consisting of an amino acid sequence having 85% or higher, preferably 90% or higher, more preferably 95% or higher, further preferably 98% or higher sequence identity to the amino acid sequence shown by SEQ ID NO: 1, and a light chain variable region consisting of an amino acid sequence having 85% or higher, preferably 90% or higher, more preferably 95% or higher, further preferably 98% or higher sequence identity to the amino acid sequence shown by SEQ ID NO: 18; and
(5-2D) a nucleic acid encoding single chain antibody comprising a heavy chain variable region consisting of an amino acid sequence having 85% or higher, preferably 90% or higher, more preferably 95% or higher, further preferably 98% or higher sequence identity to the amino acid sequence shown by SEQ ID NO: 17, and a light chain variable region consisting of an amino acid sequence having 85% or higher, preferably 90% or higher, more preferably 95% or higher, further preferably 98% or higher sequence identity to the amino acid sequence shown by SEQ ID NO: 2.

Specific examples of additional form 2 of the nucleic acid encoding single chain antibody comprised in the CAR specifically recognizing human mesothelin can include the following nucleic acids (1-3D) to (5-3D):
(1-3D) a nucleic acid encoding single chain antibody comprising a heavy chain variable region consisting of the amino acid sequence shown by SEQ ID NO: 1, and a light chain variable region consisting of the amino acid sequence shown by SEQ ID NO: 2;
(2-3D) a nucleic acid encoding single chain antibody comprising a heavy chain variable region consisting of the amino acid sequence shown by SEQ ID NO: 17, and a light chain variable region consisting of the amino acid sequence shown by SEQ ID NO: 18;
(3-3D) a nucleic acid encoding single chain antibody comprising a heavy chain variable region consisting of the amino acid sequence shown by SEQ ID NO: 19, and a light chain variable region consisting of the amino acid sequence shown by SEQ ID NO: 20;
(4-3D) a nucleic acid encoding single chain antibody comprising a heavy chain variable region consisting of the amino acid sequence shown by SEQ ID NO: 1, and a light chain variable region consisting of the amino acid sequence shown by SEQ ID NO: 18; and
(5-3D) a nucleic acid encoding single chain antibody comprising a heavy chain variable region consisting of the amino acid sequence shown by SEQ ID NO: 17, and a light chain variable region consisting of the amino acid sequence shown by SEQ ID NO: 2.

Specific examples of additional form 3 of the nucleic acid encoding single chain antibody comprised in the CAR specifically recognizing human mesothelin can include the following nucleic acids (1-4D) to (9-4D):
(1-4D) a nucleic acid encoding single chain antibody sequentially comprising a heavy chain variable region consisting of the amino acid sequence shown by SEQ ID NO: 1, a peptide linker consisting of the amino acid sequence shown by SEQ ID NO: 40, and a light chain variable region consisting of the amino acid sequence shown by SEQ ID NO: 2;
(2-4D) a nucleic acid encoding single chain antibody sequentially comprising a heavy chain variable region consisting of the amino acid sequence shown by SEQ ID NO: 17, a peptide linker consisting of the amino acid sequence shown by SEQ ID NO: 40, and a light chain variable region consisting of the amino acid sequence shown by SEQ ID NO: 18;
(3-4D) a nucleic acid encoding single chain antibody sequentially comprising a light chain variable region consisting of the amino acid sequence shown by SEQ ID NO: 2, a peptide linker consisting of the amino acid sequence shown by SEQ ID NO: 40, and a heavy chain variable region consisting of the amino acid sequence shown by SEQ ID NO: 1;
(4-4D) a nucleic acid encoding single chain antibody sequentially comprising a heavy chain variable region consisting of the amino acid sequence shown by SEQ ID NO: 1, a peptide linker consisting of the amino acid sequence shown by SEQ ID NO: 41, and a light chain variable region consisting of the amino acid sequence shown by SEQ ID NO: 2;
(5-4D) a nucleic acid encoding single chain antibody sequentially comprising a light chain variable region consisting of the amino acid sequence shown by SEQ ID NO: 2, a peptide linker consisting of the amino acid sequence shown by SEQ ID NO: 41, and a heavy chain variable region consisting of the amino acid sequence shown by SEQ ID NO: 1;
(6-4D) a nucleic acid encoding single chain antibody sequentially comprising a heavy chain variable region consisting of the amino acid sequence shown by SEQ ID NO: 19, a peptide linker consisting of the amino acid sequence shown by SEQ ID NO: 40, and a light chain variable region consisting of the amino acid sequence shown by SEQ ID NO: 20;
(7-4D) a nucleic acid encoding single chain antibody sequentially comprising a light chain variable region consisting of the amino acid sequence shown by SEQ ID NO: 20, a peptide linker consisting of the amino acid sequence shown by SEQ ID NO: 40, and a heavy chain variable region consisting of the amino acid sequence shown by SEQ ID NO: 19;
(8-4D) a nucleic acid encoding single chain antibody sequentially comprising a heavy chain variable region consisting of the amino acid sequence shown by SEQ ID NO: 19, a peptide linker consisting of the amino acid sequence shown by SEQ ID NO: 41, and a light chain variable region consisting of the amino acid sequence shown by SEQ ID NO: 20; and
(9-4D) a nucleic acid encoding single chain antibody sequentially comprising a light chain variable region consisting of the amino acid sequence shown by SEQ ID NO: 20, a peptide linker consisting of the amino acid sequence shown by SEQ ID NO: 41, and a heavy chain variable region consisting of the amino acid sequence shown by SEQ ID NO: 19.

Examples of the nucleic acid encoding a polypeptide of a transmembrane region comprised in the CAR can include a nucleic acid encoding a polypeptide of a human CD8 transmembrane region comprising an amino acid sequence having 85% or higher, preferably 90% or higher, more preferably 95% or higher, further preferably 98% or higher sequence identity to the amino acid sequence shown by SEQ ID NO: 21. Examples of the nucleic acids encoding polypeptides of CD28, 4-1BB, and CD3ζ intracellular regions in an immunocompetent cell activating signaling region comprised in the CAR can include a nucleic acid encoding a polypeptide that comprises an amino acid sequence having 85% or higher, preferably 90% or higher, more preferably 95% or higher, further preferably 98% or higher sequence identity to the amino acid sequence of the human CD28 intracellular region shown by SEQ ID NO: 22, and has action equivalent to that of the amino acid sequence shown by SEQ ID NO: 22, a nucleic acid encoding a polypeptide that comprises an amino acid sequence having 85% or higher, preferably 90% or higher, more preferably 95% or higher, further preferably 98% or higher sequence identity to the amino acid sequence of the human 4-1BB intracellular region shown by SEQ ID NO: 23, and has action equivalent to that of the amino acid sequence shown by SEQ ID NO: 23, and a nucleic acid encoding a polypeptide that comprises an amino acid sequence having 85% or higher, preferably 90% or higher, more preferably 95% or higher, further preferably 98% or higher sequence identity to the amino acid sequence of the human CD3ζ intracellular region shown by SEQ ID NO: 24, and has action equivalent to that of the amino acid sequence shown by SEQ ID NO: 24, and a combination thereof, preferably a nucleic acid encoding a polypeptide of the human CD28 intracellular region shown in SEQ ID NO: 22, a nucleic acid encoding a polypeptide of the human 4-1BB intracellular region shown in SEQ ID NO: 23, and a nucleic acid encoding a polypeptide of the human CD3ζ intracellular region shown in SEQ ID NO: 24 in order from the upstream side (5′-terminal side).

Examples of the nucleic acid encoding IL-7 can include a nucleic acid encoding a polypeptide that comprises an amino acid sequence having 85% or higher, preferably 90% or higher, more preferably 95% or higher, further preferably 98% or higher sequence identity to the amino acid sequence shown by SEQ ID NO: 42, and has action equivalent to that of the amino acid sequence shown by SEQ ID NO: 42, specifically, a nucleic acid consisting of the nucleotide sequence shown by SEQ ID NO: 44. The nucleic acid encoding IL-7 may be a nucleic acid having 80% or higher, preferably 85% or higher, more preferably 90% or higher, further preferably 95% or higher, most preferably 98% or higher sequence identity to the nucleic acid consisting of the nucleotide sequence shown by SEQ ID NO: 44 as long as the nucleic acid has action of enhancing a cell proliferation rate or a cell survival rate by IL-7. Examples of the nucleic acid encoding CCL19 can include a nucleic acid encoding a polypeptide that comprises an amino acid sequence having 85% or higher, preferably 90% or higher, more preferably 95% or higher, further preferably 98% or higher sequence identity to the amino acid sequence shown by SEQ ID NO: 43, and has action equivalent to that of the amino acid sequence shown by SEQ ID NO: 43, specifically, a nucleic acid consisting of the nucleotide sequence shown by SEQ ID NO: 45. A nucleic acid having 80% or higher, preferably 85% or higher, more preferably 90% or higher, further preferably 95% or higher, most preferably 98% or higher sequence identity to the nucleic acid consisting of the nucleotide sequence shown by SEQ ID NO: 45 may be used as long as the nucleic acid has the cell migrating action of CCL19.

### (Suicide Gene)

The expression vector according to certain aspect(s) of the present invention may comprise a nucleic acid encoding a suicide gene. The suicide gene means a gene that directly or secondarily induces a substance having cellular toxicity through its expression and has the function of killing its own cell. Owing to the expression vector according to certain aspect(s) of the present invention comprising the nucleic acid encoding the suicide gene, for example, an immunocompetent cell present in a living body can be controlled by the administration of a drug activating the function of the suicide gene after disappearance of tumor according to the course of treatment of cancer. IL-7 or CCL19, unlike other cytokines, is less likely to cause cytokine release syndrome or tumorigenic transformation of transfected cells as adverse reactions. However, the enhanced functions of the immunocompetent cell harboring the expression vector according to certain aspect(s) of the present invention may cause unexpected influence of cytokines, etc. released upon attack on a target cancer tissue on neighboring tissues. In such a case, the nucleic acid encoding the suicide gene, comprised in the expression vector according to certain aspect(s) of the present invention is capable of reliably reducing a risk of cytokine release syndrome.

Examples of the suicide gene can include a gene encoding herpes simplex virus thymidine kinase (HSV-TK) or inducible caspase 9 described in a document given below. Examples of the drug activating the function of such a gene can include ganciclovir for the former and AP1903 which is chemical induction of dimerization (CID) for the latter.

### (Obtainment of Nucleic Acid Sequence Information and Production of Nucleic Acid)

Each of the nucleic acids comprised in the expression vector according to certain aspect(s) of the present invention may be a naturally derived nucleic acid or an artificially synthesized nucleic acid, and can be appropriately selected according to the type of the cell to which the expression vector according to certain aspect(s) of the present invention is to be introduced. Their sequence information can be appropriately obtained by the search of a publicly known document or a database such as NCBI (http://www.ncbi.nlm.nih.gov/guide/).

Each of the nucleic acids can be produced by a publicly known technique such as a chemical synthesis method or a PCR amplification method on the basis of information on the nucleotide sequence of each of the nucleic acids. Codons selected for encoding amino acids may be engineered in order to optimize nucleic acid expression in host cells of interest.

### (Arrangement of Each Nucleic Acid)

When the expression vector according to certain aspect(s) of the present invention is the expression vector (f) comprising a nucleic acid encoding a cell surface molecule specifically recognizing human mesothelin, a nucleic acid encoding IL-7, and a nucleic acid encoding CCL19, any of the nucleic acids may be arranged upstream or downstream of any of the nucleic acids. Specifically, in the case of containing, for example, a nucleic acid encoding anti-human mesothelin CAR as the nucleic acid encoding a cell surface molecule specifically recognizing human mesothelin, the arrangement may be the nucleic acid encoding anti-human mesothelin CAR, the nucleic acid encoding IL-7 and the nucleic acid encoding CCL19, may be the nucleic acid encoding anti-human mesothelin CAR, the nucleic acid encoding CCL19 and the nucleic acid encoding IL-7, may be the nucleic acid encoding IL-7, the nucleic acid encoding CCL19 and the nucleic acid encoding anti-human mesothelin CAR, may be the nucleic acid encoding IL-7, the nucleic acid encoding anti-mesothelin CAR and the nucleic acid encoding CCL19, may be the nucleic acid encoding CCL19, the nucleic acid encoding anti-mesothelin CAR, and the nucleic acid encoding IL-7, or may be the nucleic acid encoding CCL19, the nucleic acid encoding IL-7 and the nucleic acid encoding anti-human mesothelin CAR, in order from the upstream side (5′-terminal side).

In the expression vector (g-2), (k-2), or (l-2) comprising a nucleic acid encoding IL-7, and a nucleic acid encoding CCL19 in the vector according to certain aspect(s) of the present invention, the arrangement of the nucleic acid encoding IL-7 and the nucleic acid encoding CCL19 is not particularly limited. The nucleic acid encoding CCL19 may be arranged upstream or downstream of the nucleic acid encoding IL-7.

In the expression vector (h-1), (j-1) or (k-1) comprising a nucleic acid encoding a cell surface molecule specifically recognizing human mesothelin, and a nucleic acid encoding IL-7 in the vector according to certain aspect(s) of the present invention, the arrangement of the nucleic acid encoding a cell surface molecule specifically recognizing human mesothelin and the nucleic acid encoding IL-7 are not particularly limited. The nucleic acid encoding IL-7 may be arranged upstream or downstream of the nucleic acid encoding a cell surface molecule specifically recognizing human mesothelin.

In the expression vector (i-2), (j-2) or (l-1) comprising a nucleic acid encoding a cell surface molecule specifically recognizing human mesothelin, and a nucleic acid encoding CCL19 in the vector according to certain aspect(s) of the present invention, the arrangement of the nucleic acid encoding a cell surface molecule specifically recognizing human mesothelin and the nucleic acid encoding CCL19 are not particularly limited. The nucleic acid encoding CCL19 may be arranged upstream or downstream of the nucleic acid encoding a cell surface molecule specifically recognizing human mesothelin.

### (Transcription)

The nucleic acid encoding a cell surface molecule specifically recognizing mesothelin, the nucleic acid encoding IL-7, the nucleic acid encoding CCL19, and the nucleic acid encoding a suicide gene may be transcribed under different promoters or may be transcribed under one promoter using an internal ribosome entry site (IRES) or self-cleaving 2A peptide.

An arbitrary nucleic acid may be comprised between the nucleic acid encoding IL-7 and the nucleic acid encoding CCL19 in the case of transcribing these nucleic acids under one promoter using an internal ribosome entry site (IRES) or self-cleaving 2A peptide, or between the nucleic acid encoding a cell surface molecule specifically recognizing mesothelin and the nucleic acid encoding IL-7 and the nucleic acid encoding CCL19 in the case of comprising the nucleic acid encoding a cell surface molecule specifically recognizing mesothelin, as long as each of the nucleic acids can be expressed. The linking is preferably achieved via a sequence encoding self-cleaving peptide (2A peptide) or IRES, preferably a sequence encoding 2A peptide. The linking using such a sequence permits efficient expression of each of the nucleic acids.

The 2A peptide is a virus-derived self-cleaving peptide and is characterized in that the amino acid sequence shown by SEQ ID NO: 46 is cleaved at G-P (position of one residue from the C terminus) in the endoplasmic reticulum (Szymczak et al., Expert Opin. Biol. Ther. 5 (5): 627-638 (2005)). Hence, nucleic acids flanking the 2A peptide are each expressed independently in a cell via the 2A peptide.

The 2A peptide is preferably 2A peptide derived from picornavirus, rotavirus, insect virus, Aphthovirus or Trypanosoma virus, more preferably picornavirus-derived 2A peptide (F2A) shown in SEQ ID NO: 47.

### (Type of Vector)

The type of the vector for the expression vector according to certain aspect(s) of the present invention may be a linear form or a circular form and may be a non-viral vector such as a plasmid, may be a viral vector, or may be a vector based on a transposon. Such a vector may contain a control sequence such as a promoter or a terminator, or a selective marker sequence such as a drug resistance gene or a reporter gene. The nucleic acid encoding IL-7 and the nucleic acid encoding CCL19 are operably arranged downstream of the promoter sequence so that each of the nucleic acids can be efficiently transcribed.

Examples of the promoter can include: a virus-derived promoter such as retrovirus LTR promoter, SV40 early promoter, cytomegalovirus promoter, and herpes simplex virus thymidine kinase promoter; and a mammal-derived promoter such as phosphoglycerate kinase (PGK) promoter, Xist promoter, R-actin promoter, and RNA polymerase II promoter. Alternatively, tetracycline-responsive promoter which is induced by tetracycline, Mx1 promoter which is induced by interferon, or the like may be used. Use of the promoter which is induced by a particular substance in the expression vector according to certain aspect(s) of the present invention permits control of induction of IL-7 and CCL19 expression according to the course of treatment of cancer, for example, when the immunocompetent cell containing the vector according to certain aspect(s) of the present invention is used as a pharmaceutical composition for use in the treatment of cancer.

Examples of the viral vector can include a retrovirus vector, a lentivirus vector, an adenovirus vector, and an adeno-associated virus vector and can preferably include a retrovirus vector, more preferably a pMSGV vector (Tamada k et al., Clin Cancer Res 18: 6436-6445 (2002)) and a pMSCV vector (manufactured by Takara Bio Inc.). Use of a retrovirus vector permits long-term and stable expression of a transgene because the transgene is integrated in the genome of a host cell.

In order to confirm the containment of the expression vector according to certain aspect(s) of the present invention in the immunocompetent cell, for example, the expression of CAR can be examined by flow cytometry, Northern blotting, Southern blotting, PCR such as RT-PCR, ELISA, or Western blotting when the expression vector contains a nucleic acid encoding CAR, and the expression of a marker gene inserted in the expression vector according to certain aspect(s) of the present invention can be examined when the expression vector contains the marker gene.

### (Pharmaceutical Composition)

The pharmaceutical composition according to certain aspect(s) of the present invention is not limited as long as the pharmaceutical composition comprises the immunocompetent cell according to certain aspect(s) of the present invention and a pharmaceutically acceptable additive. Examples of the additive can include saline, buffered saline, a cell culture medium, dextrose, injectable water, glycerol, ethanol, and a combination thereof, a stabilizer, a solubilizer and a surfactant, a buffer and an antiseptic, a tonicity agent, a filler, and a lubricant. Since the immunocompetent cell in the pharmaceutical composition according to certain aspect(s) of the present invention has a signaling region that induces the activation of the immunocompetent cell, the pharmaceutical composition according to certain aspect(s) of the present invention may serve as a pharmaceutical composition for use in the treatment of cancer. Such a pharmaceutical composition for use in the treatment of cancer may contain a package insert, a label, a package, or the like stating a use method, etc. for use in the treatment of cancer. Since the immunocompetent cell in the pharmaceutical composition according to certain aspect(s) of the present invention has suppressive effects on tumor recurrence, the pharmaceutical composition according to certain aspect(s) of the present invention may serve as a pharmaceutical composition for use in the suppression of tumor recurrence. Such a pharmaceutical composition for use in the suppression of tumor recurrence may contain a package insert, a label, a package, or the like stating a use method, etc. for use in the suppression of tumor recurrence.

The pharmaceutical composition according to certain aspect(s) of the present invention can be administered to a test subject in need thereof by use of a method known to those skilled in the art. Examples of the administration method can include intravenous, intratumoral, intracutaneous, subcutaneous, intramuscular, intraperitoneal, intraarterial, intramedullary, intracardiac, intraarticular, intrasynovial, intracranial, intrathecal, and subarachnoidal (spinal fluid) injection.

In an exemplary method, the pharmaceutical composition according to certain aspect(s) of the present invention can be independently administered in one portion or several divided portions 4 times, 3 times, twice, or once a day, at a 1-day, 2-day, 3-day, 4-day, or 5-day interval, once a week, at a 7-day, 8-day, or 9-day interval, twice a week, once a month, or twice a month.

The cancer for the pharmaceutical composition according to certain aspect(s) of the present invention is not particularly limited and is preferably a cancer type expressing mesothelin in a cancer tissue, or a cancer type derived from cancer cells expressing mesothelin. Examples thereof can include: cancer such as mesothelioma, colorectal cancer (colon cancer or rectum cancer), pancreatic cancer, thymic cancer, bile duct cancer, lung cancer (adenocarcinoma, squamous cell cancer, adenosquamous cancer, undifferentiated cancer, large-cell cancer, and small-cell cancer), skin cancer, breast cancer, prostate cancer, urinary bladder cancer, vaginal cancer, neck cancer, uterine cancer, liver cancer, kidney cancer, pancreatic cancer, spleen cancer, tracheal cancer, bronchial cancer, colon cancer, small intestine cancer, stomach cancer, esophageal cancer, gallbladder cancer, testis cancer, and ovary cancer; cancer of a bone tissue, a cartilage tissue, a fat tissue, a muscle tissue, a vascular tissue, and a hematopoietic tissue; sarcoma such as chondrosarcoma, Ewing's sarcoma, malignant hemangioendothelioma, malignant schwannoma, osteosarcoma, and soft tissue sarcoma; blastoma such as hepatoblastoma, medulloblastoma, nephroblastoma, neuroblastoma, pancreatoblastoma, pleuropulmonary blastoma, and retinoblastoma; and embryonic cell tumor. The dose of the pharmaceutical composition to be administered can be a therapeutically effective amount. Examples thereof can preferably include 1×10⁴ to 1×10¹⁰ cells, preferably 1×10⁵ to 1×10⁹ cells, more preferably 5×10⁶ to 5×10⁸ cells, in terms of the number of cells to be administered in a single dose.

The pharmaceutical composition according to certain aspect(s) of the present invention can be used in combination with an additional anticancer agent. Examples of the additional anticancer agent can include: an alkylating agent such as cyclophosphamide, bendamustine, ifosfamide, and dacarbazine; an antimetabolite such as pentostatin, fludarabine, cladribine, methotrexate, 5-fluorouracil, 6-mercaptopurine, and enocitabine; a molecular targeting drug such as rituximab, cetuximab, and trastuzumab; a kinase inhibitor such as imatinib, gefitinib, erlotinib, afatinib, dasatinib, sunitinib, and trametinib; a proteasome inhibitor such as bortezomib; a calcineurin inhibitory drug such as cyclosporine and tacrolimus; an anticancer antibiotic such as idarubicin, doxorubicin, and mitomycin C; a vegetable alkaloid such as irinotecan and etoposide; a platinum-containing drug such as cisplatin, oxaliplatin, and carboplatin; a hormone therapeutic such as tamoxifen and bicalutamide; and an immunoregulatory drug such as interferon, nivolumab, and pembrolizumab.

Examples of the method for “using the pharmaceutical composition according to certain aspect(s) of the present invention in combination with the additional anticancer agent” can include a method of using the additional anticancer agent in a process and then using the pharmaceutical composition according to certain aspect(s) of the present invention, a method of concurrently using the pharmaceutical composition according to certain aspect(s) of the present invention and the additional anticancer agent, and a method of using the pharmaceutical composition according to certain aspect(s) of the present invention in a process and then using the additional anticancer agent. Combined use of the pharmaceutical composition according to certain aspect(s) of the present invention for use in the treatment of cancer with the additional anticancer agent further improves therapeutic effects on cancer and can reduce the adverse reactions of each anticancer agent by decreasing the administration frequency or dose of the anticancer agent. Alternatively, the additional anticancer agent may be contained in the pharmaceutical composition according to certain aspect(s) of the present invention.

### (Other Aspects of Present Invention)

Examples of additional aspect 1 of the present invention can include 1) a method for treating cancer, comprising administering the immunocompetent cell according to certain aspect(s) of the present invention to a patient in need of treatment of cancer, 2) the immunocompetent cell according to certain aspect(s) of the present invention for use as a pharmaceutical composition, and 3) use of the immunocompetent cell according to certain aspect(s) of the present invention in the preparation of a pharmaceutical composition.

Examples of additional aspect 2 of the present invention can include chimeric antigen receptor (CAR) having any of single chain antibodies given below, a transmembrane region, and a signaling region that induces the activation of the immunocompetent cell. Such CAR, when expressed in an immunocompetent cell, is capable of activating the immunocompetent cell through stimulation with human mesothelin.
(1-1) single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 30, light chain CDR2 consisting of the amino acid sequence shown by SEQ ID NO: 31, and light chain CDR3 consisting of the amino acid sequence shown by SEQ ID NO: 32;
(2-1) single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 33, light chain CDR2 consisting of the amino acid sequence shown by SEQ ID NO: 31, and light chain CDR3 consisting of the amino acid sequence shown by SEQ ID NO: 32; and
(3-1) single chain antibody comprising a heavy chain variable region comprising heavy chain CDR1 consisting of the amino acid sequence shown by SEQ ID NO: 34, heavy chain CDR2 consisting of the amino acid sequence shown by SEQ ID NO: 35, and heavy chain CDR3 consisting of the amino acid sequence shown by SEQ ID NO: 36, and a light chain variable region comprising light chain CDR1 consisting of the amino acid sequence shown by SEQ ID NO: 37, light chain CDR2 consisting of the amino acid sequence shown by SEQ ID NO: 38, and light chain CDR3 consisting of the amino acid sequence shown by SEQ ID NO: 39.

Examples of additional aspect 3 of the present invention can include a kit for producing an immunocompetent cell having the expression vector according to certain aspect(s) of the present invention. Such a kit is not particularly limited as long as the kit has the expression vector according to certain aspect(s) of the present invention. The kit may comprise an instruction for producing the immunocompetent cell according to certain aspect(s) of the present invention, and a reagent for use in introducing the expression vector according to certain aspect(s) of the present invention to an immunocompetent cell.

Examples of additional aspect 4 of the present invention can include a method for suppressing the recurrence of cancer, comprising administering an immunocompetent cell that expresses a cell surface molecule (preferably CAR having single chain antibody specifically recognizing human mesothelin), IL-7 and CCL19 at the same time to a subject.

Hereinafter, the present invention will be described more specifically with reference to Examples. However, the technical scope of the present invention is not limited by these examples.

### [Example 7] Production of Anti-Human Mesothelin CAR (Synthesis of scFv Sequence and DNA Fragment of Anti-Human Mesothelin CAR)

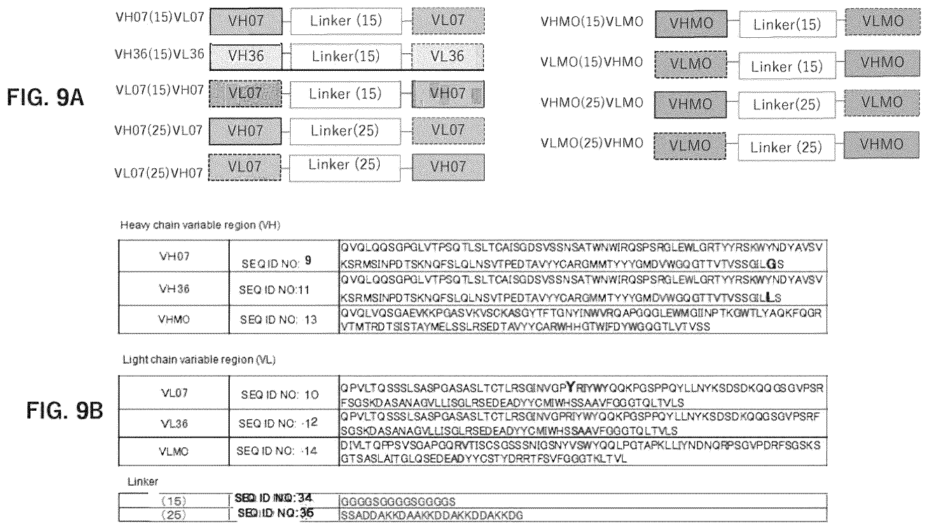

The sequences of 9 types of anti-human mesothelin scFvs shown in FIGS. 9A-9B were designed in order to compare thereamong the sequences and order of VL and VH, and the type of a suitable signal peptide.

VH07(15)VL07 consists of the amino acid sequence of a heavy chain variable region shown by SEQ ID NO: 1, the amino acid sequence of a peptide linker shown by SEQ ID NO: 40, and the amino acid sequence of a light chain variable region shown by SEQ ID NO: 2.

VH36(15)VL36 consists of the amino acid sequence of a heavy chain variable region shown by SEQ ID NO: 17, the amino acid sequence of a peptide linker shown by SEQ ID NO: 40, and the amino acid sequence of a light chain variable region shown by SEQ ID NO: 18.

VL07(15)VH07 consists of the amino acid sequence of a light chain variable region shown by SEQ ID NO: 2, the amino acid sequence of a peptide linker shown by SEQ ID NO: 40, and the amino acid sequence of a heavy chain variable region shown by SEQ ID NO: 1.

VH07(25)VL07 consists of the amino acid sequence of a heavy chain variable region shown by SEQ ID NO: 1, the amino acid sequence of a peptide linker shown by SEQ ID NO: 41, and the amino acid sequence of a light chain variable region shown by SEQ ID NO: 2.

VL07(25)VH07 consists of the amino acid sequence of a light chain variable region shown by SEQ ID NO: 2, the amino acid sequence of a peptide linker shown by SEQ ID NO: 41, and the amino acid sequence of a heavy chain variable region shown by SEQ ID NO: 1.

VHMO(15)VLMO consists of the amino acid sequence of a heavy chain variable region shown by SEQ ID NO: 19, the amino acid sequence of a peptide linker shown by SEQ ID NO: 40, and the amino acid sequence of a light chain variable region shown by SEQ ID NO: 20.

VLMO(15)VHMO consists of the amino acid sequence of a light chain variable region shown by SEQ ID NO: 20, the amino acid sequence of a peptide linker shown by SEQ ID NO: 40, and the amino acid sequence of a heavy chain variable region shown by SEQ ID NO: 19.

VHMO(25)VLMO consists of the amino acid sequence of a heavy chain variable region shown by SEQ ID NO: 19, the amino acid sequence of a peptide linker shown by SEQ ID NO: 41, and the amino acid sequence of a light chain variable region shown by SEQ ID NO: 20.

VLMO(25)VHMO consists of the amino acid sequence of a light chain variable region shown by SEQ ID NO: 20, the amino acid sequence of a peptide linker shown by SEQ ID NO: 41, and the amino acid sequence of a heavy chain variable region shown by SEQ ID NO: 19.

The amino acid sequences shown by SEQ ID NO: 15 and SEQ ID NO: 17 differ in that the 127th amino acid is glycine (G) in SEQ ID NO: 15 and is leucine (L) in the amino acid sequence shown by SEQ ID NO: 17. The amino acid sequences shown by SEQ ID NO: 16 and SEQ ID NO: 18 differ in that the 33rd amino acid in SEQ ID NO: 16 is tyrosine (Y), which is deleted in the amino acid sequence shown by SEQ ID NO: 18.

A DNA fragment encoding the amino acid sequence of each of the anti-human mesothelin scFvs was synthesized.

(Production of Anti-Human Mesothelin IL-7/CCL19 CAR Expression Vector for Expression of IL-7/CCL19 and HSV-TK, and Conv. Anti-Human Mesothelin CAR Expression Vector without Expression of IL-7/CCL19)

CAR-T cell therapy may cause systemic adverse reactions such as cytokine release syndrome due to strong immune response to a target antigen. A CAR construct harboring a herpes virus-derived thymidine kinase HSV-TK gene as a suicide gene was produced in order to cope with the problem. If T cells are transfected with the construct so that the CAR-expressing T cells express HSV-TK, the addition of a cytomegalovirus therapeutic drug ganciclovir induces the apoptosis of the CAR-T cells and kills these cells. Therefore, the CAR-T cells in the body are controllable by the administration of ganciclovir.

First, a third-generation CAR construct sequentially having anti-human mesothelin scFv, human CD8 transmembrane region and human CD28-4-1BB-CD3ζ intracellular signaling region from the N-terminal side was produced in accordance with the method described in Patent Document 2. 2A peptide F2A was added to the C terminus of the construct, and human IL-7-F2A-human CCL19-F2A-HSV-TK was further added to downstream thereof. The obtained construct sequentially having scFv, human CD8 transmembrane region, human CD28-4-1BB-CD3ζ intracellular signaling region, human IL-7, human CCL19, and HSV-TK was inserted to a pMSGV1 retrovirus expression vector (Tamada k et al., Clin Cancer Res 18: 6436-6445 (2012)) to produce a pMSGV vector for expression of anti-human mesothelin scFv, human CD8 transmembrane region, human CD28-4-1BB-CD3ζ intracellular signaling region, human IL-7, human CCL19, and HSV-TK. Next, the anti-human mesothelin scFv region in the pMSGV vector was replaced by restriction enzyme (NcoI and NotI) treatment and ligation with each anti-human mesothelin scFv DNA fragment synthesized by the method described in the section “Synthesis of scFv sequence and DNA fragment of anti-human mesothelin CAR” to produce each “IL-7/CCL19 expression-anti-human mesothelin CAR vector”. The pMSGV1 vector has immune globulin G-derived signal peptide T (SEQ ID NO: 25) on the N-terminal side of scFv. As for the vector having the VH07(15)VL07 DNA fragment replaced for the scFv region, the signal peptide T shown in SEQ ID NO: 25 was replaced with signal peptide P shown in SEQ ID NO: 26 as a signal peptide for production. In addition, a “Conv. anti-human mesothelin CAR vector” was produced as a control without IL-7 and CCL19 in the same way as the method described above except that HSV-TK was used instead of the human IL-7-F2A-human CCL19-F2A-HSV-TK.

(Production of Retrovirus Harboring IL-7/CCL19 Expression-Anti-Human Mesothelin CAR Vector or Conv. Anti-Human Mesothelin CAR Vector)

Retrovirus was produced for transfection of T cells. A GP2-293 packaging cell line (manufactured by Takara Bio Inc.) was transfected with each of the IL-7/CCL19 expression-anti-human mesothelin CAR vectors or the Conv. anti-human mesothelin CAR vector and a p-Ampho plasmid (manufactured by Takara Bio Inc.) using Lipofectamine 3000 (manufactured by Life Technologies Corp.) to produce retrovirus harboring the IL-7/CCL19 expression-anti-human mesothelin CAR vector or the Conv. anti-human mesothelin CAR vector. A supernatant containing the retrovirus was recovered 48 hours after the transfection.

The culture solution used for the GP2-293 cells was DMEM supplemented with 10% FCS and 1% penicillin-streptomycin (manufactured by Wako Pure Chemical Industries, Ltd.). The culture solution used for T cells for use in Examples mentioned later was GT-T551 containing 2.0% human AB blood type serum (manufactured by Sigma-Aldrich Co. LLC), 1% penicillin-streptomycin (manufactured by Wako Pure Chemical Industries, Ltd.), and 2.5 μg/ml amphotericin B (manufactured by Bristol-Myers Squibb Company).

### (Transduction of T Cell)

2×10⁶ peripheral blood mononuclear cells collected from the blood of a healthy donor were cultured with IL-2 (manufactured by PeproTech, Inc.) at 37° C. for 3 days in a 5% CO₂ incubator on a plate on which an anti-CD3 monoclonal antibody (5 μg/ml) and RetroNectin® (manufactured by Takara Bio Inc., 25 μg/ml) were immobilized for activation of T cells. On day 2 after the start of culture, the supernatant containing the produced retrovirus harboring the IL-7/CCL19 expression-anti-human mesothelin CAR vector or the Conv. anti-human mesothelin CAR vector was added at 500 μl/well to a surface-untreated 24-well plate coated in advance with 25 μg/ml RetroNectin (manufactured by Takara Bio Inc.), and centrifuged at 2000 g for 2 hours to produce a retrovirus preload plate. A total of two such plates were produced, washed with 1.5% BSA/PBS after the completion of centrifugation, and stored at 4° C. until use. On culture day 3, the activated cells were recovered from the plate and adjusted as a cell suspension (1×10⁵ cells/ml). This cell suspension was added at 1 ml/well to the retrovirus preload plate and cultured at 37° C. for 24 hours in the presence of IL-2 in a 5% CO₂ incubator for the first retrovirus infection. On the next day (culture day 4), the cell lysate in each well was transferred to the stored second virus preload plate, centrifuged at 500 g for 1 minute, and then cultured at 37° C. for 4 hours for the second infection. After the culture at 37° C. for 4 hours, 1 ml of the cell suspension in each well was transferred to a fresh 12-well cell culture plate, diluted 4-fold with a fresh culture solution (GT-T551) containing IL-2, and cultured at 37° C. in a 5% CO₂ incubator. The culture was continued up to 7 days counted from the start date of culture of the peripheral blood mononuclear cells to obtain “anti-human mesothelin CAR-IL-7/CCL19-expressing T cells” as T cells harboring the IL-7/CCL19 expression-anti-human mesothelin CAR vector or “anti-human mesothelin CAR-expressing T cells” as T cells harboring the Conv. anti-human mesothelin CAR vector. The anti-human mesothelin CAR-IL-7/CCL19-expressing T cells contained an exogenous nucleic acid encoding anti-human mesothelin CAR, an exogenous nucleic acid encoding IL-7, and an exogenous nucleic acid encoding CCL19. The anti-human mesothelin CAR-expressing T cells contained a nucleic acid encoding anti-human mesothelin CAR and contained neither an exogenous nucleic acid encoding IL-7 nor an exogenous nucleic acid encoding CCL19. At the same time therewith, “CAR, IL-7, and CCL19 non-expressing T cells” (non-transfected cells: Non-infection) were produced as a CAR-negative cell control by activating peripheral blood mononuclear cells obtained from the same healthy donor by the same approach as above, but not infecting the cells with the retrovirus.

Here, the retrovirus vector was used, as described above, for introducing the nucleic acid encoding anti-human mesothelin CAR, the nucleic acid encoding IL-7, and the nucleic acid encoding CCL19 to T cells. Hence, when T cells harboring these nucleic acids proliferate by culture, some of the T cells contain the retrovirus vector in their cytoplasms. However, in most of these T cells, the nucleic acid encoding anti-human mesothelin CAR, the nucleic acid encoding IL-7, and the nucleic acid encoding CCL19 are integrated in the genome. When the nucleic acid encoding anti-human mesothelin CAR, the nucleic acid encoding IL-7, and the nucleic acid encoding CCL19 are integrated in the genome of these T cells, anti-human mesothelin CAR, IL-7, and CCL19 are expressed from the exogenous recombinant construct introduced therein.

### [Example 8] Measurement of CAR Expression by Flow Cytometry

### (Flow Cytometry Analysis)

The expression level of CAR recognizing mesothelin as an antigen was analyzed by flow cytometry analysis. The produced anti-human mesothelin CAR-IL-7/CCL19-expressing T cells were stained through reaction with recombinant human mesothelin (C-terminally containing 6-His (SEQ ID NO: 48)) (manufactured by BioLegend, Inc.), phycoerythrin (PE)-labeled anti-6-His monoclonal antibody (“anti-6-His” disclosed as SEQ ID NO: 48) (manufactured by Abcam plc), and allophycocyanin (APC)-labeled anti-CD8 monoclonal antibody (manufactured by Affymetrix/Thermo Fisher Scientific Inc.). The flow cytometer used was EC800 (manufactured by Sony Corp.). The data was analyzed using FlowJo software (manufactured by Tree Star, Inc.).

### (Results)

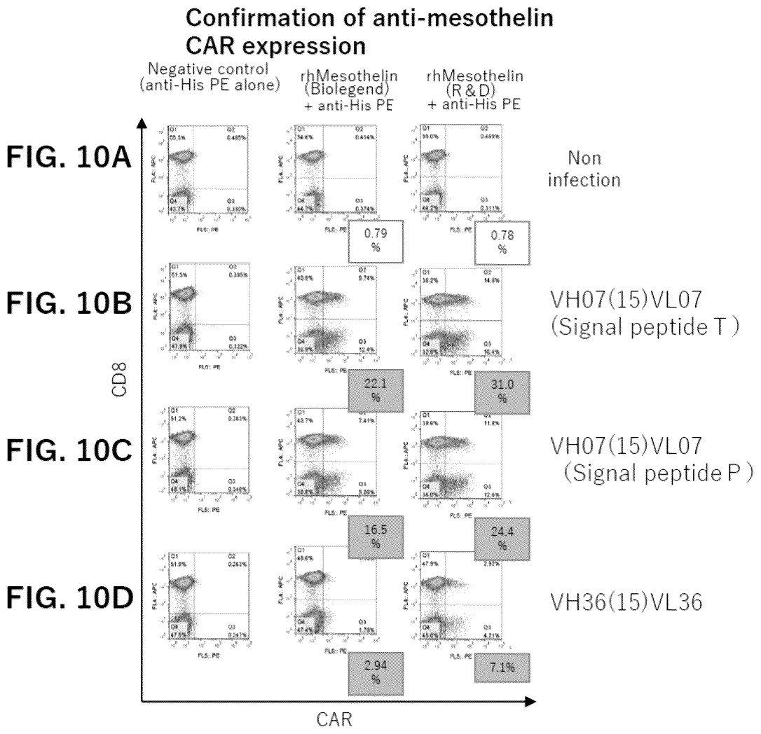

First, results of flow cytometry analysis on the anti-human mesothelin CAR-IL-7/CCL19-expressing T cells having VH07(15)VL07 (expressed through signal peptide T), VH07(15)VL07 (expressed through signal peptide P) or VH36(15)VL36 as the scFv region are shown in FIGS. 10A-10D. In FIGS. 10A-10D, the horizontal axis of each graph depicts the expression of CAR, and the longitudinal axis depicts the expression of CD8. As shown in FIGS. 10A-10D, all the three types of anti-human mesothelin CAR-IL-7/CCL19-expressing T cells were confirmed to highly express CAR as compared with the CAR, IL-7, and CCL19 non-expressing T cells (Non-infection).

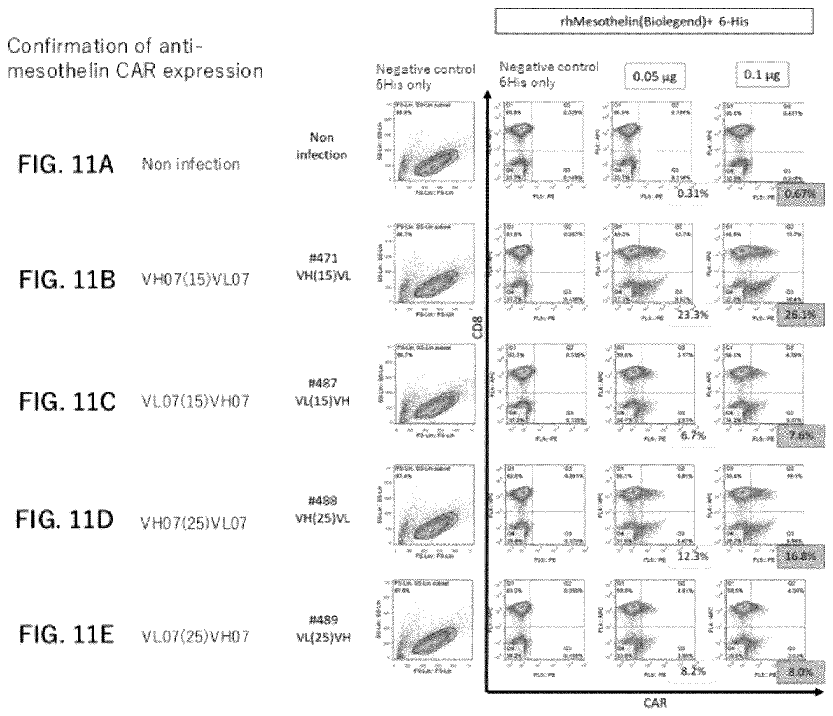

Next, results of flow cytometry analysis on the anti-human mesothelin CAR-IL-7/CCL19-expressing T cells having VH07(15)VL07, VL07(15)VH07, VH07(25)VL07, or VL07(25)VH07 as the scFv region are shown in FIGS. 11A-3E. In FIGS. 11A-3E, the abscissa of each graph depicts the expression of CAR, and the ordinate depicts the expression of CD8. FIG. 11A shows results about the CAR, IL-7, and CCL19 non-expressing T cells (Non-infection), and FIGS. 11B to 3E show results about the anti-human mesothelin CAR-IL-7/CCL19-expressing T cells having each scFv region. The numerical values in the drawing represent the percentage of each population. As shown in FIGS. 11B to 3E, the expression of CAR was confirmed in the anti-human mesothelin CAR-IL-7/CCL19-expressing T cells.

### [Example 9] Mesothelin Expression of Each Tumor Cell

### (Flow Cytometry Analysis)

The expression level of mesothelin in each tumor cell line was confirmed in order to confirm a tumor cell line expressing mesothelin. Malignant mesothelioma cell lines ACC-MESO-1, Y-MESO8A, NCI-H2052, NCI-H226, and MSTO211H, and a kidney cancer cell line A498 were stained with a commercially available anti-mesothelin antibody (Catalog Number FAB32652P: manufactured by R&D Systems, Inc.) labeled with PE. The expression of mesothelin in each tumor cell was measured by flow cytometry analysis. The staining with the PE-labeled anti-human mesothelin antibody was performed in 3 μg/sample. The flow cytometer used was EC800 (manufactured by Sony Corp.). The data was analyzed using FlowJo software (manufactured by Tree Star, Inc.).

### (Results)

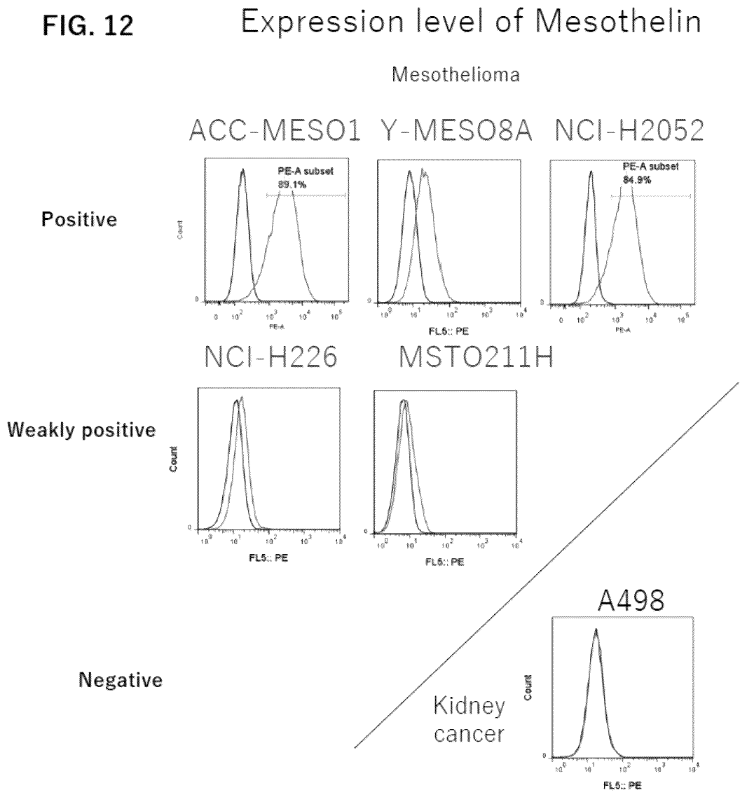

The results are shown in FIG. 12. The expression of mesothelin was confirmed in the malignant mesothelioma cell lines ACC-MESO-1, Y-MESO8A, NCI-H2052, NCI-H226, and MSTO211H. On the other hand, the expression of mesothelin was not confirmed in the kidney cancer cell line A498.

### [Example 10] Cytotoxicity Test—1

### (Co-Culture Test)

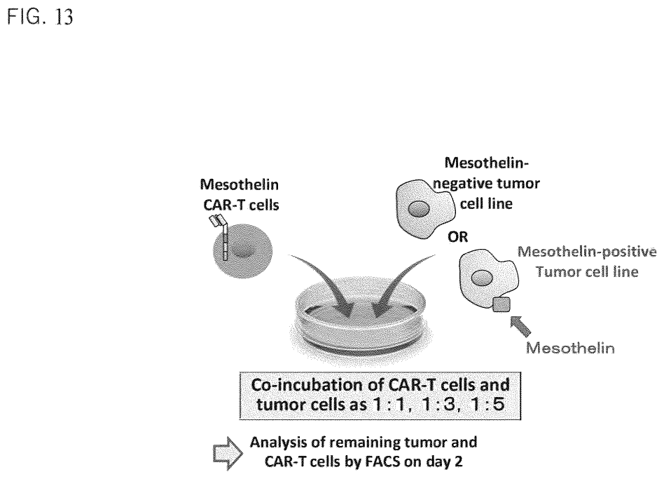

The anti-human mesothelin CAR-IL-7/CCL19-expressing T cells (scFv region: VH07(15)VL07 or VH07(25)VL07) used as an effector were adjusted to an effector:tumor cell ratio of 1:1, 1:3, or 1:5 (1:5: only for measurement and analysis of IFN-γ) with a mesothelin-positive tumor cell line (ACC-MESO-1 or NCI-H2052) or a mesothelin-negative tumor cell line (A498) on a culture plate and then co-cultured at 37° C. in an incubator. This co-culture is illustrated in FIG. 13. The culture solution used was RPMI containing 10% fetal calf serum (FCS), 1% penicillin-streptomycin (manufactured by Wako Pure Chemical Industries, Ltd.), 50 μM 2-ME (manufactured by Gibco/Thermo Fisher Scientific Inc.) and 25 mM HEPES (manufactured by Sigma-Aldrich Co. LLC). A tumor cell line surviving 2 days after the start of co-culture was measured by flow cytometry while IFN-γ produced into the culture supernatant was measured using a commercially available IFN-γ ELISA kit (manufactured by BioLegend, Inc.). The results of measuring a tumor cell line surviving 2 days after the start of co-culture by flow cytometry are shown in FIGS. 6A to 6C, and the results of measuring produced IFN-γ after the co-culture are shown in FIGS. 7A to 7C. The CAR, IL-7, and CCL19 non-expressing T cells (Non infection) were used as a control for the anti-human mesothelin CAR-IL-7/CCL19-expressing T cells. For the flow cytometry, dead cells were distinguished from live cells by staining with Zombie Yellow (manufactured by BioLegend, Inc.), and T cells were stained with PE-labeled anti-CD45 monoclonal antibody (manufactured by BioLegend, Inc.). The flow cytometer used was BD LSRFortessa X-20 (manufactured by BD Biosciences). The data was analyzed using FlowJo software (manufactured by Tree Star, Inc.).

### (Results)

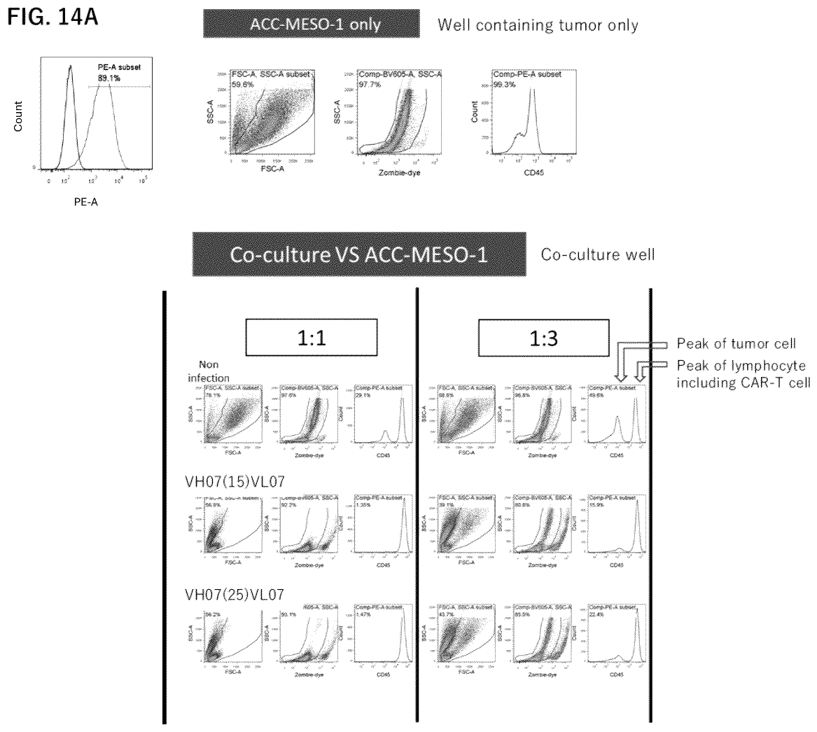

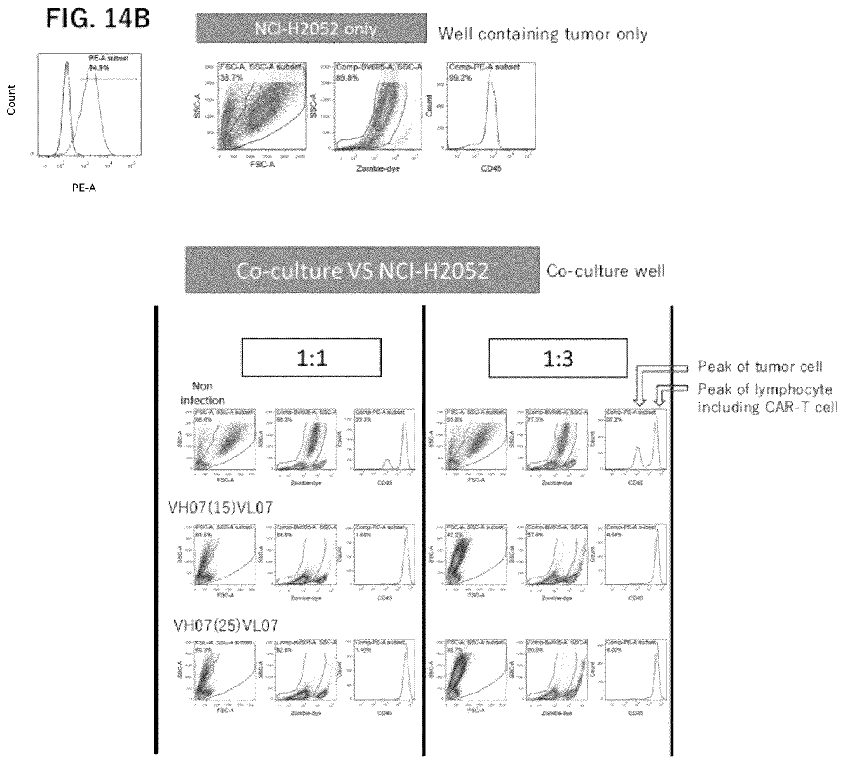

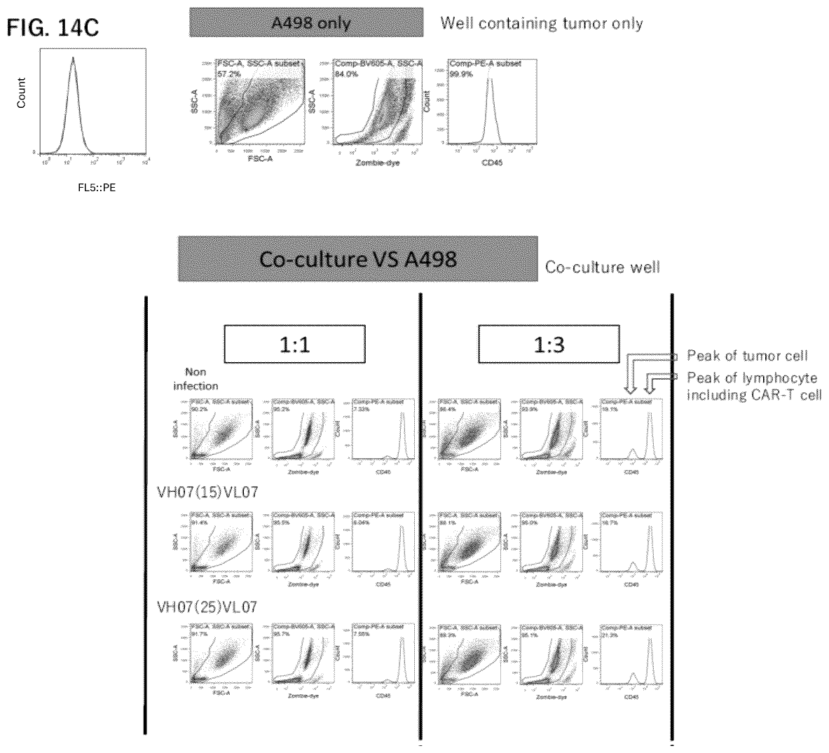

As shown in FIGS. 6A to 6C, all the target tumor cells were shown to proliferate at the same level as in wells containing tumor only, by the co-culture of the control CAR, IL-7, and CCL19 non-expressing T cells (Non-infection) with each target tumor cell. On the other hand, for the anti-human mesothelin CAR-IL-7/CCL19-expressing T cells, the target tumor cells proliferated, as with wells containing tumor only, by the co-culture with the mesothelin-negative target cells (A498: FIG. 14C), whereas an evidently decreased number of tumor cells was observed in the co-culture with the mesothelin-positive tumor cells (ACC-MESO-1: FIG. 14A, NCI-H2052: FIG. 14B) as compared with wells containing the mesothelin-positive tumor cells only and wells of co-culture with the control cells. Thus, the anti-human mesothelin CAR-IL-7/CCL19-expressing T cells were able to be confirmed to damage tumor cells in an antigen-specific manner.

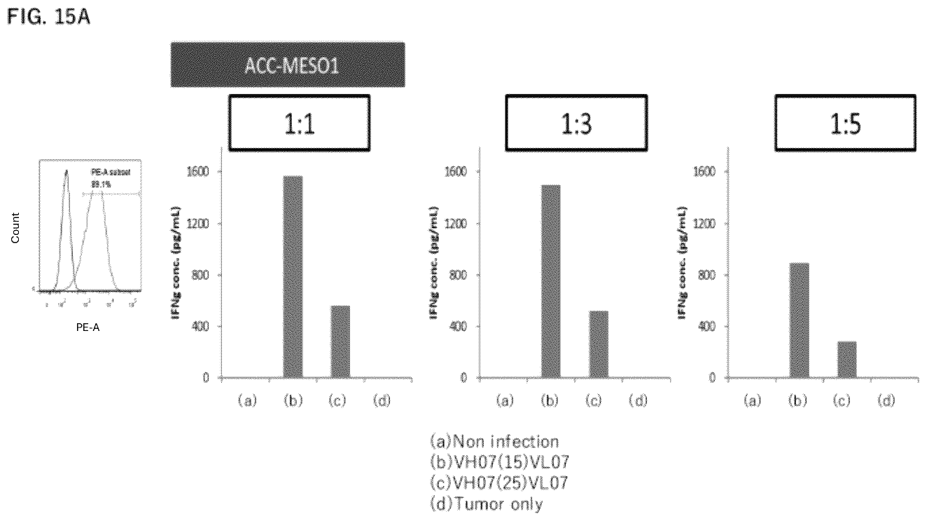

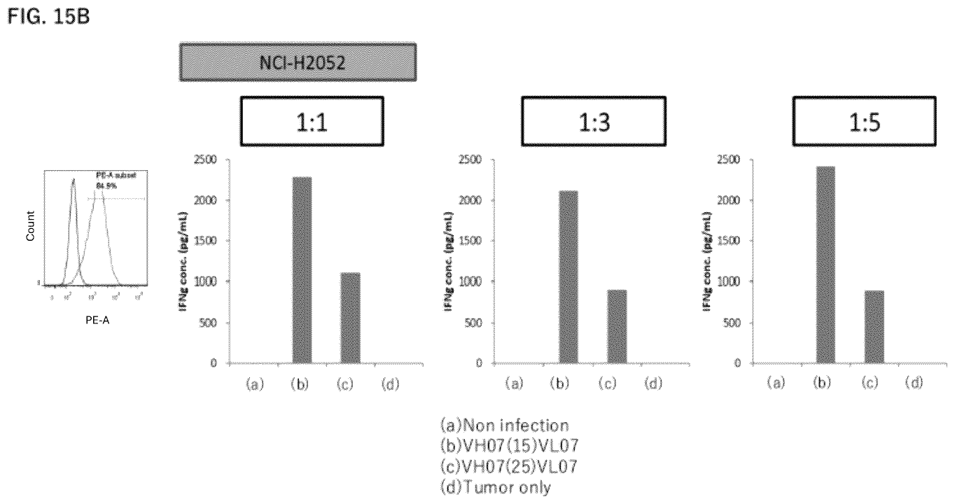

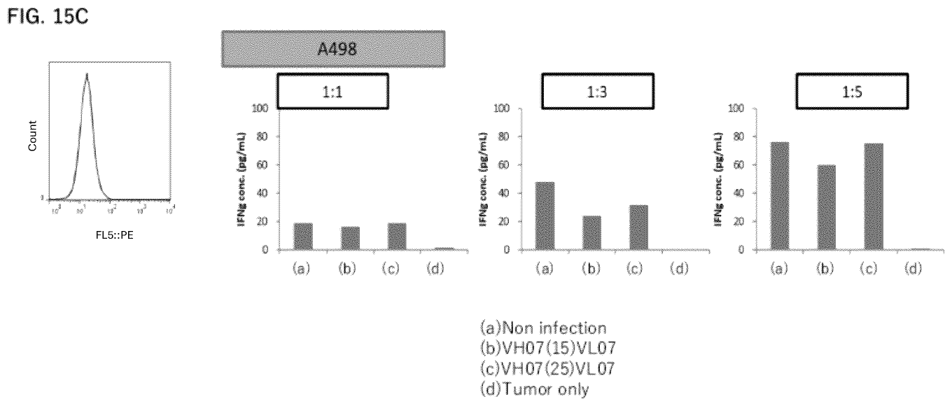

As shown in FIGS. 7A to 7C, the ELISA analysis of IFN-γ using the supernatant after the co-culture also confirmed marked production of IFN-γ only in the supernatant of the co-culture of the anti-human mesothelin CAR-IL-7/CCL19-expressing T cells with the mesothelin-positive tumor cells (ACC-MESO-1: FIG. 15A, NCI-H2052: FIG. 15B).

### [Example 11] Cytotoxicity Test—2

### (Co-Culture Test)

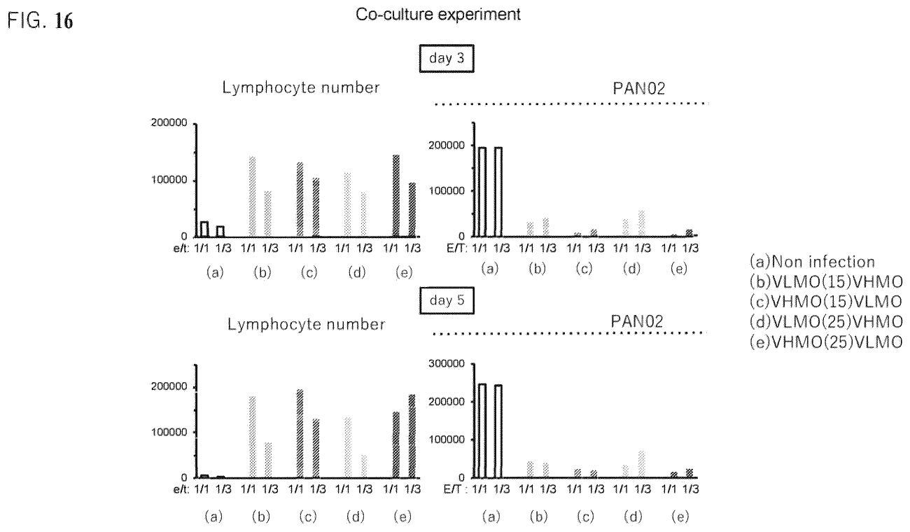

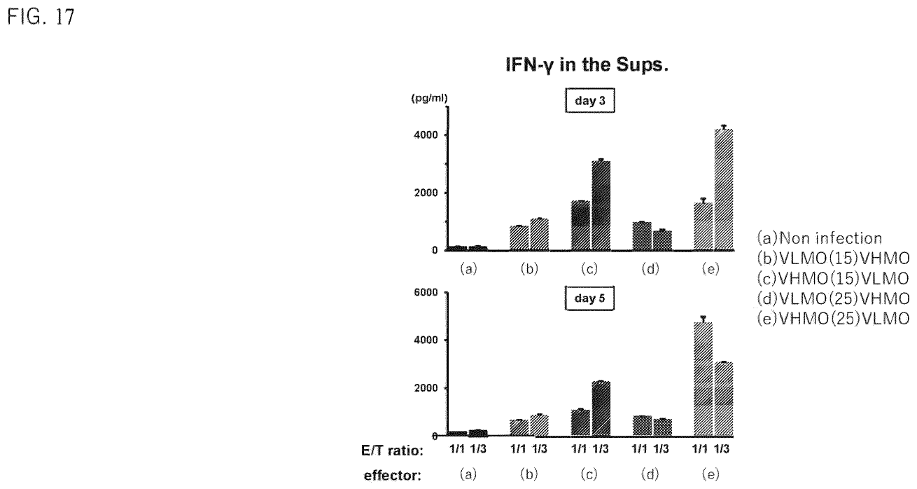

In the same way as the method of Example 10, the anti-human mesothelin CAR-IL-7/CCL19-expressing T cells (scFv region: VHMO(15)VLMO, VLMO(15)VHMO, VHMO(25)VLMO, or VLMO(25)VHMO) or the “CAR, IL-7, and CCL19 non-expressing T cells” (non-transfected cells: Non-infection) were adjusted to an effector:tumor cell ratio of 1:1 or 1:3 with a mesothelin-positive tumor cell line PAN02 or a mesothelin-negative tumor cell line on a culture plate and then co-cultured at 37° C. in an incubator. Results of measuring leukocytes or a PAN02 tumor cell line surviving 3 days or 5 days after the start of co-culture by flow cytometry are shown in FIG. 16, and results of measuring produced IFN-γ after the co-culture are shown in FIG. 17. The T cells expressing anti-human mesothelin CAR, IL-7 and CCL19, used in this Example and “Therapeutic effect in tumor model” of Example 11 mentioned later were produced in accordance with the method of Example 7 (hereinafter, referred to as “anti-human mesothelin CAR-mouse IL-7/mouse CCL19-expressing mouse T cells”) using, as the pMSGV1 retrovirus expression vector, a pMSGV1 retrovirus expression vector prepared so as to insert mouse IL-7-F2A-mouse CCL19-F2A-HSV-TK instead of the human IL-7-F2A-human CCL19-F2A-HSV-TK and mouse CD8 transmembrane region and mouse CD28-4-1BB-CD3ζ intracellular signaling region instead of the human CD8 transmembrane region and the human CD28-4-1BB-CD3ζ intracellular signaling region, and using spleen- and lymphocyte-derived mouse T cells as the T cells. The “CAR, IL-7, and CCL19 non-expressing T cells” (non-transfected cells: Non-infection) used in this Example were obtained using spleen- and lymphocyte-derived mouse T cells as the T cells.

### (Results)

As shown in FIG. 16, the anti-human mesothelin CAR-mouse IL-7/mouse CCL19-expressing mouse T cells were able to be confirmed to damage tumor cells. As shown in FIG. 17, the ELISA of IFN-γ using the supernatant after the co-culture also confirmed marked production of IFN-γ only in the supernatant of the co-culture of the anti-human mesothelin CAR-mouse IL-7/mouse CCL19-expressing mouse T cells with the PAN02 tumor cell line.

### [Example 12] Therapeutic Effect in Tumor Model

### (Administration of Anti-Mouse Mesothelin CAR-IL-7/CCL19-Expressing T Cell to Tumor Model Mouse)

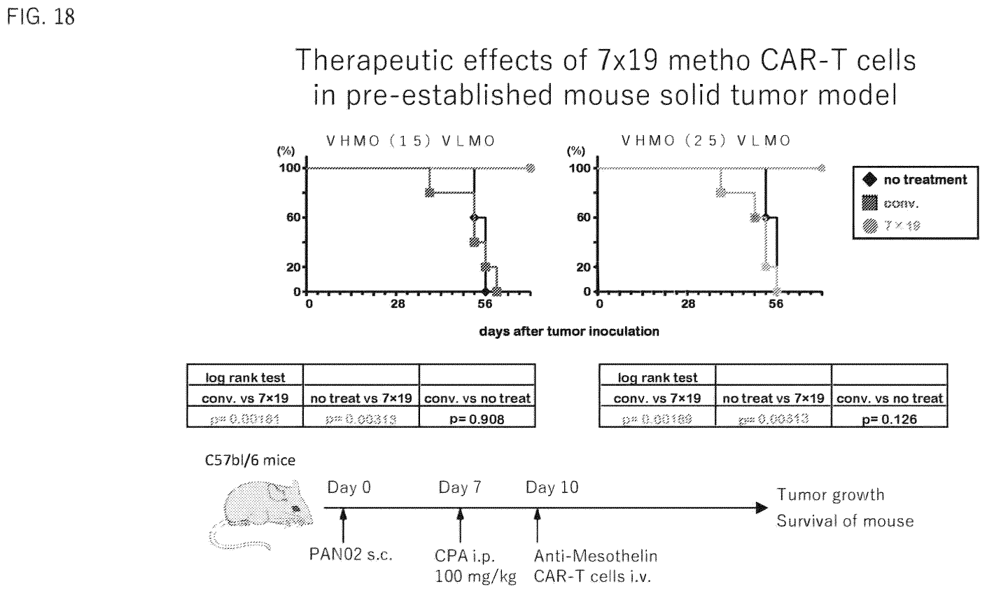

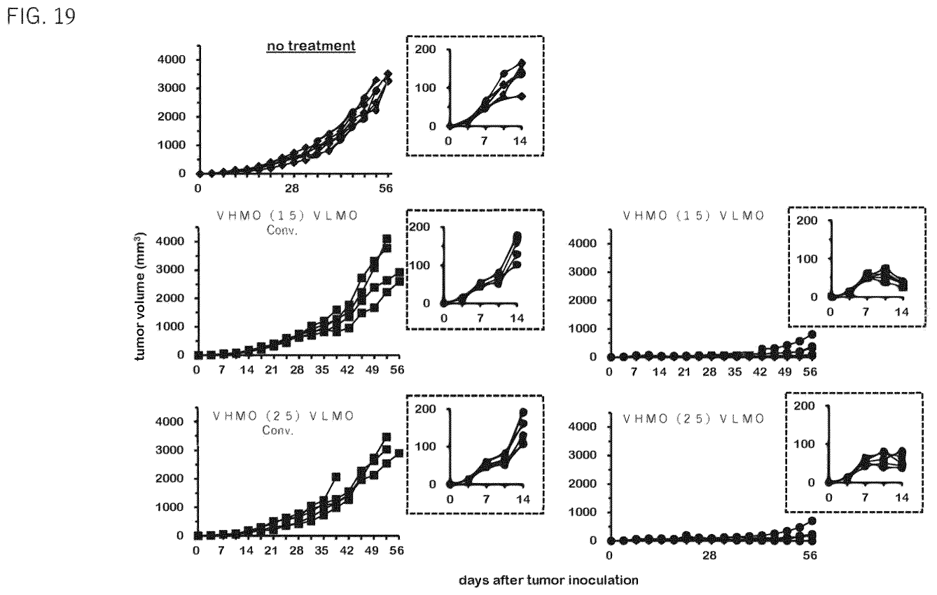

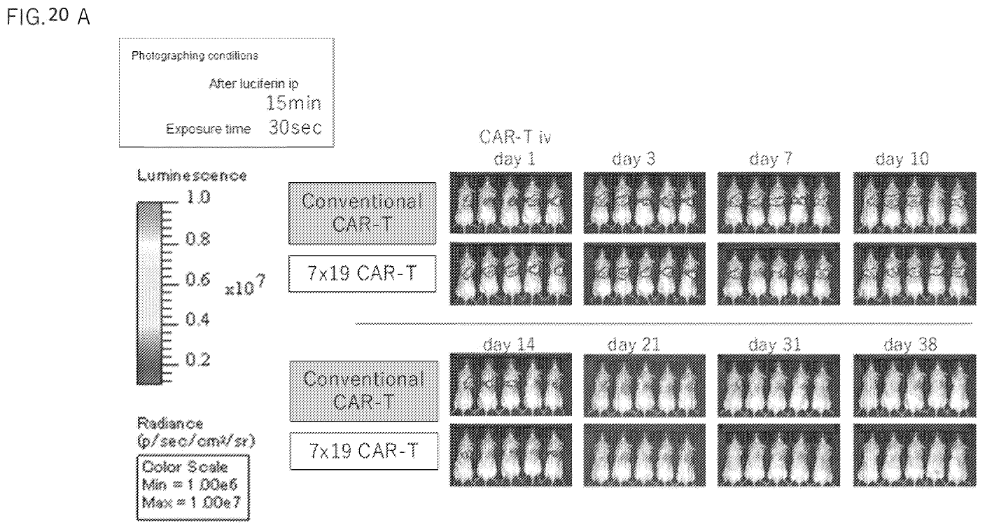

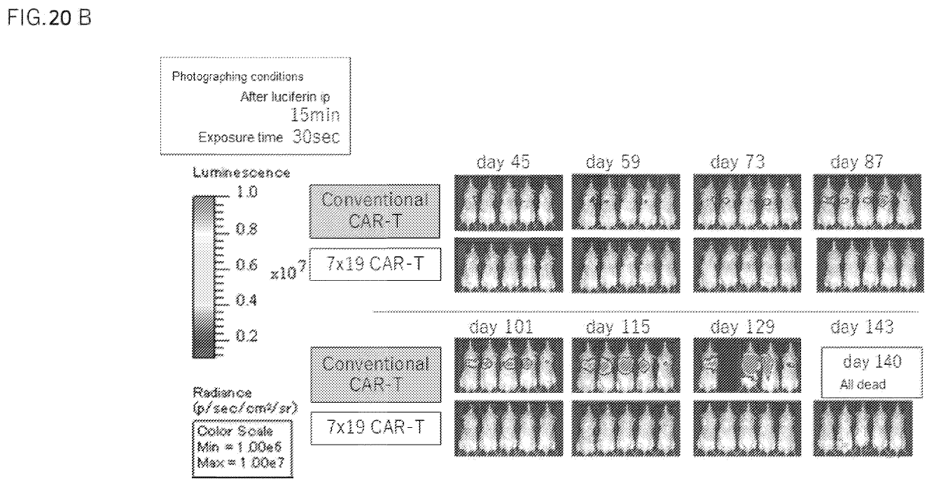

Seven- to ten-week-old C57BL/6 mice (purchased from Japan SLC, Inc.) were each subcutaneously inoculated with 5×10⁵ cells of a PAN02 pancreatic cancer cell line. On day 7 after the inoculation, an anticancer agent cyclophosphamide (CPA, 100 mg/kg) was intraperitoneally administered to the mice. On day 10, 1×10⁶ anti-human mesothelin CAR-mouse IL-7/mouse CCL19-expressing mouse T cells (scFv region: VHMO(15)VLMO, VLMO(15)VHMO, VHMO(25)VLMO, or VLMO(25)VHMO) or the “anti-human mesothelin CAR-expressing mouse T cells” produced in Example 11 were intravenously administered thereto. Results of mouse survival rates from VHMO(15)VLMO or VHMO(25)VLMO are shown in FIG. 18, and results of tumor volumes from VHMO(15)VLMO, VLMO(15)VHMO, VHMO(25)VLMO, or VLMO(25)VHMO) are shown in FIG. 19. In FIG. 18, the abscissa depicts the number of days after the subcutaneous inoculation of PAN02 (the day on which PAN02 was subcutaneously inoculated to the mice was defined as day 0), and the ordinate depicts the survival rate. In FIG. 19, the abscissa depicts the number of days after the subcutaneous inoculation of PAN02, and the ordinate depicts the tumor volume (major axis of the tumor×(minor axis of the tumor)²/2 (mm³)). “no treatment” represents a group given CPA only, “Conv.” represents a group given CPA and then the anti-human mesothelin CAR-expressing mouse T cells, and “7×19” represents a group given CPA and then the anti-human mesothelin CAR-mouse IL-7/mouse CCL19-expressing mouse T cells. The “anti-human mesothelin CAR-expressing mouse T cells” were produced in the same way as the method of Example 7 except that in the method for producing “anti-human mesothelin CAR-expressing T cells” described in Example 7, a pMSGV1 retrovirus expression vector prepared so as to insert HSV-TK instead of the human IL-7-F2A-human CCL19-F2A-HSV-TK and mouse CD8 transmembrane region and mouse CD28-4-1BB-CD3ζ intracellular signaling region instead of the human CD8 transmembrane region and the human CD28-4-1BB-CD3ζ intracellular signaling region was used as the pMSGV1 retrovirus expression vector, and spleen- and lymphocyte-derived mouse T cells were used as the T cells.

### (Results)

As shown in FIG. 18, the administration of the anti-human mesothelin CAR-IL-7/CCL19-expressing T cells according to certain aspect(s) of the present invention was found to significantly elevate survival rates. As shown in FIG. 19, the administration of the anti-human mesothelin CAR-IL-7/CCL19-expressing T cells according to certain aspect(s) of the present invention was found to evidently suppress tumor growth. This demonstrated that the anti-human mesothelin CAR-IL-7/CCL19-expressing T cells exhibit excellent antitumor activity in the tumor model mice.

### [Example 13] Therapeutic Effect in Tumor Model—2

The anti-human mesothelin CAR-IL-7/CCL19-expressing T cells were found to exhibit excellent antitumor activity. In order to confirm longer-term antitumor effects and antitumor effects on cancer other than pancreatic cancer, a human malignant pleural mesothelioma cell line was administered to immunocompromised mice to form tumor. Then, the presence or absence of tumor recurrence for 143 days was examined with or without the administration of the anti-human mesothelin CAR-IL-7/CCL19-expressing T cells. The “method for producing an ACC-MESO-1-GFP-Luc line” and the “method for activating T cells” for use in this Example are as described below.

### (Production of ACC-MESO-1-GFP-Luc Line)

A gene of green fluorescent protein-luciferase (GFP-Luc) was introduced using lentivirus to a human malignant mesothelioma cell line ACC-MESO-1, which is a mesothelin-positive tumor cell line kindly provided by Dr. Yoshitaka Sekido from Aichi Cancer Center Research Institute.

On day 0, ACC-MESO-1 was seeded at 1×10³ cells/well to a 96-well plate. The medium used was RPMI1640 (manufactured by Gibco/Thermo Fisher Scientific Inc.) supplemented with 10% FBS. On day 1, transduction was started by the addition of RediFect Red-FLuc-GFP (manufactured by PerkinElmer, Inc.), lentivirus particles for light emitting cell production, at MOI 100. In this respect, hexadimethrine bromide (manufactured by Sigma-Aldrich Co., LLC) was added at 4 μg/mL (final concentration) to the medium in order to enhance transfection efficiency. 24 hours after the virus addition (on day 2), the medium containing the virus was removed, followed by medium replacement. The culture was continued, and only cells expressing GFP were then sorted using SH800 (manufactured by Sony Corp.) to obtain ACC-MESO-1 expressing GFP, i.e., “ACC-MESO-1-GFP-Luc”.

### (Production of Anti-Human Mesothelin CAR-IL-7-CCL19-Expressing T Cell and Anti-Human Mesothelin CAR-Expressing T Cell)

In this Example 13, the IL-7/CCL19 expression-anti-human mesothelin CAR vector (having the scFv region replaced with a VH07(15)VL07 DNA fragment, and signal peptide T shown in SEQ ID NO: 25 as the signal peptide) or the Conv. anti-human mesothelin CAR vector (having the scFv region replaced with a VH07(15)VL07 DNA fragment, and signal peptide T shown in SEQ ID NO: 25 as the signal peptide) obtained in Example 7 were used.

### (Activation of T Cell)

On day 0, the culture of 2×10⁶ peripheral blood mononuclear cells collected from a healthy donor with IL-2 (manufactured by PeproTech, Inc.) was started at 37° C. in a 5% CO₂ incubator on a 6-well plate for cell culture on which 25 μL/mL RetroNectin (manufactured by Takara Bio Inc.) and 5 μg/mL anti-human CD3 monoclonal antibody (manufactured by Invitrogen Corp.) were immobilized. The culture solution used was OpTmizer CTS (manufactured by Gibco/Thermo Fisher Scientific Inc.) supplemented with 2 mM L-glutamine (manufactured by Gibco/Thermo Fisher Scientific Inc.), 1% penicillin-streptomycin (manufactured by Wako Pure Chemical Industries, Ltd.) and 2.5 μg/mL Fungizone (manufactured by Bristol-Myers Squibb Company). The cells were cultured for 3 days. On day 3, morphological change in T cells caused by activation was confirmed under a microscope to obtain activated T cells.

### (Observation of Tumor Recurrence)

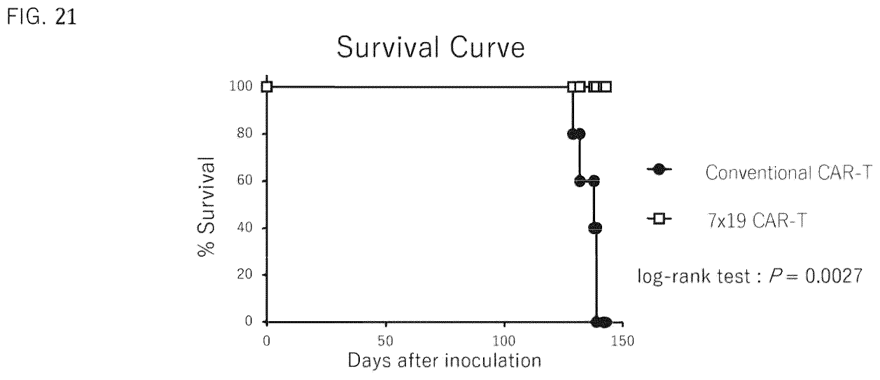

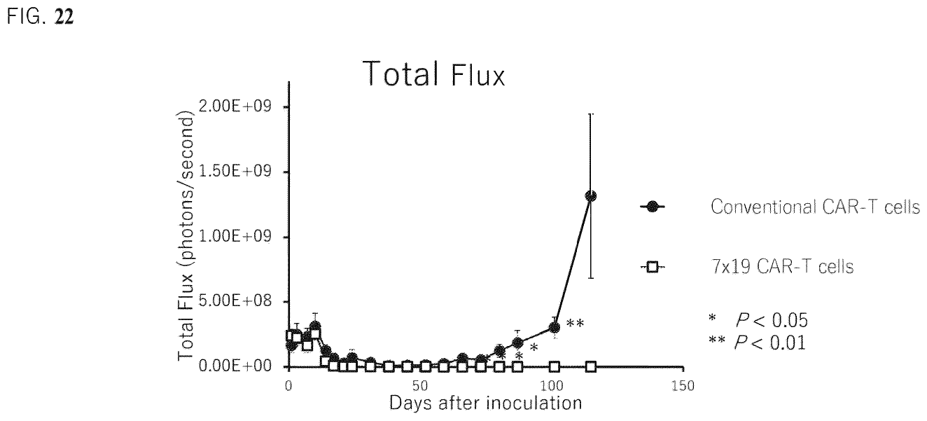

On day 0, first, the ACC-MESO-1-GFP-Luc was intrapleurally administered at 2×10⁶ cells/mouse to 8-week-old female NSG immunocompromised mice. On day 1, tumor engraftment in the pleural space was confirmed using an in vivo imaging system (IVIS). On day 1, the anti-human mesothelin CAR-expressing T cells and the anti-human mesothelin CAR-IL-7-CCL19-expressing T cells (scFv region: VH07(15)VL07) produced by the method of Example 7 and then frozen, and the T cells activated by the method described above were thawed. The anti-human mesothelin CAR-expressing T cells and the anti-human mesothelin CAR-IL-7-CCL19-expressing T cells had a CAR expression rate of 49.6% and 32.5%, respectively. Therefore, the activated T cells were added to the anti-human mesothelin CAR-expressing T cells to match their CAR expression rates. Then, a group given 1×10⁵ cells of the anti-human mesothelin CAR-expressing T cells (N=5), and a group given 1×10⁵ cells of the anti-human mesothelin CAR-IL-7-CCL19-expressing T cells (N=5) were provided. The administration of the anti-human mesothelin CAR-expressing T cells and the anti-human mesothelin CAR-IL-7-CCL19-expressing T cells was performed by intravenous administration from the tail veins. On day 3 and subsequent days, tumor fluorescence intensity was measured (total flux (photons/sec)) using IVIS. The results are shown in FIGS. 12A and 12B. The relationship between the number of days from administration and the survival rates of the mice in the results is shown in a graph form in FIG. 21, and the relationship between the number of days from administration and the total quantity of fluorescence (photons/second) is shown in a graph form in FIG. 22. In FIGS. 12A, 12B, 13, and 14, the mice given the anti-human mesothelin CAR-IL-7-CCL19-expressing T cells are shown by “7×19 CAR-T”, and the mice given the anti-human mesothelin CAR-expressing T cells are shown by “Conventional CAR-T”. In this Example 13, the influence of endogenous T cells of recipients was excluded because NSG immunocompromised mice deficient in endogenous T cells were used as the recipients. Thus, the effects of the administered anti-human mesothelin CAR-IL-7-CCL19-expressing T cells themselves were evaluated.

As shown in FIGS. 12A, 12B, 13, and 14, tumor fluorescence intensity was rarely observed in both 7×19 CAR-T and Conventional CAR-T on day 21. Tumor fluorescence was not observed in 7×19 CAR-T up to day 143, confirming that recurrence was completely suppressed. On the other hand, tumor fluorescence was observed in Conventional CAR-T from about day 45, and tumor fluorescence intensity elevated on day 115 with one mouse dead on day 129 and the remaining four mice dead on day 143. This demonstrated that the administered CAR-IL-7-CCL19-expressing T cells have cytotoxic activity not only against pancreatic cancer but against cancer (e.g., human malignant pleural mesothelioma) cells expressing human mesothelin, and have long-term antitumor effects.

Aspects of the invention described herein may also be described as follows:
1. An isolated immunocompetent cell expressing at least one cell surface molecule specifically recognizing a cancer antigen, interleukin 7 (IL-7), and at least one of CCL19 and CCL21.
2. The immunocompetent cell according to 1, wherein the immunocompetent cell comprises a nucleic acid encoding IL-7 introduced from outside the cell and at least one of the nucleic acids encoding CCL19 and CCL21 introduced from outside the cell.
3. The immunocompetent cell according to 1, wherein the cell surface molecule that specifically recognizes the cancer antigen comprises a T cell receptor or a chimeric antigen receptor that specifically recognizes the cancer antigen.
4. The immunocompetent cell according to 1, wherein the immunocompetent cell is selected from the group consisting of a natural killer cell (NK cell), a B cell, an antigen-presenting cell and a granulocyte.
5. The immunocompetent cell according to 1, wherein the cancer antigen comprises WT1, MART-1, NY-ESO-1, MAGE-A1, MAGE-A3, MAGE-A4, Glypican-3, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GD2, or GM2.
6. A composition comprising one or more expression vectors for generating the isolated immunocompetent cell according to claim 1, the composition comprising any of the following (a) to (e):
   a. an expression vector containing a nucleic acid encoding a cell surface molecule specifically recognizing a cancer antigen, a nucleic acid encoding IL-7, and a nucleic acid encoding at least one of CCL19 and CCL21;
   b. the following two expression vectors (b-1) and (b-2):
   c. (b-1) an expression vector containing a nucleic acid encoding a cell surface molecule specifically recognizing a cancer antigen; and
   d. (b-2) an expression vector containing a nucleic acid encoding IL-7 and a nucleic acid encoding at least one of CCL19 and CCL21;
   e. the following two expression vectors (c-1) and (c-2):
   f. (c-1) an expression vector containing a nucleic acid encoding a cell surface molecule specifically recognizing a cancer antigen, and a nucleic acid encoding IL-7; and
   g. (c-2) an expression vector containing a nucleic acid encoding at least one of CCL19 and CCL21;
   h. the following two expression vectors (d-1) and (d-2):
   i. (d-1) an expression vector containing a nucleic acid encoding IL-7; and
   j. (d-2) an expression vector containing a nucleic acid encoding a cell surface molecule specifically recognizing a cancer antigen, and a nucleic acid encoding at least one of CCL19 and CCL21; and
   k. the following three expression vectors (e-1), (e-2) and (e-3):
   l. (e-1) an expression vector containing a nucleic acid encoding a cell surface molecule specifically recognizing a cancer antigen;
   m. (e-2) an expression vector containing a nucleic acid encoding IL-7; and
   n. (e-3) an expression vector containing a nucleic acid encoding at least one of CCL19 and CCL21.
7. The composition according to 6, wherein the cell surface molecule specifically recognizing a cancer antigen comprises a T cell receptor or a chimeric antigen receptor specifically recognizing the cancer antigen.
8. The composition according to 6, wherein
   the nucleic acid encoding a cell surface molecule specifically recognizing a cancer antigen, the nucleic acid encoding IL-7, and the nucleic acid encoding at least one of CCL19 and CCL21 in the expression vector (a); or
   the nucleic acid encoding IL-7 and the nucleic acid encoding at least one of CCL19 and CCL21 in the expression vector (b-2); or
   the nucleic acid encoding a cell surface molecule specifically recognizing a cancer antigen, and the nucleic acid encoding IL-7 in the expression vector (c-1); or
   the nucleic acid encoding a cell surface molecule specifically recognizing a cancer antigen, and the nucleic acid encoding at least one of CCL19 and CCL21 in the expression vector (d-2)
   are linked via a sequence encoding a self-cleaving peptide.
9. The composition according to 6, wherein the expression vector contains a nucleic acid encoding a suicide gene.
10. An anticancer agent comprising the isolated immunocompetent cell according to 1 and a pharmaceutically acceptable additive.
11. The anticancer agent according to 10, wherein the anticancer agent is administered in combination with cyclophosphamide or fludarabine.
12. A method of treating a tumor in a subject, comprising administering to the subject in need thereof an effective amount of an immunocompetent cell derived from a mammal or separated from a mammal, wherein the immunocompetent cell expresses a chimeric antigen receptor (CAR) specifically recognizing human mesothelin, interleukin 7 (IL-7), and chemokine (C-C motif) ligand 19 (CCL19),
   the CAR comprising a single chain antibody, a transmembrane region, and a signaling region that induces activation of the immunocompetent cell,
   wherein the single chain antibody in the CAR is (1-1) a single chain antibody comprising a heavy chain variable region comprising heavy chain CDR1 consisting of the amino acid sequence shown by SEQ ID NO: 13, heavy chain CDR2 consisting of the amino acid sequence shown by SEQ ID NO: 14, and heavy chain CDR3 consisting of the amino acid sequence shown by SEQ ID NO: 15, and a light chain variable region comprising light chain CDR1 consisting of the amino acid sequence shown by SEQ ID NO: 16, light chain CDR2 consisting of the amino acid sequence shown by SEQ ID NO: 17, and light chain CDR3 consisting of the amino acid sequence shown by SEQ ID NO: 18.
13. The method of 12, wherein the subject is a human.
14. The method of 12, wherein the immunocompetent cell comprises an exogenous nucleic acid encoding a CAR specifically recognizing human mesothelin, an exogenous nucleic acid encoding IL-7, and an exogenous nucleic acid encoding CCL19.
15. The method of 14, wherein the exogenous nucleic acid encoding IL-7, and the exogenous nucleic acid encoding CCL19 are an exogenous nucleic acid encoding human IL-7, and an exogenous nucleic acid encoding human CCL19.
16. The method of 14, wherein the exogenous nucleic acid encoding a CAR specifically recognizing human mesothelin, the exogenous nucleic acid encoding IL-7, and the exogenous nucleic acid encoding CCL19 are integrated in a genome.
17. The method of ¥12, wherein the heavy chain variable region other than the CDRs consists of an amino acid sequence having 85% or higher sequence identity to the amino acid sequence shown by SEQ ID NO: 1 other than the CDRs, and the light chain variable region other than the CDRs consists of an amino acid sequence having 85% or higher sequence identity to the amino acid sequence shown by SEQ ID NO: 2 other than the CDRs thereof.
18. The method of 12, wherein the single chain antibody in the CAR is
   (1-3) a single chain antibody comprising a heavy chain variable region consisting of the amino acid sequence shown by SEQ ID NO: 1, and a light chain variable region consisting of the amino acid sequence shown by SEQ ID NO: 2.
19. The method of 12, wherein the transmembrane region in the CAR comprises an amino acid sequence having 85% or higher sequence identity to the amino acid sequence shown by SEQ ID NO: 7.
20. The method of 12, wherein the signaling region that induces the activation of the immunocompetent cell in the CAR comprises the amino acid sequences shown by SEQ ID NOs: 9 and 10.
21. The method of 12, wherein the heavy chain variable region and the light chain variable region are connected via a peptide linker consisting of a 2- to 30-amino acid sequence.
22. The method of claim 21, wherein the peptide linker consists of the amino acid sequence shown by SEQ ID NO: 26 or SEQ ID NO: 27.
23. The method of 12, wherein the single chain antibody in the CAR is
   (1-4) a single chain antibody sequentially comprising a heavy chain variable region consisting of the amino acid sequence shown by SEQ ID NO: 1, a peptide linker consisting of the amino acid sequence shown by SEQ ID NO: 26, and a light chain variable region consisting of the amino acid sequence shown by SEQ ID NO: 2.
24. The method of 12, wherein the immunocompetent cell is a T cell.
25. The method of 12, wherein the mammal is a human.
26. The method of 12, wherein the light chain variable region is positioned on the C-terminal side of the heavy chain variable region.
27. The method of claim 12, wherein in the CAR, in order from the N terminus is the single chain antibody, the transmembrane region, and the signaling region that induces the activation of the immunocompetent cell.
28. A method of reducing malignant tumor recurrence in a subject, comprising administering to the subject in need thereof an effective amount of an immunocompetent cell derived from a mammal or separated from a mammal, wherein the immunocompetent cell expresses a chimeric antigen receptor (CAR) specifically recognizing human mesothelin, interleukin 7 (IL-7), and chemokine (C-C motif) ligand 19 (CCL19),
   the CAR comprising a single chain antibody, a transmembrane region, and a signaling region that induces activation of the immunocompetent cell,
   wherein the single chain antibody in the CAR is (1-1) a single chain antibody comprising a heavy chain variable region comprising heavy chain CDR1 consisting of the amino acid sequence shown by SEQ ID NO: 13, heavy chain CDR2 consisting of the amino acid sequence shown by SEQ ID NO: 14, and heavy chain CDR3 consisting of the amino acid sequence shown by SEQ ID NO: 15, and a light chain variable region comprising light chain CDR1 consisting of the amino acid sequence shown by SEQ ID NO: 16, light chain CDR2 consisting of the amino acid sequence shown by SEQ ID NO: 17, and light chain CDR3 consisting of the amino acid sequence shown by SEQ ID NO: 18.
29. The method of 28, wherein the subject is a human.
30. A method of treating a tumor and suppressing tumor recurrence in a subject, comprising administering to the subject in need thereof an effective amount of an immunocompetent cell derived from a mammal or separated from a mammal, wherein the immunocompetent cell expresses a chimeric antigen receptor (CAR) specifically recognizing human mesothelin, interleukin 7 (IL-7), and chemokine (C-C motif) ligand 19 (CCL19), the CAR comprising a single chain antibody, a transmembrane region, and a signaling region that induces activation of the immunocompetent cell,
   wherein the single chain antibody in the CAR is (1-1) a single chain antibody comprising a heavy chain variable region comprising heavy chain CDR1 consisting of the amino acid sequence shown by SEQ ID NO: 13, heavy chain CDR2 consisting of the amino acid sequence shown by SEQ ID NO: 14, and heavy chain CDR3 consisting of the amino acid sequence shown by SEQ ID NO: 15, and a light chain variable region comprising light chain CDR1 consisting of the amino acid sequence shown by SEQ ID NO: 16, light chain CDR2 consisting of the amino acid sequence shown by SEQ ID NO: 17, and light chain CDR3 consisting of the amino acid sequence shown by SEQ ID NO: 18.
31. The method of 30, wherein the subject is a human.
32. The method of 12, wherein the signaling region that induces the activation of the immunocompetent cell in the CAR comprises a polypeptide of a 4-1BB intracellular region and a polypeptide of a CD3ζ intracellular region, and the signaling region that induces the activation of the immunocompetent cell in the CAR does not comprise a polypeptide of a CD28 intracellular region.

The present application is based on Japanese Patent Applications Nos. 2017-247109 (filed on Dec. 24, 2017) and JP 2016-053913 (filed Mar. 17, 2016), the contents of which are incorporated herein in their entirety.

1. An isolated immunocompetent cell expressing at least one cell surface molecule specifically recognizing a cancer antigen, interleukin 7 (IL-7), and at least one of CCL19 and CCL21.

2. The immunocompetent cell according to claim 1, wherein the immunocompetent cell comprises a nucleic acid encoding IL-7 introduced from outside the cell and at least one of the nucleic acids encoding CCL19 and CCL21 introduced from outside the cell.

3. The immunocompetent cell according to claim 1, wherein the cell surface molecule that specifically recognizes the cancer antigen comprises a T cell receptor or a chimeric antigen receptor that specifically recognizes the cancer antigen.

4. The immunocompetent cell according to claim 1, wherein the immunocompetent cell is selected from the group consisting of a natural killer cell (NK cell), a B cell, an antigen-presenting cell and a granulocyte.

5. The immunocompetent cell according to claim 1, wherein the cancer antigen comprises WT1, MART-1, NY-ESO-1, MAGE-A1, MAGE-A3, MAGE-A4, Glypican-3, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GD2, or GM2.

6. A composition comprising one or more expression vectors for generating the isolated immunocompetent cell according to claim 1, the composition comprising any of the following (a) to (e):
(a) an expression vector containing a nucleic acid encoding a cell surface molecule specifically recognizing a cancer antigen, a nucleic acid encoding IL-7, and a nucleic acid encoding at least one of CCL19 and CCL21;
(b) the following two expression vectors (b-1) and (b-2):
(b-1) an expression vector containing a nucleic acid encoding a cell surface molecule specifically recognizing a cancer antigen; and
(b-2) an expression vector containing a nucleic acid encoding IL-7 and a nucleic acid encoding at least one of CCL19 and CCL21;
(c) the following two expression vectors (c-1) and (c-2):
(c-1) an expression vector containing a nucleic acid encoding a cell surface molecule specifically recognizing a cancer antigen, and a nucleic acid encoding IL-7; and
(c-2) an expression vector containing a nucleic acid encoding at least one of CCL19 and CCL21;
(d) the following two expression vectors (d-1) and (d-2):
(d-1) an expression vector containing a nucleic acid encoding IL-7; and
(d-2) an expression vector containing a nucleic acid encoding a cell surface molecule specifically recognizing a cancer antigen, and a nucleic acid encoding at least one of CCL19 and CCL21; and
(e) the following three expression vectors (e-1), (e-2) and (e-3):
(e-1) an expression vector containing a nucleic acid encoding a cell surface molecule specifically recognizing a cancer antigen;
(e-2) an expression vector containing a nucleic acid encoding IL-7; and
(e-3) an expression vector containing a nucleic acid encoding at least one of CCL19 and CCL21.

7. The composition according to claim 6, wherein the cell surface molecule specifically recognizing a cancer antigen comprises a T cell receptor or a chimeric antigen receptor specifically recognizing the cancer antigen.

8. The composition according to claim 6, wherein the nucleic acid encoding a cell surface molecule specifically recognizing a cancer antigen, the nucleic acid encoding IL-7, and the nucleic acid encoding at least one of CCL19 and CCL21 in the expression vector (a); or
the nucleic acid encoding IL-7 and the nucleic acid encoding at least one of CCL19 and CCL21 in the expression vector (b-2); or
the nucleic acid encoding a cell surface molecule specifically recognizing a cancer antigen, and the nucleic acid encoding IL-7 in the expression vector (c-1); or
the nucleic acid encoding a cell surface molecule specifically recognizing a cancer antigen, and the nucleic acid encoding at least one of CCL19 and CCL21 in the expression vector (d-2)
are linked via a sequence encoding a self-cleaving peptide.

9. The composition according to claim 6, wherein the expression vector contains a nucleic acid encoding a suicide gene.

10. An anticancer agent comprising the isolated immunocompetent cell according to claim 1 and a pharmaceutically acceptable additive.

11. The anticancer agent according to claim 10, wherein the anticancer agent is administered in combination with cyclophosphamide or fludarabine.

12. A method of treating a tumor in a subject, comprising administering to the subject in need thereof an effective amount of an immunocompetent cell derived from a mammal or separated from a mammal, wherein the immunocompetent cell expresses a chimeric antigen receptor (CAR) specifically recognizing human mesothelin, interleukin 7 (IL-7), and chemokine (C-C motif) ligand 19 (CCL19),
the CAR comprising a single chain antibody, a transmembrane region, and a signaling region that induces activation of the immunocompetent cell,
wherein the single chain antibody in the CAR is (1-1) a single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 30, light chain CDR2 consisting of the amino acid sequence shown by SEQ ID NO: 31, and light chain CDR3 consisting of the amino acid sequence shown by SEQ ID NO: 32.

13. The method of claim 12, wherein the subject is a human.

14. The method of claim 12, wherein the immunocompetent cell comprises an exogenous nucleic acid encoding a CAR specifically recognizing human mesothelin, an exogenous nucleic acid encoding IL-7, and an exogenous nucleic acid encoding CCL19.

15. The method of claim 14, wherein the exogenous nucleic acid encoding IL-7, and the exogenous nucleic acid encoding CCL19 are an exogenous nucleic acid encoding human IL-7, and an exogenous nucleic acid encoding human CCL19.

16. The method of claim 14, wherein the exogenous nucleic acid encoding a CAR specifically recognizing human mesothelin, the exogenous nucleic acid encoding IL-7, and the exogenous nucleic acid encoding CCL19 are integrated in a genome.

17. The method of claim 12, wherein the heavy chain variable region other than the CDRs consists of an amino acid sequence having 85% or higher sequence identity to the amino acid sequence shown by SEQ ID NO: 15 other than the CDRs, and the light chain variable region other than the CDRs consists of an amino acid sequence having 85% or higher sequence identity to the amino acid sequence shown by SEQ ID NO: 16 other than the CDRs thereof.

18. The method of claim 12, wherein the single chain antibody in the CAR is
(1-3) a single chain antibody comprising a heavy chain variable region consisting of the amino acid sequence shown by SEQ ID NO: 1, and a light chain variable region consisting of the amino acid sequence shown by SEQ ID NO: 2.

19. The method of claim 12, wherein the transmembrane region in the CAR comprises an amino acid sequence having 85% or higher sequence identity to the amino acid sequence shown by SEQ ID NO: 21.

20. The method of claim 12, wherein the signaling region that induces the activation of the immunocompetent cell in the CAR comprises the amino acid sequences shown by SEQ ID NOs: 9 and 10.

21. The method of claim 12, wherein the heavy chain variable region and the light chain variable region are connected via a peptide linker consisting of a 2- to 30-amino acid sequence.

22. The method of claim 21, wherein the peptide linker consists of the amino acid sequence shown by SEQ ID NO: 40 or SEQ ID NO: 41.

23. The method of claim 12, wherein the single chain antibody in the CAR is—
(1-4) a single chain antibody sequentially comprising a heavy chain variable region consisting of the amino acid sequence shown by SEQ ID NO: 1, a peptide linker consisting of the amino acid sequence shown by SEQ ID NO: 40, and a light chain variable region consisting of the amino acid sequence shown by SEQ ID NO: 2.

24. The method of claim 12, wherein the immunocompetent cell is a T cell.

25. The method of claim 12, wherein the mammal is a human.

26. The method of claim 12, wherein the light chain variable region is positioned on the C-terminal side of the heavy chain variable region.

27. The method of claim 12, wherein in the CAR, in order from the N terminus is the single chain antibody, the transmembrane region, and the signaling region that induces the activation of the immunocompetent cell.

28. A method of reducing malignant tumor recurrence in a subject, comprising administering to the subject in need thereof an effective amount of an immunocompetent cell derived from a mammal or separated from a mammal, wherein the immunocompetent cell expresses a chimeric antigen receptor (CAR) specifically recognizing human mesothelin, interleukin 7 (IL-7), and chemokine (C-C motif) ligand 19 (CCL19),
the CAR comprising a single chain antibody, a transmembrane region, and a signaling region that induces activation of the immunocompetent cell,
wherein the single chain antibody in the CAR is (1-1) a single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 30, light chain CDR2 consisting of the amino acid sequence shown by SEQ ID NO: 31, and light chain CDR3 consisting of the amino acid sequence shown by SEQ ID NO: 32.

29. The method of claim 28, wherein the subject is a human.

30. A method of treating a tumor and suppressing tumor recurrence in a subject, comprising administering to the subject in need thereof an effective amount of an immunocompetent cell derived from a mammal or separated from a mammal, wherein the immunocompetent cell expresses a chimeric antigen receptor (CAR) specifically recognizing human mesothelin, interleukin 7 (IL-7), and chemokine (C-C motif) ligand 19 (CCL19),
the CAR comprising a single chain antibody, a transmembrane region, and a signaling region that induces activation of the immunocompetent cell,
wherein the single chain antibody in the CAR is (1-1) a single chain antibody comprising a heavy chain variable region comprising heavy chain CDR1 consisting of the amino acid sequence shown by SEQ ID NO: 27, heavy chain CDR2 consisting of the amino acid sequence shown by SEQ ID NO: 28, and heavy chain CDR3 consisting of the amino acid sequence shown by SEQ ID NO: 15, and a light chain variable region comprising light chain CDR1 consisting of the amino acid sequence shown by SEQ ID NO: 30, light chain CDR2 consisting of the amino acid sequence shown by SEQ ID NO: 31, and light chain CDR3 consisting of the amino acid sequence shown by SEQ ID NO: 32.
